(19) United States
(12) Patent Application Publication
(10) Pub. No.: US 2024/0299351 A1
(43) Pub. Date: Sep. 12, 2024

(54) MELANIN MATERIALS FOR TISSUE REPAIR

(71) Applicant: Northwestern University, Evanston, IL (US)

(72) Inventors: Nathan C. GIANNESCHI, Wilmette, IL (US); Kurt Q. LU, Chicago, IL (US); Zofia Elzbieta SIWICKA, Evanston, IL (US); Naneki Chelsea COLLINS-MCCALLUM, San Diego, CA (US); Dauren BIYASHEV, Chicago, IL (US); Ummiye Venus ONAY, Skokie, IL (US); Michael Mark DEMCZUK, Chicago, IL (US)

(73) Assignee: Northwestern University, Evanston, IL (US)

(21) Appl. No.: 18/288,473

(22) PCT Filed: Apr. 28, 2022
(86) PCT No.: PCT/US22/26669

§ 371 Date: Oct. 26, 2023

Related U.S. Application Data
(60) Provisional application No. 63/181,055, filed on Apr. 28, 2021.

(51) Int. Cl. A61K 31/404; A61K 9/00; A61K 9/14; A61L 26/00; A61P 17/02

(52) U.S. Cl. CPC A61K 31/404; A61K 9/0014; A61K 9/14; A61L 26/0052; A61P 17/02

(57) ABSTRACT

Aspects disclosed herein include a method for treatment of a subject, the method comprising: topically administering a melanin formulation having an artificial melanin material (thereby administering artificial melanin material) to (or, to a site of) damaged skin of the subject; wherein the administered artificial melanin material comprises extracellular artificial melanin material at (or, at the site of) the damaged skin; and facilitating skin healing in the wound via at least the extracellular artificial melanin material; wherein the step of facilitating skin healing comprises at least a portion of the extracellular artificial melanin material performing a therapeutic extracellular activity.

### CROSS-REFERENCE TO RELATED APPLICATIONS

This application claims the benefit of priority to U.S. Provisional Patent Application No. 63/181,055, filed Apr. 28, 2021, which is hereby incorporated by reference in its entirety.

### STATEMENT REGARDING FEDERALLY SPONSORED RESEARCH OR DEVELOPMENT

This invention was made with government support under grant number AR071168 and AR079795 awarded by the National Institutes of Health and grant number FA9550-18-1-0142 awarded by the Air Force Office of Scientific Research. The government has certain rights in the invention.

### BACKGROUND OF INVENTION

Melanin is a pervasive biomaterial widely found in different organisms. In humans, melanin is produced by melanosome organelles, which are found in melanocytes. Uniquely, melanocytes are the only known cells that can excrete organelles extracellularly. Melanin is most notably known as a dark brown or black pigment in the skin and functions as a broadband radiation adsorbent. Melanosome production increases in response to increased UV exposure aiding in UV radiation protection. Properties of melanin are not limited photoprotection; it has a myriad of other functions uncharacteristic to those of biological pigments that can account for its ubiquitous presence in nature including structural coloration, metal chelation, small molecule absorption, and thermoregulation.

These diverse functions can be attributed to melanin's chemical nature and morphology. Melanin may exhibit many intermolecular interactions such as hydrogen-bonding, pi-pi stacking, and covalent interactions. The abundance of different intermolecular interactions is due to melanin's various functional groups. Due to the different oxidation states of its functional groups, melanin can accept and donate electrons allowing for redox activity. Furthermore, it has antioxidant activity because of its electron donation capabilities. Melanin can quench radical oxygen species (ROS). The ability to prevent cell damage via ROS pathways may be a pathway by which melanin protects cells against radiation to prevent or mitigate cellular damage.

Investigations of therapeutic uses for melanin, especially artificial melanin materials, beyond intracellular protective functions are limited, however.

### SUMMARY OF THE INVENTION

Included herein are methods for facilitating tissue healing, particularly skin healing, in a living subject, such as for treatment or remediation of a damaged skin, such as in the case of a closed wound, in the living subject, using a melanin formulation having a melanin material. Unexpectedly, for example, tissue healing may be facilitated by administered melanin material that is present extracellularly at the damaged skin.

Aspects disclosed herein include a method for treatment of a subject, the method comprising: topically administering a melanin formulation having an artificial melanin material (thereby administering artificial melanin material) to (or, to a site of) damaged skin of the subject; wherein the administered artificial melanin material comprises an extracellular artificial melanin material at (or, at the site of) the damaged skin; and facilitating skin healing at (or, in) the damaged skin via at least the extracellular artificial melanin material; wherein the step of facilitating skin healing comprises at least a portion of the extracellular artificial melanin material performing a therapeutic extracellular activity.

Optionally, the artificial melanin material comprises porous artificial melanin material, artificial melanin particles, and/or porous artificial melanin particles. Optionally, at least a portion of each of the artificial melanin particles and/or porous artificial melanin particles comprise a composition according to embodiments disclosed herein throughout. Optionally, the wound comprises inflammation and the step of facilitating comprises reducing the inflammation as a consequence of the extracellular artificial melanin material quenching the extracellular free radical species.

Aspects disclosed herein include a method for treatment of a subject, the method comprising: administering a melanin formulation to a wound of the subject, the wound comprising damaged tissue and extracellular free radical species; wherein the melanin formulation comprises an artificial melanin material; wherein the step of administering comprises providing the artificial melanin material extracellularly at the wound; facilitating tissue healing in the wound via the administered extracellular artificial melanin material; wherein at least a portion of the artificial melanin particles are extracellular at the wound during the step of facilitating tissue healing; and wherein the step of facilitating tissue healing comprises quenching at least the extracellular free radical species by the extracellular artificial melanin material. Optionally, the artificial melanin material comprises porous artificial melanin material, artificial melanin particles, and/or porous artificial melanin particles. Optionally, at least a portion of each of the artificial melanin particles and/or porous artificial melanin particles comprise a composition according to embodiments disclosed herein throughout. Optionally, the wound comprises inflammation and the step of facilitating comprises reducing the inflammation as a consequence of the extracellular artificial melanin material quenching the extracellular free radical species. Optionally, the melanin formulation is administered topically.

Aspects disclosed herein include a method for treatment of a subject, the method comprising: administering a melanin formulation to a region of the subject, the region comprising damaged tissue and an inflammation; wherein the melanin formulation comprises melanin particles; facilitating tissue healing in the region as a result of the presence of administered melanin particles.

Aspects disclosed herein include a method for treatment of a subject, the method comprising: administering a melanin formulation to a region of the subject, the region comprising damaged tissue; wherein the melanin formulation comprises melanin particles; and facilitating tissue healing in the region as a result of the presence of the melanin particles; wherein the step of facilitating comprises the melanin particles quenching reactive oxygenated species in the region. Optionally, extracellular melanin particles of the administered formulation quench extracellular reactive oxygenated species in the region. Optionally, the administration is topical and tissue healing comprises healing skin tissue.

Aspects disclosed herein include a method for treatment of a subject, the method comprising: administering a melanin formulation to a wound of the subject; wherein the melanin formulation comprises melanin particles; and facilitating tissue healing in the wound as a result of the presence of the melanin particles; wherein the step of facilitating comprises the melanin particles quenching reactive oxygenated species in the wound. Optionally, extracellular melanin particles of the administered formulation quench extracellular reactive oxygenated species in the wound. Optionally, the administration is topical and tissue healing comprises healing skin tissue.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIGS. 1A-1G: Characterization of high- and low-surface area synthetic melanin particles, corresponding to embodiments herein of artificial melanin materials. FIGS. 1A-1B: TEM and SEM micrographs of HSA-SMP, respectively. Scale bars 1 micron. FIGS. 1C-1D: TEM and SEM micrographs of LSA-SMP, respectively. Scale bars 1 micron. FIGS. 1E-1F: H&E staining images of mouse skin sections with solid PDA and porous PDA, respectively, shown residing on the surface of the skin. Scale bars 100 nm. FIG. 1G: 2,2-diphenyl-1-picrylhydrazyl (DPPH) radical scavenging activity of antioxidants.

FIGS. 2A-2D: SMP treatment improves skin healing after nitrogen-mustard (NM) injury, an exemplary damaged skin according to embodiments herein. FIG. 2A: Representative images of the wounds on days 1 through 5. FIG. 2B: Wound area reduction. n=11-18 mice per group; *p<0.05; ***p<0.001. FIG. 2C: Bi-fold skin thickness measurements for vehicle (circle), LSA-SMP (square), and HSA-SMP (triangle). n=9-10 mice per group; *p<0.03, ***p<0.0003, ****p<0.0001. FIG. 2D: Time to eschar detachment for vehicle (circle), LSA-SMP (square), and HSA-SMP (triangle). n=9-10 mice per group; *p<0.05, ***p<0.001.

FIGS. 3A-3C: PDA nanoparticle, corresponding to embodiments herein of an artificial melanin material, treatment increases SOD activity after NM injury, according to embodiments herein. FIG. 3A: 24 hours, n=. FIG. 3B: 48 hours, n=. FIG. 3C: 72 hours, n=. * p<0.05; ** p<0.01: *** p<0.001.

FIG. 4A: TaqMan mouse immune array results. Genes significantly downregulated in the HSA-SMP-treated group are shown (all p<0.05). The vehicle-only group was used as a reference, corresponding to absence of artificial melanin material. See also Table 1 and Table 2. Gzmb values in the non-treated group were below detection level. FIGS. 4B-4E: SMP treatment downregulates pro-inflammatory signaling after NM-induced injury. FIG. 4B: Western blotting analysis of ERK1/2 phosphorylation 24 hours after injury, n=3. FIG. 4C: Densitometric quantification of data shown in panel A. FIGS. 4D-4E: Expression of Mmp-9 after 48 (FIG. 4D, n=) and 72 (FIG. 4E, n=5) hours. *p<0.05, **p<0.01. FIGS. 4F-4G: SMP treatment downregulates pro-apoptotic signaling after NM-induced injury, (FIG. 4F) TUNEL stained image. FIG. 4G: Mean fluorescence intensity of TUNEL staining.

FIG. 5: Inhibition of Cu/Zn SOD abrogates the effect of SMP on skin healing after NM injury. (A) Representative images of the wounds on different days. (B) Bi-fold skin thickness measurements, n=; (C) Wound area reduction, n=mice per group (D) TUNEL staining, (E) SOD activity: inhibition of Cu/Zn SOD prevents PDA NP from rescuing SOD activity.

FIGS. 6A-6C: Staining images (FIG. 6A) including lower and higher magnification images for damaged skin with nitrogen-mustard (NM) induced injury after treatment with the vehicle/control (i.e., absence of melanin formulation having artificial melanin material), Low Surface Area Synthetic Melanin Particles (LSA-SMP), and with High Surface Area Synthetic Melanin Particles (HSA-SMP) (porous artificial melanin particles), showing that PDA nanoparticle treatment alleviates (facilitates healing) NM-induced damage in human skin explants. FIGS. 6B and 6C show densitometric quantification of data for two important inflammatory factors, CCL20 in FIG. 6B and CXCL8 in FIG. 6C, from human skin explants data. FIGS. 6B and 6C confirms that artificial melanin material disclosed herein downregulates these inflammatory factors.

FIGS. 7A-7D: High Surface Area Synthetic Melanin Particles (HSA-SMP) (lighter gray) and Low Surface Area Synthetic Melanin Particles (LSA-SMP) (darker gray/black) characterization, according to embodiments herein. FIG. 7A: N₂ adsorption (solid markers) and desorption (open markers) and pore size distribution determined using density functional theory (DFT). FIG. 7B: Pore size distribution of HSA-LSA determined using density functional theory (DFT). FIG. 7C: Dynamic light scattering. FIG. 7D: Ultraviolet visible spectroscopy.

FIGS. 8A-8B: DPPH radical scavenging activity of antioxidants of High Surface Area Synthetic Melanin Particles (HSA-SMP) (solid markers) and Low Surface Area Synthetic Melanin Particles (LSA-SMP) (open markers), according to embodiments herein. FIG. 8A: Multiple samples. FIG. 8B: Scavenging activity cycles. DPPH assay was performed on particles (cycle 1) and washed with water after. Particles were left in water for one week and DPPH assay was performed again (cycle 2).

FIGS. 9A-9C: Synthetic Melanin Particle treatment improves skin healing after ultraviolet (UV) injury. FIG. 9A: UV wound injury images on mouse skin for days 1-5 for vehicle, LSA SMP, and HSA SMP. FIG. 9B: Wound area reduction. n=4-5 mice per group; *p<0.05; ***p<0.005; ***p<0.0005. FIG. 9C: Skin thickness % of UV with vehicle (circle), LSA SMP (square), and HSA SMP (triangle). n=4-5 mice per group; *p<0.05; ***p<0.005; ***p<0.0005.

FIGS. 10A-10B: In embodiments, SMP treatment does not regulate catalase and thioredoxin activity after nitrogen-mustard (NM) induced injury. FIG. 10A: Thioredoxin reductase activity. FIG. 10B: Catalase activity. n=4-10 mice per group.

FIG. 11: TaqMan mouse immune array results. *p<0.05

### STATEMENTS REGARDING CHEMICAL COMPOUNDS AND NOMENCLATURE

In general, the terms and phrases used herein have their art-recognized meaning, which can be found by reference to standard texts, journal references and contexts known to those skilled in the art. The following definitions are provided to clarify their specific use in the context of the invention.

The term “damaged skin” refers to a region of skin of a living subject, the region comprising damaged skin tissue. The damaged skin may optionally comprise free radical species, including extracellular free radical species. The damaged skin may optionally comprise inflammation. The damaged skin may optionally be a closed wound. The damaged skin may optionally include a stratum corneum. The damaged skin may optionally include one or more visible blisters, microscopic vesicles, and separation of the epidermis from the dermis. The damaged skin thereof may optionally include thermally-induced damage, chemically-induced damage, UV-induced damage, mechanical-friction damage, infection cellulitis-induced damage, and/or radiation-induced damage.

The term “closed wound” is intended to be consistent with the term of art, particularly in the fields of biomedical sciences, generally referring to a wound where there remnants of superficial skin attached. Generally, a closed wound may optionally, but not necessarily, comprise one or more small openings and/or compromised focal areas while having remnants of superficial skin attached. For example, generally, a lesion formed by excision biopsy of skin may be characterized as an open wound rather than a closed wound. For example, an open ulcer bed may be characterized as an open wound rather than a closed wound.

The term “therapeutic extracellular activity” refers to a therapeutic activity or therapeutic function that occurs extracellularly and/or involves at least one extracellular species directly performing or directly participating in the activity. The term “therapeutic extracellular activity” is intended to be understood by one of skill in art of biomedical sciences. For example, a therapeutic extracellular activity may refer to an extracellular melanin material performing or participating in a therapeutic activity. For example, a therapeutic extracellular activity may refer to an extracellular species being acted upon to therapeutic effect. For example, a therapeutic extracellular activity may refer to an extracellular melanin material performing or participating in a therapeutic activity upon or with an extracellular species, such as but not limited to extracellular free radical species, extracellular inflammatory factor(s), and/or extracellular enzymatic factor(s). A therapeutic activity or function is an activity or function that has a therapeutic or pharmaceutical effect or benefit, such as for treatment or amelioration of an injury, wound, tissue damage, disease, pathology, or condition. A therapeutic activity or function may comprise one or more chemical and/or physical processes, such as one or more chemical reactions or transformations, one or more physical transformations, covalent or non-covalent association or interaction between species, adsorption, etc. therapeutic activity may include, for example, processes such as, but not limited to, quenching or scavenging, adsorption, regulation such as downregulation of gene expression, inhibition of activity or function of one or more species such as but not limited to proteins, enzymes, or gene expression factors, and any combination thereof. The term “therapeutic intracellular activity” refers to a therapeutic activity or therapeutic function that occurs intracellular and/or involves at least one intracellular species directly performing or directly participating in the activity. A therapeutic activity or function may optionally be both a therapeutic extracellular activity and a therapeutic intracellular activity if both extracellular and intracellular species are involved or participating, for example.

The term “inflammatory factor” refers to a factor, as the term is recognized in the art, particularly biomedical sciences, associated with inflammation. An inflammatory factor may include, but is not limited to, proteins, genes, enzymes, and/or other factors associated with inflammation. The term “enzymatic factor” refers to a factor, as the term is recognized in the art, particularly biomedical sciences, associated with enzyme activity, optionally including enzymes associated with inflammation. Inflammatory factors and/or enzymatic factors may include, but are not limited to, TNFα, iNOS, MMP9, ERK1/2, p38, JNK, one or more factors associated with downregulation of pro-inflammatory signaling, one or more factors regulating expression of one or more genes associated with inflammation, one or more factors regulating expression of one or more genes associated with apoptosis, one or more factors regulating expression of MMP9, one or more proteins associated with the MAPK/ERK pathway, one or more enzymes associated with the MAPK/ERK pathway, or any combination of these. The term “apoptosis factor” refers to a factor, as the term is recognized in the art, particularly biomedical sciences, associated with cellular apoptosis.

The term “wound” refers to a region of a living subject having damaged tissue. The wound may optionally comprise free radical species, including extracellular free radical species. The wound may optionally comprise inflammation. The wound optionally is a wound of skin tissue or optionally includes damaged skin tissue. The wound may optionally include a stratum corneum. The wound may optionally include one or more blisters. The wound or the damaged tissue thereof may optionally include thermally-induced damage, chemically-induced damage, UV-induced damage, mechanical-friction damage, infection cellulitis-induced damage, and/or radiation-induced damage.

The term “free radical species” is intended to be consistent with the term as recognized by one of skill in the art of chemistry or biochemistry. A free radical species is generally a molecular species capable of independent existence and which comprise one or more unpaired electrons. Free radical species may include those that are mutagenic, carcinogenic, cause production of DNA strand breaks, and/or create DNA-protein crosslinks. Exemplary free radical species, include but are not limited to, reactive oxygenated species such as reactive oxygen species (ROS).

The terms “quenching” and “scavenging” are used interchangeably herein and refer to a process of quenching or scavenging free radical species consistent with the art of chemistry or biochemistry. Generally, quenching refers to a process or reaction with or involving a free radical species that results in a conversion/transformation of the free radical species to one or more products that are not free radical species or otherwise the cessation of the existence of the free radical species as a result of the reaction.

The term “non-melanin therapeutic agent” refers to a therapeutic agent that is not and does not itself comprise a melanin material. A therapeutic agent may be, for example, a species such as a compound, molecule, or a moiety that is therapeutically or pharmaceutically active when exposed to a living subject or is capable of treating or managing a condition, such as a disease, in a living subject. For example, a therapeutic agent may be or comprise a small molecule drug, a polymer, a peptide, an amino acid, DNA, or RNA. Optionally, for example, a therapeutic agent is one or more therapeutic agents (one or more compounds, molecules, or moieties, etc.) capable of facilitating skin healing.

The term “extracellular” when describing a species or process is intended to be consistent with the art of pharmacology or biochemistry and refers to the described species or process being found or occurring outside of a cell (i.e., not intracellular).

The term “subject” or “patient” refers to a living organism suffering from or having a wound, disease, or condition that can be treated or remediated, at least in part, by administration of a formulation or melanin material as provided herein. Non-limiting examples include humans, other mammals, bovines, rats, mice, dogs, monkeys, goat, sheep, cows, deer, and other non-mammalian animals. In some embodiments, such as some of Aspects 1-61, a subject is human. In some embodiments, such as some of Aspects 1-61, a subject is a mammal. In some embodiments, such as some of Aspects 1-61, a subject is a mouse. In some embodiments, such as some of Aspects 1-61, a subject is an experimental animal. In some embodiments, such as some of Aspects 1-61, a subject is a rat. In some embodiments, such as some of Aspects 1-61, a subject is a test animal.

The term “melanin” generally refers to one or more compounds or materials that function as a pigment, such as when internalized or taken up by a biological cell, for example. It is also noted that melanin is not necessarily taken up by cells. Melanin can be incorporated in or on cell walls in fungi, for example, such as to provide rigidity, defense mechanisms, and more. In another illustrative example, melanin is used by birds, such as where melanin is organized in a matrix of keratin or similar type of biological material, where it can be organized into monolayers or multilayers to provide structural color, warmth, and more. A melanin compound or material may be, but is not limited to, a melanin monomer, a melanin oligomer, a melanin polymer, a melanin nanoparticle, a melanin layer (e.g., a melanin thin film or coating), or other melanin material, for example. For example, melanin nanoparticles internalized by a biological cell function as a pigment in the cell.

The terms “artificial melanin” and “synthetic melanin” are used interchangeably herein and refer to one or more melanin compounds, molecules, or materials, such as melanin monomers, melanin oligomers, or melanin nanoparticles, that are synthesized and are at least partially, or preferably entirely, not derived from or not extracted from a natural source, such as a biological source, a living organism, or a once living organism. The terms “synthetic” and “artificial” are used interchangeably herein when referring to a melanin or a material comprising a melanin. The terms “synthetic melanin nanoparticles” and “artificial melanin nanoparticles” are used interchangeably herein, and are intended to have the same meaning throughout the present disclosure, and refer to nanoparticles formed of artificial melanin, such as artificial melanin monomers and/or artificial melanin oligomers. The terms “synthetic melanin thin film” and “artificial melanin thin film” are used interchangeably herein, and are intended to have the same meaning throughout the present disclosure, and refer to a thin film formed of artificial melanin, such as artificial melanin monomers and/or artificial melanin oligomers. The terms “synthetic melanin layer” and “artificial melanin layer” are used interchangeably herein, and are intended to have the same meaning throughout the present disclosure, and refer to a layer formed of artificial melanin, such as artificial melanin monomers and/or artificial melanin oligomers. An artificial melanin nanoparticle, artificial melanin thin film, artificial melanin layer, and any compound, material, or formulation comprising any of these, comprises artificial melanin monomers, artificial melanin oligomers, and/or artificial melanin polymers. Optionally, an artificial melanin nanoparticle, artificial melanin thin film, artificial melanin layer, and any compound, material, or formulation comprising any of these, consists of or consists essentially of artificial melanin, such as artificial melanin monomers, artificial melanin oligomers, and/or artificial melanin polymers. Optionally, an artificial melanin nanoparticle, artificial melanin thin film, artificial melanin layer, and any compound, material, or formulation comprising any of these, is free (or substantially free) of artificial melanin monomers and comprises artificial melanin oligomers and/or artificial melanin polymers. Preferably, each artificial melanin monomer, artificial melanin oligomer, and artificial melanin polymer of an artificial melanin nanoparticle, artificial melanin thin film, artificial melanin layer, and any compound, material, or formulation comprising any of these, is not bound to, conjugated to, attached to, coated by, encompassed by or chemically otherwise associated with a natural or biological proteinaceous lipid. A natural or biological proteinaceous lipid refers to a naturally or biologically derived lipid or a lipid extracted from a natural or biological source, such as a once living organism, said lipid comprising one or more proteins such as the lipid (plasma) membrane of a melanocyte or melanosome). Optionally, each artificial melanin monomer, artificial melanin oligomer, and artificial melanin polymer of an artificial melanin nanoparticle, artificial melanin thin film, artificial melanin layer, and any compound, material, or formulation comprising any of these, is not bound to, conjugated to, attached to, coated by, encompassed by or otherwise chemically associated with a natural or biological lipid (e.g. a lipid bilayer, lipid membrane or phospholipid compound). A natural or biological lipid refers to a naturally or biologically derived lipid or a lipid extracted from a natural or biological source, such as a once living organism. Optionally, any artificial melanin monomer, artificial melanin oligomer, and artificial melanin polymer of an artificial melanin nanoparticle, artificial melanin thin film, artificial melanin layer, and any compound, material, or formulation comprising any of these, is bound to, conjugated to, attached to, coated by, encompassed by, and/or otherwise associated with a synthetic or artificial lipid or with a synthetic or artificial phospholipid. A synthetic or artificial lipid refers to a synthesized lipid that is not derived from or is not extracted from a natural or biological source, such as a once living organism.

The term “artificial melanin precursor” refers to a compound or material that can form an artificial melanin material after a chemical reaction, such as after a chemical reaction with an oxidation agent. An artificial melanin precursor can be, but is not necessarily, itself a melanin. For example, an artificial melanin precursor can be, but is not necessarily, a melanin monomer. For example, contacting artificial melanin precursors such as melanin monomers with an oxidizing agent can result in oxidative oligomerization (or, polymerization) among the artificial melanin precursors thereby forming artificial melanin material(s).

The term “selenomelanin” refers to melanin comprising selenium. For example, a selenomelanin material comprises selenium. Preferably, a chemical formula of a selenomelanin material comprises selenium (e.g., at least one selenium atom).

In certain embodiments, the term “pheomelanin” refers to a melanin whose chemical formula comprises at least one substituted or unsubstituted benzothiazine, at least one substituted or unsubstituted benzothiazole, at least one substituted or unsubstituted benzoselenazole, at least one substituted or unsubstituted benzoselenazine, at least one derivative of any of these, or any combination of these. In certain embodiments, the term pheomelanin refers to a melanin made from L-DOPA and cysteine, whose chemical formula comprises at least one substituted or unsubstituted benzothiazine, at least one substituted or unsubstituted benzothiazole, at least one substituted or unsubstituted benzoselenazole, at least one substituted or unsubstituted benzoselenazine, at least one derivative of any of these, or any combination of these. In certain embodiments, a selenium pheomelanin refers to a melanin whose chemical formula comprises at least one substituted or unsubstituted benzoselenazole, at least one substituted or unsubstituted benzoselenazine, at least one derivative of any of these, or any combination of these.

In certain embodiments, the term eumelanin refers to a melanin whose chemical formula comprises at least one dihydoxyindole (DHI) (e.g., 5,6-dihydroxyindole), at least one dihydroxyindole-2-carboxylic acid (DHICA) (e.g., 5,6-dihydroxyindole-2-carboxylic acid), or a combination of these.

The term “nanoparticle” as used herein, refers to a physical particle having at least one size characteristic or physical dimension less than less than 1 μm. Preferably, term “nanoparticle” as used herein, refers to a physical particle whose longest size characteristic or physical dimension is less than 1 μm.

The term “size characteristic” refers to a property, or set of properties, of a particle that directly or indirectly relates to a size attribute. According to some embodiments, a size characteristic corresponds to an empirically-derived size characteristic of a particle(s) being detected, such as a size characteristic based on, determined by, or corresponding to data from any technique or instrument that may be used to determine a particle size, such as electron microscope (e.g., SEM and TEM) or a light scattering technique (e.g., DLS). For example, a size characteristic can correspond to a spherical particle exhibiting similar or substantially same properties, such as aerodynamic, hydrodynamic, optical, and/or electrical properties, as the particle(s) being detected). According to some embodiments, a size characteristic corresponds to a physical dimension, such as a cross-sectional size (e.g., length, width, thickness, or diameter).

The term “particles” refers to small solid objects that may be dispersed and/or suspended in a fluid (e.g., liquid). For example, a slurry, a dispersion, and a suspension each include particles in a fluid. The terms “particle” and “particulate” may be used interchangeably. An exemplary particle is an artificial melanin nanoparticle. A plurality of particles may be associated together to form an agglomerate of particles. Generally, the term “particle”, such as “nanoparticle” or “melanin nanoparticle”, refers to an individual particle rather than to an agglomerate of such individual particles.

The term “dispersed” refers to species, such as particles, in a fluid forming a dispersion. As used herein, the term “dispersion” broadly refers to a mixture of one or more chemical species, such as particles, in a fluid, such as the art-recognized meaning of solution, dispersion, and/or suspension. The chemical species, such as particles, dispersed in a dispersion can be referred as a dispersed species. Preferably, a dispersion is a mixture of particles, such as artificial melanin particles, in a liquid, such as a solvent. Preferably, but not necessarily, a dispersion is a homogeneous mixture. In the context of a dispersion, the term “homogeneous” refers to a liquid mixture that appears uniform to the naked eye. In contrast, a heterogenous liquid mixture includes particles that are precipitated from or suspended in the liquid mixture and are large enough to be distinctly identifiable by the naked eye in the liquid mixture. A heterogeneous liquid mixture includes, for example, sedimented and/or sedimenting particles. Preferably, but not necessarily, the term “dispersion” is broadly intended to include solutions and dispersions, such as colloids, which are not heterogenous liquid mixtures. Preferably, but not necessarily, a dispersion is a microscopically homogenous, or uniform, mixture of particles in a liquid, such as a solvent. Preferably, but not necessarily, a dispersion is thermodynamically favored remain stably dispersed or is thermodynamically favored to segregate by sedimentation but wherein sedimentation is kinetically slowed or prevented. Particles, of a dispersion, that are characterized as stably dispersed remain dispersed in the dispersion and do not sediment or precipitate out of the liquid, of the dispersion, for at least 5 hours, preferably at least 12 hours, preferably at least 24 hours, and more preferably at least 1 week, under normal temperature and pressure (NTP) and exposure to air. In embodiments, particles that are not or cannot be dispersed in a fluid refer to particles that form precipitates or sediments upon being mixed in the fluid.

When referring to a material, such as a polymer, being aqueous, the term “aqueous” refers to said material being dispersed, dissolved, or otherwise solvated by water. An “aqueous solution” refers to a solution that comprises water as solvent and one or more solute species dispersed, dissolved, or otherwise solvated by the water. An aqueous process, such as a polymerization, is a process taking place in an aqueous solution. Optionally, but not necessarily, an aqueous solution or an aqueous solvent includes 20 vol. % or less, optionally 15 vol. % or less, optionally 10 vol. % or less, preferably 5 vol. % or less, of a non-water or organic species. Optionally, but not necessarily, an aqueous solution or an aqueous solvent includes 20 vol. % or less, optionally 15 vol. % or less, optionally 10 vol. % or less, preferably 5 vol. % or less, of a non-water liquid.

The term “peak size” size refers to the statistical mode, or peak frequency, of a particle size distribution, or the particle size most commonly found in the particle size distribution. A particle size distribution can be measured using dynamic light scattering, for example.

The term “sphere” as used herein, in the usual and customary sense, refers to a round or substantially round geometrical object in three-dimensional space that is substantially the surface of a completely round ball, analogous to a circular object in two dimensions. A sphere may be defined mathematically as the set of points that are all at the same or substantially all at the same distance r from a given point, but in three-dimensional space, where r is the radius of the mathematical ball and the given point is the center or substantially the center of the mathematical ball. In embodiments, the longest straight line through the ball, connecting two points of the sphere, passes through the center and its length is thus twice the radius; it is a diameter of the ball. A nanosphere is a nanoparticle having a radius of less than 1 μm.

The terms “reactive oxygen species” and “ROS” as used interchangeably herein refer, in the usual and customary sense, to transient species, typically formed during exposure to radiation (e.g., UV irradiation) capable of inducing oxidative decomposition.

The terms “cell” and “biological cell” are used interchangeably are refer to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., Spodoptera) and human cells. A “viable cell” is a living biological cell.

The term “substantially” refers to a property, condition, or value that is within 20%, 10%, within 5%, within 1%, optionally within 0.1%, or is equivalent to a reference property, condition, or value. The term “substantially equal”, “substantially equivalent”, or “substantially unchanged”, when used in conjunction with a reference value describing a property or condition, refers to a value that is within 20%, within 10%, optionally within 5%, optionally within 1%, optionally within 0.1%, or optionally is equivalent to the provided reference value. For example, a diameter is substantially equal to 100 nm (or, “is substantially 100 nm”) if the value of the diameter is within 20%, optionally within 10%, optionally within 5%, optionally within 1%, within 0.1%, or optionally equal to 100 nm. The term “substantially greater”, when used in conjunction with a reference value describing a property or condition, refers to a value that is at least 1%, optionally at least 5%, optionally at least 10%, or optionally at least 20% greater than the provided reference value. The term “substantially less”, when used in conjunction with a reference value describing a property or condition, refers to a value that is at least 1%, optionally at least 5%, optionally at least 10%, or optionally at least 20% less than the provided reference value.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

The terms “treating” or “treatment” as used herein, refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating,” and conjugations thereof, include prevention of an injury, pathology, condition, or disease.

The term “effective amount” as used herein, refers to an amount sufficient to accomplish a stated purpose (e.g. Achieve the effect for which it is administered, treat a disease, reduce one or more symptoms of a disease or condition, and the like). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “administering” as used herein, refers to oral administration, administration as an inhaled aerosol or as an inhaled dry powder, suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compound of the invention can be administered alone or can be co-administered to the patient. Co-administration is meant to include simultaneous or sequential administration of the compound individually or in combination (more than one compound or agent). The compositions of the present invention can be delivered transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293306, 1996; Chonn, Curr. Opin. Biotechnol. 6:698-708, 1995; Qstio, Am. J Hasp. Pharm. 46: 1576-1587, 1989).

The term “contacting” may include allowing two species to react, interact, or physically touch, wherein the two species may be, for example, a pharmaceutical composition as provided herein and a cell. In embodiments contacting includes, for example, allowing a pharmaceutical composition as described herein to interact with a cell or a patient.

The terms “analog” and “analogue” are used interchangeably and are used in accordance with their plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound, including isomers thereof. Accordingly, an analog is a compound that is similar or comparable in function and appearance but not in structure or origin to a reference compound.

Except where otherwise specified, the term “molecular weight” refers to an average molecular weight. Except where otherwise specified, the term “average molecular weight,” refers to number-average molecular weight. Number average molecular weight is defined as the total weight of a sample volume divided by the number of molecules within the sample. As is customary and well known in the art, peak average molecular weight and weight average molecular weight may also be used to characterize the molecular weight of the distribution of polymers within a sample.

The term “weight-average molecular weight” (M_{w}) refers to the average molecular weight defined as the sum of the products of the molecular weight of each polymer molecule (Mᵢ) multiplied by its weight fraction (wᵢ): M_{w}=ΣwᵢMᵢ. As is customary and well known in the art, peak average molecular weight and number average molecular weight may also be used to characterize the molecular weight of the distribution of polymers within a sample.

The term “wt. %” or “wt %” refers to a weight percent, or a mass fraction represented as a percentage by mass. The term “at. %” or “at %” refers to an atomic percent, or an atomic ratio represented as a percentage of a type of atom with respect to total atoms in a given matter, such as a molecule, compound, material, nanoparticle, polymer, dispersion, etc.

The term “oligomerization” refers to a chemical process of converting a monomer or a mixture of monomers into an oligomer. The term “oxidative oligomerization” refers to a chemical process of oligomerization that includes chemical oxidation of one or more monomers to form an oligomer. An oligomerization is a polymerization process, wherein an oligomer is formed as a result of the polymerization.

As used herein, the term “polymer” refers to a molecule composed of repeating structural units connected by covalent chemical bonds often characterized by a number of repeating units, also referred to as base units (e.g., greater than or equal to 2 base units). As used herein, a term “polymer” is inclusive of an “oligomer” (i.e., an oligomer is a polymer; i.e., a polymer is optionally an oligomer). An “oligomer” refers to a molecule composed of repeating structural units, also referred to as base units, connected by covalent chemical bonds often characterized by a number of repeating units less such that the oligomer is a low molecular weight polymer. Preferably, but not necessarily, for example, an oligomer has equal to or less than 100 repeating units. Preferably, but not necessarily, for example, an oligomer has a lower molecular weight less than or equal to 10,000 Da. Oligomers may be the polymerization product of one or more monomer precursors. Polymerization of one or more monomers, or monomer precursors, resulting in formation of an oligomer may be referred to as oligomerization. An oligomer optionally includes 100 or less, 50 or less, 15 or less, 12 or less, 10 or less, or 5 or less repeating units (or, “base units”). An oligomer may be characterized has having a molecular weight of 10,000 Da or less, 5,000 Da or less, 1,000 Da or less, 500 Da or less, or 200 Da or less. A dimer, a trimer, a tetramer, or a pentamer is an oligomer having two, three, four, or five, respectively, repeating units, or base units. Polymers can have, for example, greater than 100 repeating units. Polymers can have, for example, a high molecular weight, such as greater than 10,000 Da, in some embodiments greater than or equal to 50,000 Da or greater than or equal to 100,000 Da. The term polymer includes homopolymers, or polymers consisting essentially of a single repeating monomer subunit. The term polymer also includes copolymers which are formed when two or more different types of monomers are linked in the same polymer. Copolymers may comprise two or more monomer subunits, and include random, block, brush, brush block, alternating, segmented, grafted, tapered and other architectures. Useful polymers include organic polymers or inorganic polymers that may be in amorphous, semi-amorphous, crystalline or semi-crystalline states. Polymer side chains capable of cross linking polymers (e.g., physical cross linking) may be useful for some applications.

An “oligomer” refers to a molecule composed of repeating structural units, also referred to as base units, connected by covalent chemical bonds often characterized by a number of repeating units less than that of a polymer (e.g., equal to or less than 100 repeating units) and a lower molecular weights (e.g. less than or equal to 10,000 Da) than polymers. Oligomers may be the polymerization product of one or more monomer precursors. Polymerization of one or more monomers, or monomer precursors, resulting in formation of an oligomer may be referred to as oligomerization. An oligomer optionally includes 100 or less, 50 or less, 15 or less, 12 or less, 10 or less, or 5 or less repeating units (or, “base units”). An oligomer may be characterized has having a molecular weight of 10,000 Da or less, 5,000 Da or less, 1,000 Da or less, 500 Da or less, or 200 Da or less. A dimer, a trimer, a tetramer, or a pentamer is an oligomer having two, three, four, or five, respectively, repeating units, or base units.

As used herein, the term “group” may refer to a functional group of a chemical compound. Groups of the present compounds refer to an atom or a collection of atoms that are a part of the compound. Groups of the present invention may be attached to other atoms of the compound via one or more covalent bonds. Groups may also be characterized with respect to their valence state. The present invention includes groups characterized as monovalent, divalent, trivalent, etc. valence states.

The term “moiety” refers to a group, such as a functional group, of a chemical compound or molecule. A moiety is a collection of atoms that are part of the chemical compound or molecule. The present invention includes moieties characterized as monovalent, divalent, trivalent, etc. valence states. Generally, but not necessarily, a moiety comprises more than one functional group.

As used herein, the term “substituted” refers to a compound wherein one or more hydrogens is replaced by another functional group, provided that the designated atom's normal valence is not exceeded. An exemplary substituent includes, but is not limited to: a halogen or halide, an alkyl, a cycloalkyl, an aryl, a heteroaryl, an acyl, an alkoxy, an alkenyl, an alkynyl, an alkylaryl, an arylene, a heteroarylene, an alkenylene, a cycloalkenylene, an alkynylene, a hydroxyl (—OH), a carbonyl (RCOR′), a sulfide (e.g., RSR′), a phosphate (ROP(═O)(OH)₂), an azo (RNNR′), a cyanate (ROCN), an amine (e.g., primary, secondary, or tertiary), an imine (RC(═NH)R′), a nitrile (RCN), a pyridinyl (or pyridyl), a diamine, a triamine, an azide, a diimine, a triimine, an amide, a diimide, or an ether (ROR′); where each of R and R′ is independently a hydrogen or a substituted or unsubstituted alkyl group, aryl group, alkenyl group, or a combination of these. Optional substituent functional groups are also described below. In some embodiments, such as some of Aspects 1-61, the term substituted refers to a compound wherein each of more than one hydrogen is replaced by another functional group, such as a halogen group. For example, when the substituent is oxo (i.e., ═O), then two hydrogens on the atom are replaced. The substituent group can be any substituent group described herein. For example, substituent groups can include one or more of a hydroxyl, an amino (e.g., primary, secondary, or tertiary), an aldehyde, a carboxylic acid, an ester, an amide, a ketone, nitro, an urea, a guanidine, cyano, fluoroalkyl (e.g., trifluoromethane), halo (e.g., fluoro), aryl (e.g., phenyl), heterocyclyl or heterocyclic group (i.e., cyclic group, e.g., aromatic (e.g., heteroaryl) or non-aromatic where the cyclic group has one or more heteroatoms), oxo, or combinations thereof. Combinations of substituents and/or variables are permissible provided that the substitutions do not significantly adversely affect synthesis or use of the compound.

As used herein, the term “derivative” refers to a compound wherein an atom or functional group is replaced by another atom or functional group (e.g., a substituent function group as also described below), including, but not limited to: a hydrogen, a halogen or halide, an alkyl, a cycloalkyl, an aryl, a heteroaryl, an acyl, an alkoxy, an alkenyl, an alkynyl, an alkylaryl, an arylene, a heteroarylene, an alkenylene, a cycloalkenylene, an alkynylene, a hydroxyl (—OH), a carbonyl (RCOR′), a sulfide (e.g., RSR′), a phosphate (ROP(═O)(OH)₂), an azo (RNNR′), a cyanate (ROCN), an amine (e.g., primary, secondary, or tertiary), an imine (RC(═NH)R′), a nitrile (RCN), a pyridinyl (or pyridyl), a diamine, a triamine, an azide, a diimine, a triimine, an amide, a diimide, or an ether (ROR′); where each of R and R′ is independently a hydrogen or a substituted or unsubstituted alkyl group, aryl group, alkenyl group, or a combination of these. Optional substituent functional groups are also described below. Preferably, the term “derivative” refers to a compound wherein one or two atoms or functional groups are independently replaced by another atom or functional group. Optionally, the term derivative does not refer to or include replacement of a chalcogen atom (S, Se) that is a member of a heterocyclic group. Optionally, and unless otherwise stated, the term derivative does not refer to or include replacement of a chalcogen atom (S, Se) nor a N (nitrogen) where the chalcogen atom and the N are members same heterocyclic group. Optionally, but not necessarily, the term derivative does not include breaking a ring structure, replacement of a ring member, or removal of a ring member.

As is customary and well known in the art, hydrogen atoms in formula, are not always explicitly shown, for example, hydrogen atoms bonded to the carbon atoms of aromatic, heteroaromatic, and alicyclic rings are not always explicitly shown. The structures provided herein, for example in the context of the description of formula and schematics and structures in the drawings, are intended to convey to one of reasonable skill in the art the chemical composition of compounds of the methods and compositions of the invention, and as will be understood by one of skill in the art, the structures provided do not indicate the specific positions and/or orientations of atoms and the corresponding bond angles between atoms of these compounds.

As used herein, the terms “alkylene” and “alkylene group” are used synonymously and refer to a divalent group derived from an alkyl group as defined herein. The invention includes compounds having one or more alkylene groups. Alkylene groups in some compounds function as linking and/or spacer groups. Compounds of the invention may have substituted and/or unsubstituted C₁-C₂₀ alkylene, C₁-C₁₀ alkylene and C₁-C₅ alkylene groups, for example, as one or more linking groups (e.g. L¹-L⁶).

As used herein, the terms “cycloalkylene” and “cycloalkylene group” are used synonymously and refer to a divalent group derived from a cycloalkyl group as defined herein. The invention includes compounds having one or more cycloalkylene groups. Cycloalkyl groups in some compounds function as linking and/or spacer groups. Compounds of the invention may have substituted and/or unsubstituted C₃-C₂₀ cycloalkylene, C₃-C₁₀ cycloalkylene and C₃-C₅ cycloalkylene groups, for example, as one or more linking groups (e.g. L¹-L⁶).

As used herein, the terms “arylene” and “arylene group” are used synonymously and refer to a divalent group derived from an aryl group as defined herein. The invention includes compounds having one or more arylene groups. In some embodiments, such as some of Aspects 1-61, an arylene is a divalent group derived from an aryl group by removal of hydrogen atoms from two intra-ring carbon atoms of an aromatic ring of the aryl group. Arylene groups in some compounds function as linking and/or spacer groups. Arylene groups in some compounds function as chromophore, fluorophore, aromatic antenna, dye and/or imaging groups. Compounds of the invention include substituted and/or unsubstituted C₃-C₃₀ arylene, C₃-C₂₀ arylene, C₃-C₁₀ arylene and C₁-C₅ arylene groups, for example, as one or more linking groups (e.g. L¹-L⁶).

As used herein, the terms “heteroarylene” and “heteroarylene group” are used synonymously and refer to a divalent group derived from a heteroaryl group as defined herein. The invention includes compounds having one or more heteroarylene groups. In an embodiment, a heteroarylene is a divalent group derived from a heteroaryl group by removal of hydrogen atoms from two intra-ring carbon atoms or intra-ring nitrogen atoms of a heteroaromatic or aromatic ring of the heteroaryl group. Heteroarylene groups in some compounds function as linking and/or spacer groups. Heteroarylene groups in some compounds function as chromophore, aromatic antenna, fluorophore, dye and/or imaging groups. Compounds of the invention include substituted and/or unsubstituted C₃-C₃₀ heteroarylene, C₃-C₂₀ heteroarylene, C₁-C₁₀ heteroarylene and C₃-C₅ heteroarylene groups, for example, as one or more linking groups (e.g. L¹-L⁶).

As used herein, the terms “alkenylene” and “alkenylene group” are used synonymously and refer to a divalent group derived from an alkenyl group as defined herein. The invention includes compounds having one or more alkenylene groups. Alkenylene groups in some compounds function as linking and/or spacer groups. Compounds of the invention include substituted and/or unsubstituted C₂-C₂₀ alkenylene, C₂-C₁₀ alkenylene and C₂-C₅ alkenylene groups, for example, as one or more linking groups (e.g. L¹-L⁶).

As used herein, the terms “cylcoalkenylene” and “cylcoalkenylene group” are used synonymously and refer to a divalent group derived from a cylcoalkenyl group as defined herein. The invention includes compounds having one or more cylcoalkenylene groups. Cycloalkenylene groups in some compounds function as linking and/or spacer groups. Compounds of the invention include substituted and/or unsubstituted C₃-C₂₀ cylcoalkenylene, C₃-C₁₀ cylcoalkenylene and C₃-C₅ cylcoalkenylene groups, for example, as one or more linking groups (e.g. L¹-L⁶).

As used herein, the terms “alkynylene” and “alkynylene group” are used synonymously and refer to a divalent group derived from an alkynyl group as defined herein. The invention includes compounds having one or more alkynylene groups. Alkynylene groups in some compounds function as linking and/or spacer groups. Compounds of the invention include substituted and/or unsubstituted C₂-C₂₀ alkynylene, C₂-C₁₀ alkynylene and C₂-C₅ alkynylene groups, for example, as one or more linking groups (e.g. L¹-L⁶).

As used herein, the term “halo” refers to a halogen group such as a fluoro (—F), chloro (—CI), bromo (—Br), iodo (—I) or astato (—At).

The term “heterocyclic” refers to ring structures containing at least one other kind of atom, in addition to carbon, in the ring. Examples of such heteroatoms include nitrogen, oxygen and sulfur. Heterocyclic rings include heterocyclic alicyclic rings and heterocyclic aromatic rings. Examples of heterocyclic rings include, but are not limited to, pyrrolidinyl, piperidyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, triazolyl and tetrazolyl groups. Atoms of heterocyclic rings can be bonded to a wide range of other atoms and functional groups, for example, provided as substituents.

The term “carbocyclic” refers to ring structures containing only carbon atoms in the ring. Carbon atoms of carbocyclic rings can be bonded to a wide range of other atoms and functional groups, for example, provided as substituents.

The term “alicyclic ring” refers to a ring, or plurality of fused rings, that is not an aromatic ring. Alicyclic rings include both carbocyclic and heterocyclic rings.

The term “aromatic ring” refers to a ring, or a plurality of fused rings, that includes at least one aromatic ring group. The term aromatic ring includes aromatic rings comprising carbon, hydrogen and heteroatoms. Aromatic ring includes carbocyclic and heterocyclic aromatic rings. Aromatic rings are components of aryl groups.

The term “fused ring” or “fused ring structure” refers to a plurality of alicyclic and/or aromatic rings provided in a fused ring configuration, such as fused rings that share at least two intra ring carbon atoms and/or heteroatoms.

As used herein, the term “alkoxyalkyl” refers to a substituent of the formula alkyl-O-alkyl.

As used herein, the term “polyhydroxyalkyl” refers to a substituent having from 2 to 12 carbon atoms and from 2 to 5 hydroxyl groups, such as the 2,3-dihydroxypropyl, 2,3,4-trihydroxybutyl or 2,3,4,5-tetrahydroxypentyl residue.

As used herein, the term “polyalkoxyalkyl” refers to a substituent of the formula alkyl-(alkoxy)ₙ-alkoxy wherein n is an integer from 1 to 10, preferably 1 to 4, and more preferably for some embodiments 1 to 3.

Amino acids include glycine, alanine, valine, leucine, isoleucine, methionine, proline, phenylalanine, tryptophan, asparagine, glutamine, glycine, serine, threonine, serine, rhreonine, asparagine, glutamine, tyrosine, cysteine, lysine, arginine, histidine, aspartic acid and glutamic acid. As used herein, reference to “a side chain residue of a natural α-amino acid” specifically includes the side chains of the above-referenced amino acids. Peptides and peptide moieties, as used and described herein, comprise two or more amino acid groups connected via peptide bonds.

Amino acids and amino acid groups refer to naturally-occurring amino acids, unnatural (non-naturally occurring) amino acids, and/or combinations of these. Naturally-occurring amino acids are those encoded by the genetic code, as well as those amino acids that are later modified, e.g., hydroxyproline, γ-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid. Amino acids may be referred to herein by either the commonly known three letter symbols or by the one-letter symbols recommended by the IUPAC-IUB Biochemical Nomenclature Commission.

The terms “monomer unit,” “repeating monomer unit,” “repeating unit,” and “polymerized monomer” can be used interchangeably and refer to a monomeric portion of a polymer described herein which is derived from or is a product of polymerization of one individual “monomer” or “polymerizable monomer.” Each individual monomer unit of a polymer is derived from or is a product of polymerization of one polymerizable monomer. Each individual “monomer unit” or “repeating unit” of a polymer comprises one (polymerized) polymer backbone group. For example, in a polymer that comprises monomer units X and Y arranged as X-Y-X-Y-X-Y-X-Y (where each X is identical to each other X and each Y is identical to each other Y), each X and each Y is independently can be referred to as a repeating unit or monomer unit.

Alkyl groups include straight-chain, branched and cyclic alkyl groups. Alkyl groups include those having from 1 to 30 carbon atoms. Alkyl groups include small alkyl groups having 1 to 3 carbon atoms. Alkyl groups include medium length alkyl groups having from 4-10 carbon atoms. Alkyl groups include long alkyl groups having more than 10 carbon atoms, particularly those having 10-30 carbon atoms. The term cycloalkyl specifically refers to an alky group having a ring structure such as ring structure comprising 3-30 carbon atoms, optionally 3-20 carbon atoms and optionally 2-10 carbon atoms, including an alkyl group having one or more rings. Cycloalkyl groups include those having a 3-, 4-, 5-, 6-, 7-, 8-, 9- or 10-member carbon ring(s) and particularly those having a 3-, 4-, 5-, 6-, 7-, or 8-member ring(s). The carbon rings in cycloalkyl groups can also carry alkyl groups. Cycloalkyl groups can include bicyclic and tricycloalkyl groups. Alkyl groups are optionally substituted. Substituted alkyl groups include among others those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl groups, such as alkyl groups having one or more hydrogens replaced with one or more fluorine atoms. An alkoxy group is an alkyl group that has been modified by linkage to oxygen and can be represented by the formula R—O and can also be referred to as an alkyl ether group. Examples of alkoxy groups include, but are not limited to, methoxy, ethoxy, propoxy, butoxy and heptoxy. Alkoxy groups include substituted alkoxy groups wherein the alky portion of the groups is substituted as provided herein in connection with the description of alkyl groups. As used herein MeO— refers to CH₃O—. Compositions of some embodiments of the invention comprise alkyl groups as terminating groups, such as polymer backbone terminating groups and/or polymer side chain terminating groups. Substituted alkyl groups may include substitution to incorporate one or more silyl groups, for example wherein one or more carbons are replaced by Si.

Alkenyl groups include straight-chain, branched and cyclic alkenyl groups. Alkenyl groups include those having 1, 2 or more double bonds and those in which two or more of the double bonds are conjugated double bonds. Alkenyl groups include those having from 2 to 20 carbon atoms. Alkenyl groups include small alkenyl groups having 2 to 3 carbon atoms. Alkenyl groups include medium length alkenyl groups having from 4-10 carbon atoms. Alkenyl groups include long alkenyl groups having more than 10 carbon atoms, particularly those having 10-20 carbon atoms. Cycloalkenyl groups include those in which a double bond is in the ring or in an alkenyl group attached to a ring. The term cycloalkenyl specifically refers to an alkenyl group having a ring structure, including an alkenyl group having a 3-, 4-, 5-, 6-, 7-, 8-, 9- or 10-member carbon ring(s) and particularly those having a 3-, 4-, 5-, 6- or 7-member ring(s). The carbon rings in cycloalkenyl groups can also carry alkyl groups. Cycloalkenyl groups can include bicyclic and tricyclic alkenyl groups. Alkenyl groups are optionally substituted. Substituted alkenyl groups include among others those which are substituted with alkyl or aryl groups, which groups in turn can be optionally substituted. Specific alkenyl groups include ethenyl, prop-1-enyl, prop-2-enyl, cycloprop-1-enyl, but-1-enyl, but-2-enyl, cyclobut-1-enyl, cyclobut-2-enyl, pent-1-enyl, pent-2-enyl, branched pentenyl, cyclopent-1-enyl, hex-1-enyl, branched hexenyl, cyclohexenyl, all of which are optionally substituted. Substituted alkenyl groups include fully halogenated or semihalogenated alkenyl groups, such as alkenyl groups having one or more hydrogens replaced with one or more fluorine atoms, chlorine atoms, bromine atoms and/or iodine atoms. Substituted alkenyl groups include fully fluorinated or semifluorinated alkenyl groups, such as alkenyl groups having one or more hydrogen atoms replaced with one or more fluorine atoms. Compositions of some embodiments of the invention comprise alkenyl groups as terminating groups, such as polymer backbone terminating groups and/or polymer side chain terminating groups.

Aryl groups include groups having one or more 5-, 6- 7-, or 8-member aromatic rings, including heterocyclic aromatic rings. The term heteroaryl specifically refers to aryl groups having at least one 5-, 6- 7-, or 8-member heterocyclic aromatic rings. Aryl groups can contain one or more fused aromatic rings, including one or more fused heteroaromatic rings, and/or a combination of one or more aromatic rings and one or more nonaromatic rings that may be fused or linked via covalent bonds. Heterocyclic aromatic rings can include one or more N, O, or S atoms in the ring. Heterocyclic aromatic rings can include those with one, two or three N atoms, those with one or two O atoms, and those with one or two S atoms, or combinations of one or two or three N, O or S atoms. Aryl groups are optionally substituted. Substituted aryl groups include among others those that are substituted with alkyl or alkenyl groups, which groups in turn can be optionally substituted. Specific aryl groups include phenyl, biphenyl groups, pyrrolidinyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, and naphthyl groups, all of which are optionally substituted. Substituted aryl groups include fully halogenated or semihalogenated aryl groups, such as aryl groups having one or more hydrogens replaced with one or more fluorine atoms, chlorine atoms, bromine atoms and/or iodine atoms. Substituted aryl groups include fully fluorinated or semifluorinated aryl groups, such as aryl groups having one or more hydrogens replaced with one or more fluorine atoms. Aryl groups include, but are not limited to, aromatic group-containing or heterocylic aromatic group-containing groups corresponding to any one of the following: benzene, naphthalene, naphthoquinone, diphenylmethane, fluorene, anthracene, anthraquinone, phenanthrene, tetracene, tetracenedione, pyridine, quinoline, isoquinoline, indoles, isoindole, pyrrole, imidazole, oxazole, thiazole, pyrazole, pyrazine, pyrimidine, purine, benzimidazole, furans, benzofuran, dibenzofuran, carbazole, acridine, acridone, phenanthridine, thiophene, benzothiophene, dibenzothiophene, xanthene, xanthone, flavone, coumarin, azulene or anthracycline. As used herein, a group corresponding to the groups listed above expressly includes an aromatic or heterocyclic aromatic group, including monovalent, divalent and polyvalent groups, of the aromatic and heterocyclic aromatic groups listed herein are provided in a covalently bonded configuration in the compounds of the invention at any suitable point of attachment. In embodiments, aryl groups contain between 5 and 30 carbon atoms. In embodiments, aryl groups contain one aromatic or heteroaromatic six-member ring and one or more additional five- or six-member aromatic or heteroaromatic ring. In embodiments, aryl groups contain between five and eighteen carbon atoms in the rings. Aryl groups optionally have one or more aromatic rings or heterocyclic aromatic rings having one or more electron donating groups, electron withdrawing groups and/or targeting ligands provided as substituents. Compositions of some embodiments of the invention comprise aryl groups as terminating groups, such as polymer backbone terminating groups and/or polymer side chain terminating groups.

Arylalkyl groups are alkyl groups substituted with one or more aryl groups wherein the alkyl groups optionally carry additional substituents and the aryl groups are optionally substituted. Specific alkylaryl groups are phenyl-substituted alkyl groups, e.g., phenylmethyl groups. Alkylaryl groups are alternatively described as aryl groups substituted with one or more alkyl groups wherein the alkyl groups optionally carry additional substituents and the aryl groups are optionally substituted. Specific alkylaryl groups are alkyl-substituted phenyl groups such as methylphenyl. Substituted arylalkyl groups include fully halogenated or semihalogenated arylalkyl groups, such as arylalkyl groups having one or more alkyl and/or aryl groups having one or more hydrogens replaced with one or more fluorine atoms, chlorine atoms, bromine atoms and/or iodine atoms. Compositions of some embodiments of the invention comprise arylalkyl groups as terminating groups, such as polymer backbone terminating groups and/or polymer side chain terminating groups.

As to any of the groups described herein which contain one or more substituents, it is understood that such groups do not contain any substitution or substitution patterns which are sterically impractical and/or synthetically non-feasible. In addition, the compounds of this invention include all stereochemical isomers arising from the substitution of these compounds. Optional substitution of alkyl groups includes substitution with one or more alkenyl groups, aryl groups or both, wherein the alkenyl groups or aryl groups are optionally substituted. Optional substitution of alkenyl groups includes substitution with one or more alkyl groups, aryl groups, or both, wherein the alkyl groups or aryl groups are optionally substituted. Optional substitution of aryl groups includes substitution of the aryl ring with one or more alkyl groups, alkenyl groups, or both, wherein the alkyl groups or alkenyl groups are optionally substituted.

Optional substituents for any alkyl, alkenyl and aryl group includes substitution with one or more of the following substituents, among others:
halogen, including fluorine, chlorine, bromine or iodine;
pseudohalides, including —CN;
—COOR where R is a hydrogen or an alkyl group or an aryl group and more specifically where R is a methyl, ethyl, propyl, butyl, or phenyl group all of which groups are optionally substituted;
—COR where R is a hydrogen or an alkyl group or an aryl group and more specifically where R is a methyl, ethyl, propyl, butyl, or phenyl group all of which groups are optionally substituted;
—CON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;
—OCON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;
—N(R)₂ where each R, independently of each other R, is a hydrogen, or an alkyl group, or an acyl group or an aryl group and more specifically where R is a methyl, ethyl, propyl, butyl, phenyl or acetyl group, all of which are optionally substituted; and where R and R can form a ring which can contain one or more double bonds and can contain one or more additional carbon atoms;
—SR, where R is hydrogen or an alkyl group or an aryl group and more specifically where R is hydrogen, methyl, ethyl, propyl, butyl, or a phenyl group, which are optionally substituted;
—SO₂R, or —SOR where R is an alkyl group or an aryl group and more specifically where R is a methyl, ethyl, propyl, butyl, or phenyl group, all of which are optionally substituted;
—OCOOR where R is an alkyl group or an aryl group;
—SO₂N(R)₂ where each R, independently of each other R, is a hydrogen, or an alkyl group, or an aryl group all of which are optionally substituted and wherein R and R can form a ring which can contain one or more double bonds and can contain one or more additional carbon atoms; and

—OR where R is H, an alkyl group, an aryl group, or an acyl group all of which are optionally substituted. In a particular example R can be an acyl yielding —OCOR″ where R″ is a hydrogen or an alkyl group or an aryl group and more specifically where R″ is methyl, ethyl, propyl, butyl, or phenyl groups all of which groups are optionally substituted.

Specific substituted alkyl groups include haloalkyl groups, particularly trihalomethyl groups and specifically trifluoromethyl groups. Specific substituted aryl groups include mono-, di-, tri, tetra- and pentahalo-substituted phenyl groups; mono-, di-, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups; and methoxyphenyl groups, particularly 4-methoxyphenyl groups.

As to any of the above groups which contain one or more substituents, it is understood that such groups do not contain any substitution or substitution patterns which are sterically impractical and/or synthetically non-feasible.

Many of the molecules disclosed herein contain one or more ionizable groups. Ionizable groups include groups from which a proton can be removed (e.g., —COOH) or added (e.g., amines) and groups that can be quaternized (e.g., amines). All possible ionic forms of such molecules and salts thereof are intended to be included individually in the disclosure herein. With regard to salts of the compounds herein, one of ordinary skill in the art can select from among a wide variety of available counterions that are appropriate for preparation of salts of this invention for a given application. In specific applications, the selection of a given anion or cation for preparation of a salt can result in increased or decreased solubility of that salt.

The compounds of this invention can contain one or more chiral centers. Accordingly, this invention is intended to include racemic mixtures, diastereomers, enantiomers, tautomers and mixtures enriched in one or more stereoisomer. The scope of the invention as described and claimed encompasses the racemic forms of the compounds as well as the individual enantiomers and non-racemic mixtures thereof.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another. It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety, functional group, atom, ion, unpaired electron, or other chemical species to the represented molecule, compound, or chemical formula. For example, in the formula

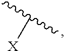

“X” represents a molecule or compound, the symbol “ [symbol] ” denotes a point of attachment of a chemical moiety, functional group, atom, ion, unpaired electron, or other chemical species to X (where X corresponds to the represented molecule, compound, or chemical formula) via covalent bonding. As used herein, the various functional groups represented will be understood to have a point of attachment at the functional group having the hyphen or dash (—) or a dash used in combination with an asterisk (*). In other words, in the case of —CH₂CH₂CH₃ or —CH₂CH₂CH₃, it will be understood that the point of attachment is the CH₂ group at the far left. If a group is recited without an asterisk or a dash, then the attachment point is indicated by the plain and ordinary meaning of the recited group.

Where substituent groups are specified by their conventional chemical formulae, written from left to right, they equally encompass the chemically identical substituents that would result from writing the structure from right to left, e.g., —CH₂O— is equivalent to —OCH₂—.

The term “±” refers to an inclusive range of values, such that “X±Y,” wherein each of X and Y is independently a number, refers to an inclusive range of values selected from the range of X−Y to X+Y. In the cases of “X±Y” wherein Y is a percentage (e.g., 1.0±20%), the inclusive range of values is selected from the range of X−Z to X+Z, wherein Z is equal to X·(Y/100). For example, 1.0±20% refers to the inclusive range of values selected from the range of 0.8 to 1.2.

The term “and/or” is used herein, in the description and in the claims, to refer to a single element alone or any combination of elements from the list in which the term and/or appears. In other words, a listing of two or more elements having the term “and/or” is intended to cover embodiments having any of the individual elements alone or having any combination of the listed elements. For example, the phrase “element A and/or element B” is intended to cover embodiments having element A alone, having element B alone, or having both elements A and B taken together. For example, the phrase “element A, element B, and/or element C” is intended to cover embodiments having element A alone, having element B alone, having element C alone, having elements A and B taken together, having elements A and C taken together, having elements B and C taken together, or having elements A, B, and C taken together.

In an embodiment, a composition or compound of the invention, such as an alloy or precursor to an alloy, is isolated or substantially purified. In an embodiment, an isolated or purified compound is at least partially isolated or substantially purified as would be understood in the art. In an embodiment, a substantially purified composition, compound or formulation of the invention has a chemical purity of 95%, optionally for some applications 99%, optionally for some applications 99.9%, optionally for some applications 99.99%, and optionally for some applications 99.999% pure.

### DETAILED DESCRIPTION OF THE INVENTION

In the following description, numerous specific details of the devices, device components and methods of the present invention are set forth in order to provide a thorough explanation of the precise nature of the invention. It will be apparent, however, to those of skill in the art that the invention can be practiced without these specific details.

Various aspects are contemplated herein, several of which are set forth in the paragraphs below. It is explicitly contemplated that any aspect or portion thereof can be combined to form an aspect. Moreover, for example, the term “any preceding aspect” means any aspect that appears prior to the aspect that contains such phrase is referenced (for example, the clause “Aspect 10: the method of any preceding aspect . . . ” means that any aspect prior to Aspect 10 is referenced, including Aspects 1-9). In addition, it is explicitly contemplated that any reference to Aspect X, where X is an integer corresponding to one of the below Aspects (e.g., Aspect 11), includes reference to Aspects AXa, AXb, and/or AXc, if present, etc. (e.g., Aspect 11a, Aspect 11b, Aspect 11c, and/or Aspect 11d).

Aspect 1: a method for treatment of a subject, the method comprising: topically administering a melanin formulation having an artificial melanin material (thereby administering artificial melanin material) to (or, to a site of) damaged skin of the subject; wherein the administered artificial melanin material comprises an extracellular artificial melanin material at (or, at the site of) the damaged skin; and facilitating skin healing at (or, in) the damaged skin via at least the extracellular artificial melanin material; wherein the step of facilitating skin healing comprises at least a portion of the extracellular artificial melanin material performing a therapeutic extracellular activity. The term “at the damaged skin” generally refers to a site of damaged skin or a region comprising at least a portion of the damaged skin, optionally but not necessarily also including some area/region immediately surrounding or adjacent to the damaged skin. The term “at the damaged skin” generally includes “on at least a portion of the damaged skin”. Facilitating skin healing generally, but not exclusively, refers to or includes accelerating or speeding-up skin healing compared to healing of a same or equivalent damaged skin without or in absence of treatment of the damaged skin with the melanin formulation, optionally in absence of any treatment. Optionally in this Aspect, at least 50% of the administered artificial melanin material is the extracellular artificial melanin material. Optionally in this Aspect, the extracellular artificial melanin material is present extracellularly as long as (or, for entirety of time of) being present at the damaged skin (e.g., until removed). Optionally in this Aspect, the method comprises at least a portion of the administered artificial melanin adsorbing, inactivating/deactivating, or otherwise transforming into a different specie one or more inflammatory factors, one or more enzymatic factors, and/or one or more apoptosis factors.

Aspect 2a: The method of Aspect 1, wherein the damaged skin is a closed wound. Aspect 2b: The method of Aspect 1, wherein the damaged skin is a closed wound having remnants of superficial skin attached.

Aspect 3a: The method of any preceding Aspect, wherein the damaged skin is associated with thermally-induced damage, chemically-induced damage, and/or radiation-induced damage. Aspect 3b: The method of any preceding Aspect, wherein the damaged skin is associated with thermally-induced damage, chemically-induced damage, radiation-induced damage (such as UV-induced damage), mechanical-friction damage, and/or infection cellulitis-induced damage.

Aspect 4a: The method of any preceding Aspect, wherein the damaged skin comprises one or more blisters. Aspect 4b: The method of any preceding Aspect, wherein the damaged skin comprises one or more visible blisters, one or more microscopic vesicles, separation of the epidermis from the dermis, or any combination of these.

Aspect 5: The method of any preceding Aspect, wherein at least a portion of the extracellular melanin material is in a stratum corneum at the damaged skin.

Aspect 6: The method of any preceding Aspect, wherein the damaged skin comprises extracellular free radical species; and wherein the therapeutic extracellular activity comprises the at least a portion of the extracellular artificial melanin material quenching at least the extracellular free radical species (or, at least a portion of the extracellular free radical species).

Aspect 7: The method of Aspect 6, wherein the extracellular free radical species comprise reactive oxygenated species.

Aspect 8: The method of Aspect 6 or 7, wherein at least a portion of the quenched extracellular free radical species are in a stratum corneum.

Aspect 9: The method of any preceding Aspect, wherein the damaged skin comprises inflammation; and wherein the step of facilitating skin healing comprises at least a portion of the administered artificial melanin material directly and/or indirectly reducing the inflammation.

Aspect 10: The method of Aspect 9, wherein the therapeutic extracellular activity comprises at least a portion of the extracellular artificial melanin material directly and/or indirectly reducing the inflammation.

Aspect 11a: The method of Aspect 9 or 10, wherein directly and/or indirectly reducing the inflammation comprises at least a portion of the administered artificial melanin adsorbing one or more inflammatory factors and/or one or more enzymatic factors. Aspect 11b: The method of Aspect 9 or 10, wherein directly and/or indirectly reducing the inflammation comprises at least a portion of the administered artificial melanin adsorbing, inactivating/deactivating, or otherwise transforming into a different specie one or more inflammatory factors, one or more enzymatic factors, and/or one or more apoptosis factors. Aspect 11c: The method of Aspect 9 or 10, wherein directly and/or indirectly reducing the inflammation comprises at least a portion of the administered artificial melanin directly and/or indirectly downregulating expression of one or more genes associates with inflammation. Aspect 11d: The method of Aspect 9 or 10, wherein directly and/or indirectly reducing the inflammation comprises at least a portion of the administered artificial melanin adsorbing, inactivating/deactivating, or otherwise transforming into a different specie one or more inflammatory factors, one or more enzymatic factors, and/or one or more apoptosis factors. Aspect 11e: The method of Aspect 9 or 10, wherein directly and/or indirectly reducing the inflammation comprises at least a portion of the administered artificial melanin directly and/or indirectly downregulating expression of Fas, Gzmb, Bcl2l1, Bcl2, Bax, or a combination of these.

Aspect 12a: The method of any of Aspects 9-11, wherein directly and/or indirectly reducing the inflammation comprises at least a portion of the extracellular artificial melanin adsorbing one or more extracellular inflammatory factors and/or one or more extracellular enzymatic factors. Aspect 12b: The method of any of Aspects 9-11, wherein directly and/or indirectly reducing the inflammation comprises at least a portion of the extracellular artificial melanin adsorbing one or more inflammatory factors, one or more enzymatic factors, and/or one or more apoptosis factors. Aspect 12c: The method of any of Aspects 9-11, wherein directly and/or indirectly reducing the inflammation comprises at least a portion of the extracellular artificial melanin adsorbing, inactivating/deactivating, or otherwise transforming into a different specie one or more inflammatory factors, one or more enzymatic factors, and/or one or more apoptosis factors.

Aspect 13a: The method of Aspect 11 or 12, wherein the one or more inflammatory factors and/or one or more enzymatic factors comprise TNFα, NOS, MMP9, one or more proteins associated with the MAPK/ERK pathway, and/or one or more enzymes associated with the MAPK/ERK pathway.

Aspect 14: The method of any of Aspects 11-13, wherein at least a portion of the adsorbed inflammatory factors and/or adsorbed extracellular enzymatic factors are in a stratum corneum.

Aspect 15a: The method of any preceding Aspect, wherein the step of facilitating comprises at least a portion of the administered artificial melanin directly and/or indirectly downregulating inflammation-related genes and/or apoptosis-related genes compared to when the artificial melanin materials is absent. Aspect 15b: The method of any preceding Aspect, wherein the step of facilitating comprises at least a portion of the extracellular artificial melanin directly and/or indirectly downregulating inflammation-related genes and/or apoptosis-related genes compared to when the artificial melanin materials is absent. Aspect 15c: The method of any preceding Aspect, wherein the step of facilitating comprises at least a portion of the extracellular artificial melanin directly and/or indirectly downregulating Fas, Gzmb, Bcl2l1, Bcl2, Bax, Cxcr3, Stat1, Stat3, Ccr2, Ece1, MMP9, or a combination of these. Generally, “when the artificial melanin materials is absent” refers to or corresponds to a comparative case of no melanin formulation being administered or an equivalent melanin formulation free of melanin being administered (e.g., to a same or equivalent skin damage suitable as a control), such as the control or vehicle cases discussed in Examples 1A-1B.

Aspect 16: The method of any preceding Aspect, wherein the step of facilitating comprises at least a portion of the administered artificial melanin directly and/or indirectly inhibiting apoptosis compared to when the artificial melanin materials is absent.

Aspect 17: The method of any preceding Aspect, wherein the step of facilitating skin healing further comprises at least a portion of the administered artificial melanin material performing a therapeutic intracellular activity.

Aspect 18: The method of Aspect 17, wherein the therapeutic intracellular activity comprises: quenching intracellular free radical species; and/or adsorbing one or more intracellular inflammatory factors and/or one or more intracellular enzymatic factors.

Aspect 19: The method of any preceding Aspect, wherein the skin healing comprises:
a reduction in a wound area comprising the damaged tissue;
a reduction of the inflammation at the damaged skin;
an increase in superoxide dismutase activity at the damaged skin;
a reduction in bi-fold skin thickness at the damaged skin;
a reduction in skin edema at the damaged skin;
a reduction in time to eschar detachment at the damaged skin;
a reduction in a depth of injury at the damaged skin; and/or
a stabilization of an epithelial layer at the damaged skin.

Aspect 20: The method of any preceding Aspect, wherein the skin healing is characterized by one or more of the following healing characteristics being less than the same one or more healing characteristics in absence of the artificial melanin material during the skin healing:
a total time (e.g., average total time) to reduce of an amount or concentration of a damaged tissue to 50% of that immediately prior to administering the melanin formulation;
a total time (e.g., average total time) to reduce inflammation at the damaged skin to 50% of that immediately prior to administering the melanin formulation;
a total time (e.g., average total time) to reduce bi-fold skin thickness at the damaged skin to 50% of that immediately prior to administering the melanin formulation;
a total time (e.g., average total time) to reduce skin edema at the damaged skin to 50% of that immediately prior to administering the melanin formulation;
a total time (e.g., average total time) to reduce a depth of injury at the damaged skin to 50% of that immediately prior to administering the melanin formulation; and/or
a total time (e.g., average total time) to eschar detachment in the region. Generally, “in absence of the artificial melanin material” refers to or corresponds to a comparative case of no melanin formulation being administered or an equivalent melanin formulation free of melanin being administered (e.g., to a same or equivalent skin damage suitable as a control).

Aspect 21: The method of any preceding Aspect, wherein the skin healing is characterized by one or more of the following healing characteristics being greater than the same one or more healing characteristics in absence of the artificial melanin material during the skin healing:
a rate (e.g., an average rate) of reduction in a wound area comprising the damaged skin;
a rate (e.g., an average rate) of reduction of inflammation at the damaged skin;
a rate (e.g., an average rate) of reduction of bi-fold skin thickness at the damaged skin;
a rate (e.g., an average rate) of reduction of skin edema at the damaged skin;
a rate (e.g., an average rate) of reduction of a depth of injury at the damaged skin;
a rate (e.g., an average rate) of stabilization of an epithelial layer at the damaged skin; and/or
an activity (e.g., an average activity) of superoxide dismutase at the damaged skin.

Aspect 22: The method of any preceding Aspect, wherein the artificial melanin material comprises porous artificial melanin material.

Aspect 23: The method of any preceding Aspect, wherein the artificial melanin material comprises artificial melanin particles.

Aspect 24: The method of any preceding Aspect, wherein the artificial melanin material comprises porous artificial melanin particles.

Aspect 25: The method of any preceding Aspect, wherein at least a portion of the artificial melanin material is characterized as eumelanin, pheomelanin, allomelanin, or a combination of these.

Aspect 26: The method of any preceding Aspect, wherein the artificial melanin material comprises amorphous artificial melanin material.

Aspect 27: The method of any preceding Aspect, wherein the artificial melanin material comprises a plurality of melanin oligomers and/or polymers; and wherein each melanin oligomer and/or polymer comprises a plurality of covalently-bonded melanin base units.

Aspect 28: The method of Aspect 27, wherein said melanin base units are one or more substituted or unsubstituted catechol-based monomer units, substituted or unsubstituted polyol-based monomer units, substituted or unsubstituted phenol-based monomer units, substituted or unsubstituted indole-based monomer units, substituted or unsubstituted benzothiazine-based monomer units, substituted or unsubstituted benzothiazole-based monomer units, substituted or unsubstituted dopamine-based monomer units, or any combination of these.

Aspect 29: The method of Aspect 27 or 28, wherein each of at least a portion of the artificial melanin material comprises allomelanin.

Aspect 30: The method of any of Aspects 27-29, wherein at least a portion of said melanin base units each independently comprises substituted or unsubstituted naphthalene.

Aspect 31: The method of any of Aspects 27-30, wherein at least a portion of said melanin base units each independently comprises dihydroxynaphthalene.

Aspect 32: The method of any of Aspects 27-31, wherein at least a portion of the artificial melanin material comprises melanin oligomers free of nitrogen.

Aspect 33: The method of any of Aspects 27-28, wherein at least a portion of the artificial melanin material comprises polydopamine.

Aspect 34: The method of claim any of Aspects 27, 28, or 33, wherein at least a portion of said melanin base units each independently comprises a substituted or unsubstituted dopamine monomer.

Aspect 35: The method of claim any of Aspects 27, 28, or 33-34, wherein at least a portion of said melanin base units each independently is selected from the group consisting of substituted or unsubstituted dihydroxydopamine monomer units, substituted or unsubstituted dioxydopamine monomer units, substituted or unsubstituted dihydroxynaphthalene monomer units, substituted or unsubstituted dihydroxyphenylalanine monomer units, substituted or unsubstituted dioxydopamine monomer units, substituted or unsubstituted tyrosine monomer units, substituted or unsubstituted tyramine monomer units, any derivative of these, and any combination of these.

Aspect 36: The method of claim any of Aspects 27, 28, or 33-35, wherein at least a portion of said melanin base units each independently is selected from the group consisting of 3,4-dihydroxydopamine monomer units, 3,4-dioxydopamine monomer units, 3,4-dihydroxynaphthalene monomer units, 1,8-dihydroxynapthalene, I-3,4-dihydroxyphenylalanine monomer units, and any combination of these.

Aspect 37: The method of any of Aspects 27-36, wherein at least 50% of the plurality of melanin oligomers are selected from the group consisting of monomer units, dimers, trimers, tetramers, pentamers, and any combination thereof.

Aspect 38: The method of any of Aspects 27-37, wherein each melanin oligomer is non-covalently associated with at least one other melanin oligomer or a melanin monomer via at least one of hydrogen bonding and π-π stacking of naphthalene rings; wherein the melanin monomer comprises the melanin base unit.

Aspect 39: The method of any of Aspects 27-38, wherein the artificial melanin material comprises a porous artificial melanin material (e.g., porous artificial melanin (nano)particles); and wherein the melanin oligomers and/or polymers of the porous artificial melanin material are arranged to form an internal structure having a plurality of pores; wherein the porous artificial melanin material is characterized by a pore volume per mass of material greater than or equal to 0.1 cm³/g and wherein at least a portion of said pores have at least one size dimension greater than or equal to 0.5 nm.

Aspect 40: The method of any of Aspects 27-39, wherein the artificial melanin material comprises artificial melanin particles; and wherein at least a portion of the artificial melanin particles are solid particles, hollow particles, lacey particles, or any combinations of these.

Aspect 41: The method of any preceding Aspect, wherein at least a portion of the artificial melanin material comprises one or more selenomelanin polymers; wherein the one or more selenomelanin polymers comprise a plurality of covalently bonded selenomelanin base units; and wherein a chemical formula of each of the one or more selenomelanin base units comprises at least one selenium atom.

Aspect 42: The method of Aspect 41, wherein each selenomelanin polymer is a pheomelanin.

Aspect 43: The method of Aspect 41 or 42, wherein the chemical formula of each of the one or more selenomelanin base units comprises at least one covalent bond with each of the at least one selenium atom.

Aspect 44: The method of any of Aspects 41-43, wherein the chemical formula of each of the one or more selenomelanin base units comprises a substituted or unsubstituted benzoselenazine or a derivative thereof, a substituted or unsubstituted benzoselenazole or a derivative thereof, a substituted or unsubstituted 7,10-dihydro-2H-[1,4]selenazino[3,2-h]isoquinolin-3(4H)-one or a derivative thereof, a substituted or unsubstituted benzoselenazinone or a derivative thereof, or any combination of these.

Aspect 45a: The method of any preceding Aspect, wherein the artificial melanin material comprises artificial melanin particles having a size (or, characteristic size, such as diameter) selected from the range of 10 nm to 1000 nm (wherein any intermediate range is explicitly contemplated), optionally greater than 10 nm to less than 1000 nm, optionally 10 nm to 50 nm, optionally 20 nm to 1000 nm, optionally 100 nm to 1000 nm, optionally 150 nm to 1000 nm, optionally 200 nm to 1000 nm, optionally 220 nm to 1000 nm, optionally 240 nm to 1000 nm, optionally 250 nm to 1000 nm, optionally 275 nm to 1000 nm, optionally 300 nm to 1000 nm, optionally 240 nm to 900 nm. Aspect 45b: The method of any preceding Aspect, wherein the artificial melanin material comprises artificial melanin particles having an average size (or, average characteristic size, such as average diameter) selected from the range of 10 nm to 1000 nm (wherein any intermediate range is explicitly contemplated), optionally greater than 10 nm to less than 1000 nm, optionally 10 nm to 50 nm, optionally 20 nm to 1000 nm, optionally 100 nm to 1000 nm, optionally 150 nm to 1000 nm, optionally 200 nm to 1000 nm, optionally 220 nm to 1000 nm, optionally 240 nm to 1000 nm, optionally 250 nm to 1000 nm, optionally 275 nm to 1000 nm, optionally 300 nm to 1000 nm, optionally 240 nm to 900 nm, optionally 300 nm to 900 nm, optionally 250 nm to 800 nm, optionally 250 nm to 800 nm, optionally 275 nm to 800 nm. Aspect 45c: The method of any preceding Aspect, wherein the artificial melanin material comprises artificial melanin particles having a peak (or, maximum) size (or, characteristic peak size, such as peak diameter) selected from the range of 10 nm to 1000 nm (wherein any intermediate range is explicitly contemplated), optionally greater than 10 nm to less than 1000 nm, optionally 10 nm to 50 nm, optionally 20 nm to 1000 nm, optionally 100 nm to 1000 nm, optionally 150 nm to 1000 nm, optionally 200 nm to 1000 nm, optionally 220 nm to 1000 nm, optionally 240 nm to 1000 nm, optionally 250 nm to 1000 nm, optionally 275 nm to 1000 nm, optionally 300 nm to 1000 nm, optionally 240 nm to 900 nm, optionally 300 nm to 900 nm, optionally 250 nm to 800 nm, optionally 250 nm to 800 nm, optionally 275 nm to 800 nm.

Aspect 46a: The method of any preceding Aspect, wherein a concentration of the artificial melanin material in the melanin formulation is selected from the range of 0.5 mg/mL to 100 mg/mL, optionally 1 mg/mL to 100 mg/mL, optionally 2 mg/mL to 100 mg/mL, optionally 5 mg/mL to 100 mg/mL, optionally 10 mg/mL to 100 mg/mL, optionally 15 mg/mL to 100 mg/mL, optionally 20 mg/mL to 100 mg/mL, optionally 50 mg/mL to 100 mg/mL, optionally 0.5 mg/mL to 50 mg/mL, optionally 1 mg/mL to 50 mg/mL, optionally 2 mg/mL to 50 mg/mL, optionally 5 mg/mL to 50 mg/mL, optionally 10 mg/mL to 100 mg/mL, optionally 20 mg/mL to 50 mg/mL, optionally 0.5 mg/mL to 20 mg/mL, optionally 0.5 mg/mL to 10 mg/mL. Aspect 46b: The method of any preceding Aspect, wherein a concentration of the artificial melanin material in the melanin formulation is selected from the range of 0.1 mg/mL to 1000 mg/mL, wherein any intermediate range is explicitly contemplated.

Aspect 47a: The method of any preceding Aspect, wherein the melanin formulation comprises artificial melanin particles having a concentration selected from the range of 0.5 mg/mL to 100 mg/mL, optionally 1 mg/mL to 100 mg/mL, optionally 2 mg/mL to 100 mg/mL, optionally 5 mg/mL to 100 mg/mL, optionally 10 mg/mL to 100 mg/mL, optionally 15 mg/mL to 100 mg/mL, optionally 20 mg/mL to 100 mg/mL, optionally 50 mg/mL to 100 mg/mL, optionally 0.5 mg/mL to 50 mg/mL, optionally 1 mg/mL to 50 mg/mL, optionally 2 mg/mL to 50 mg/mL, optionally 5 mg/mL to 50 mg/mL, optionally 10 mg/mL to 100 mg/mL, optionally 20 mg/mL to 50 mg/mL, optionally 0.5 mg/mL to 20 mg/mL, optionally 0.5 mg/mL to 10 mg/mL. Aspect 47b: The method of any preceding Aspect, wherein the melanin formulation comprises artificial melanin particles having a concentration selected from the range of 0.1 mg/mL to 1000 mg/mL, wherein any intermediate range is explicitly contemplated.

Aspect 48: The method of any preceding Aspect, wherein the melanin formulation is such that the step of administering comprises forming a layer of the artificial melanin material over at least a portion of the damaged skin.

Aspect 49: The method of any preceding Aspect, wherein the melanin formulation is hydrophilic and/or wherein the artificial melanin material is hydrophilic.

Aspect 50: The method of any preceding Aspect, wherein the melanin formulation comprises one or more additives.

Aspect 51: The method of any preceding Aspect, wherein the melanin formulation is characterized by being a cream or ointment.

Aspect 52: The method of any preceding Aspect, wherein the melanin formulation comprises a hydrogel.

Aspect 53: The method of any preceding Aspect, wherein the melanin formulation is free of artificial melanin material loaded or functionalized with a non-melanin therapeutic agent.

Aspect 54: The method of any preceding Aspect, wherein the melanin formulation is free of hollow and/or semi-hollow melanin particles carrying a non-melanin therapeutic agent.

Aspect 55: The method of any preceding Aspect, wherein the melanin formulation is free of a non-melanin therapeutic agent.

Aspect 56: The method of any preceding Aspect, wherein the step of administering occurs after a skin damage event has occurred in the region of the subject.

Aspect 57: The method of Aspect 56 comprising damaging skin to form the damaged skin prior to the step of administering.

Aspect 58: The method of any preceding Aspect, comprising repeating the step of administering.

Aspect 59: The method of any preceding Aspect, wherein the subject is a mammal and/or the healed skin is mammalian skin.

Aspect 60: The method of any preceding Aspect, wherein at least 50% (optionally at least 60%, optionally at least 70%, optionally at least 80%, optionally at least 90%) of the administered artificial melanin material is the extracellular artificial melanin material (present extracellularly at the damaged skin during step of facilitating skin healing).

Aspect 61: The method of any preceding Aspect, wherein the extracellular artificial melanin material is present extracellularly as long as (or, for entirety of time of) being present at the damaged skin (e.g., until removed).

Various potentially useful descriptions, background information, applications/uses of embodiments herein, terminology (to the extent not inconsistent with the terms as defined herein), mechanisms, compositions, methods, definitions, and/or other embodiments may optionally be found in: International Patent App. No. PCT/US2017/041596 (published as International Pat. Pub. No. WO2018013609A2), International Patent App. No. PCT/US2020/039769 (published as International Pat. Pub. No. WO2021021350A3), International Patent App. No. PCT/US2020/057902 (published as International Pat. Pub. No. WO2021087076A1), and International Patent App. No. PCT/US2020/057939 (published as International Pat. Pub. No. WO2021096692A1), each of which is incorporated herein by reference to the extent not inconsistent herewith.

Optionally in some embodiments, such as some of Aspects 1-61, the artificial melanin material comprises synthetic melanin particles, also referred to herein as artificial melanin particles, also referred to interchangeably herein as artificial melanin-like particles or synthetic melanin-like particles, prepared by spontaneous oxidation of melanin monomers in an aqueous solution under alkaline conditions, to produce biocompatible, synthetic analogues of naturally occurring melanosomes.

Optionally in some embodiments, such as some of Aspects 1-61, such as some of Aspects 1-61, the artificial melanin material comprises synthetic melanin particles comprising non-natural particles composed of (e.g. comprising, consisting of, or consisting essentially of) melanin that is not bound to, conjugated to, attached to, coated by, encompassed by or otherwise associated with a lipid (i.e. a lipid comprising one or more proteins such as the lipid (plasma) membrane of a melanocyte or melanosome). Optionally in some embodiments, such as some of Aspects 1-61, the artificial melanin material comprises synthetic melanin particles comprising non-natural particles composed of (e.g. consisting of or consisting essentially of) melanin that is not bound to, conjugated to, attached to, coated by, encompassed by or otherwise associated with a proteinaceous lipid (i.e. a lipid comprising one or more proteins such as the lipid (plasma) membrane of a melanocyte or melanosome).

Optionally in some embodiments, such as some of Aspects 1-61, the artificial melanin material comprises synthetic melanin particles comprising melanin polymer being a fused ring melanin polymer which includes (e.g. consists of or consists essentially of) monomers of fused ring heteroaryl monomer and/or fused ring heterocycloalkyl monomers. Optionally in some embodiments, such as some of Aspects 1-61, the artificial melanin material comprises synthetic melanin particles comprising melanin polymer being a fused ring metal-binding melanin polymer comprising a melanin polymer bound to a plurality of transitions metals including but not limited to iron. Optionally in some embodiments, such as some of Aspects 1-61, the artificial melanin material comprises synthetic melanin particles comprising a fused ring melanin polymer being a dopamine monomer, including but not limited to dihydoxydopamine, 3,4-dihydoxydopamine, dioxydpoamine and/or 3,4-dioxydopamine. Optionally in some embodiments, such as some of Aspects 1-61, each of a fused ring heteroaryl monomer and/or fused ring heterocycloalkyl monomer may be substituted with one or more substituents selected from hydroxyl, carboxyl and/or oxy. Optionally in some embodiments, such as some of Aspects 1-61, each of the fused ring heteroaryl monomer is a 6,6-fused ring heteroaryl monomer, a 5,6-fused ring heteroaryl monomer or 6,5-fused ring heteroaryl monomer and each of the fused ring heterocycloalkyl moieties is a 6,6-fused ring heterocycloalkyl monomer, a 5,6-fused ring heterocycloalkyl 1 monomer or 6,5-fused ring heterocycloalkyl monomer. Optionally in some embodiments, such as some of Aspects 1-61, the fused ring heteroaryl monomers and/or fused ring heterocycloalkyl monomers (in the monovalent or bivalent form) are selected from indole (such as dihydroxyindole, 5,6-dihydroxyindole (DHI), 5,6-dihydroxyindole-2-carboxylic acid, dioxyindole, 5,6-dioxyindole, 5,6-droxyindole-2-carboxylic acid), benzothiazine, benzothiazole. Optionally in some embodiments, such as some of Aspects 1-61, the fused ring monomeric units of the fused ring melanin polymer are dihydroxy fused ring units (e.g. dihydroxy fused ring heteroaryl monomers and/or dihydroxy fused ring heterocycloalkyl monomers) wherein the hydroxy substituents are attached to adjacent carbons of a 6 membered ring (e.g. 6 membered carbon ring) of a fused ring monomer (also referred to herein as a “catechol fused ring monomer”). Optionally in some embodiments, such as some of Aspects 1-61, the fused ring melanin polymer may also contained oxidized versions of the dihydroxy fused ring units wherein one or both of the hydroxyl substituents are oxy substituents. Optionally in some embodiments, such as some of Aspects 1-61, the synthetic melanin particles can be in the form of a sphere, hollow sphere, nanorod, worm-like configuration, cylindrical configuration, and the like, with at least one dimensional axis thereof of from about 1 nm to about 1000 nm, from about 1 nm to about 1000 nm, from about 50 nm to about 500 nm, or from about 100 nm to about 300 nm, preferably with a high aspect ratio. Optionally in some embodiments, such as some of Aspects 1-61, synthetic melanin particles is in the form of a sphere of from about 50 nm to about 500 nm, from about 100 nm to about 300 nm, from about 150 nm to about 250 nm, or about 250 nm in average diameter. Optionally in some embodiments, such as some of Aspects 1-61, the synthetic melanin particles are in the form of a hollow sphere, optionally filled with silica. Optionally in some embodiments, such as some of Aspects 1-61, the synthetic melanin particles are capable of functioning as a pigment. Optionally in some embodiments, such as some of Aspects 1-61, the synthetic melanin particles are synthetic melanin nanoparticles.

Optionally in some embodiments, such as some of Aspects 1-61, an artificial melanin material disclosed herein comprises artificial melanin nanoparticles, wherein: each melanin nanoparticle of the plurality of artificial melanin nanoparticles comprises a plurality of melanin oligomers; each melanin oligomer comprises a plurality of covalently-bonded melanin base units; and each melanin base unit comprises substituted or unsubstituted naphthalene.

Optionally in some embodiments, such as some of Aspects 1-61, an artificial melanin material disclosed herein comprises artificial melanin nanoparticles, wherein: each melanin nanoparticle of the plurality of artificial melanin nanoparticles comprises a plurality of melanin oligomers; each melanin oligomer comprises a plurality of covalently-bonded melanin base units; and the plurality of artificial melanin nanoparticles are characterized by a peak size selected from the range of 100 nm to 300 nm and a polydispersity index selected to be less than or equal to 0.10, and optionally for some embodiments a polydispersity index selected to be less than or equal to 0.3 and optionally for some embodiments a polydispersity index selected to be less than or equal to 0.2. Optionally, the plurality of artificial melanin nanoparticles are characterized by a peak size selected from the range of 100 nm to 200 nm and a polydispersity index selected to be less than or equal to 0.10.

Optionally in some embodiments, such as some of Aspects 1-61, an artificial melanin material disclosed herein comprises artificial melanin nanoparticles, wherein: each melanin nanoparticle of the plurality of artificial melanin nanoparticles comprises a plurality of melanin oligomers; each melanin oligomer comprises a plurality of covalently-bonded melanin base units; and the plurality of artificial melanin nanoparticles exhibits structural color. Optionally, the plurality of artificial melanin nanoparticles exhibits structural color when the plurality of artificial melanin nanoparticles are in the form of a layer or film, such as a monolayer or thicker, or in the form of a pellet, such as a free-standing pellet, for example. Optionally, the plurality of artificial melanin nanoparticles exhibits structural color when the plurality of artificial melanin nanoparticles are in the form of a packed and/or ordered structure. Optionally, the plurality of artificial melanin nanoparticles exhibits structural color when the plurality of artificial melanin nanoparticles are dried or otherwise deposited onto a substrate.

Optionally in some embodiments, such as some of Aspects 1-61, an artificial melanin material disclosed herein comprises artificial melanin nanoparticles wherein: each melanin nanoparticle of the plurality of artificial melanin nanoparticles comprises a plurality of melanin oligomers; each melanin oligomer comprises a plurality of covalently-bonded melanin base units; and at least 50% of the plurality of melanin oligomers are selected from the group consisting of monomers, dimers, trimers, tetramers, pentamers, and any combination thereof. The monomers, dimers, trimers, tetramers, and pentamers have one, two, three, four, and five melanin base units, respectively. Optionally, at least 30%, optionally at least 40%, optionally at least 50%, optionally at least 60%, optionally at least 80%, of the plurality of melanin oligomers are selected from the group consisting of dimers, trimers, tetramers, pentamers, and any combination thereof, and the artificial melanin nanoparticles further comprise monomers. Optionally, at least 50% of the plurality of melanin oligomers are selected from the group consisting of dimers, trimers, tetramers, pentamers, and any combination thereof, and the artificial melanin nanoparticles further comprise monomers. Optionally, at least 30%, optionally at least 40%, optionally at least 50%, optionally at least 60%, optionally at least 80%, of the plurality of melanin oligomers are selected from the group consisting of dimers, trimers, tetramers, and any combination thereof, and the artificial melanin nanoparticles further comprise monomers. Optionally, at least 50% of the plurality of melanin oligomers are selected from the group consisting of dimers, trimers, tetramers, and any combination thereof, and the artificial melanin nanoparticles further comprise monomers. Optionally, at least 30% by mass, optionally at least 40% by mass, optionally at least 50% by mass, optionally at least 60% by mass, optionally at least 80% by mass, of each or of each of at least 80% of the plurality of artificial melanin nanoparticles is the monomers (each monomer having only one melanin base unit) and/or the melanin oligomers selected from the group consisting of dimers, trimers, tetramers, pentamers and any combination thereof. Optionally, at least 30% by mass, optionally at least 40% by mass, optionally at least 50% by mass, optionally at least 60% by mass, optionally at least 80% by mass, of each or of each of at least 80% of the plurality of artificial melanin nanoparticles is the monomers (each monomer having only one melanin base unit) and the melanin oligomers selected from the group consisting of dimers, trimers, tetramers, pentamers and any combination thereof. Optionally, at least 30% by mass, optionally at least 40% by mass, optionally at least 50% by mass, optionally at least 60% by mass, optionally at least 80% by mass, of each or of each of at least 80% of the plurality of artificial melanin nanoparticles is the monomers (each monomer having only one melanin base unit) and/or the melanin oligomers selected from the group consisting of dimers, trimers, tetramers, and any combination thereof. Optionally, at least 30% by mass, optionally at least 40% by mass, optionally at least 50% by mass, optionally at least 60% by mass, optionally at least 80% by mass, of each or of each of at least 80% of the plurality of artificial melanin nanoparticles is the monomers (each monomer having only one melanin base unit) and the melanin oligomers selected from the group consisting of dimers, trimers, tetramers, and any combination thereof.

Optionally in some embodiments, such as some of Aspects 1-61, an artificial melanin material disclosed herein comprises artificial melanin nanoparticles, wherein: each melanin nanoparticle of the plurality of artificial melanin nanoparticles comprises a plurality of melanin oligomers; each melanin oligomer comprises a plurality of covalently-bonded melanin base units; and each nanoparticle has a sphericity of less than 0.90 and has a shape characterized as at least one of: walnut-like, a collapsed sphere or collapsed ellipsoid, and a sphere or ellipsoid having a plurality of indentations.

Optionally in some embodiments, such as some of Aspects 1-61, an artificial melanin material disclosed herein comprises artificial melanin nanoparticles, wherein: each melanin nanoparticle of the plurality of artificial melanin nanoparticles comprises a plurality of melanin oligomers; each melanin oligomer comprises a plurality of covalently-bonded melanin base units; and the plurality of artificial melanin nanoparticles are characterized by a radical scavenging activity greater than that of polydopamine nanoparticles having the same diameter as the plurality of artificial melanin nanoparticles under otherwise identical condition. Optionally, the plurality of artificial melanin nanoparticles are characterized by a radical scavenging activity at least 5%, optionally at least 10%, optionally at least 15%, optionally at least 20%, greater than that of polydopamine nanoparticles having the same diameter as the plurality of artificial melanin nanoparticles under otherwise identical condition.

Optionally in some embodiments, such as some of Aspects 1-61, an artificial melanin material disclosed herein comprises artificial melanin nanoparticles, wherein: each melanin base unit comprises substituted or unsubstituted naphthalene. Optionally in some embodiments, such as some of Aspects 1-61, an artificial melanin material disclosed herein comprises artificial melanin nanoparticles, wherein: each melanin base unit comprises dihydroxynaphthalene. Optionally in some embodiments, such as some of Aspects 1-61, an artificial melanin material disclosed herein comprises artificial melanin nanoparticles, wherein: each melanin base unit comprises 1,8-dihydroxynaphthalene. According to certain embodiments, each melanin base unit comprises a structure having the formula FX1:

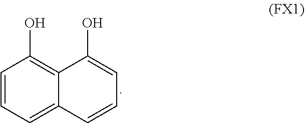 [SMILES: Oc1cccc2cccc(O)c12]

Optionally in some embodiments, such as some of Aspects 1-61, an artificial melanin material disclosed herein comprises artificial melanin nanoparticles, wherein: each melanin oligomer is free of nitrogen. According to certain embodiments, at least 20%, optionally at least 40%, optionally at least 50%, optionally at least 80% of the plurality of melanin oligomers are dimers having two covalently-bonded melanin base units. Optionally in some embodiments, such as some of Aspects 1-61, an artificial melanin material disclosed herein comprises artificial melanin nanoparticles, wherein: 20% to 80% of the plurality of melanin oligomers are dimers having two covalently-bonded melanin base units. Optionally in some embodiments, such as some of Aspects 1-61, an artificial melanin material disclosed herein comprises artificial melanin nanoparticles, wherein: at least 50% of the plurality of melanin oligomers are selected from the group consisting of monomers, dimers, trimers, tetramers, pentamers, and any combination thereof. The monomers, dimers, trimers, tetramers, and pentamers have one, two, three, four, and five melanin base units, respectively. Optionally, at least 30%, optionally at least 40%, optionally at least 50%, optionally at least 60%, optionally at least 80%, of the plurality of melanin oligomers are selected from the group consisting of dimers, trimers, tetramers, pentamers, and any combination thereof, and the artificial melanin nanoparticles further comprise monomers. Optionally in some embodiments, such as some of Aspects 1-61, an artificial melanin material disclosed herein comprises artificial melanin nanoparticles, wherein: at least 40% of the plurality of melanin oligomers are selected from the group consisting of monomers, dimers, trimers, tetramers, pentamers, and any combination thereof. Optionally in some embodiments, such as some of Aspects 1-61, an artificial melanin material disclosed herein comprises artificial melanin nanoparticles, wherein: at least 20%, optionally at least 40%, optionally at least 80%, of the plurality of melanin oligomers are selected from the group consisting of monomers, dimers, and trimers, and any combination thereof. Optionally in some embodiments, such as some of Aspects 1-61, an artificial melanin material disclosed herein comprises artificial melanin nanoparticles, wherein: at least 50% of the plurality of melanin oligomers are selected from the group consisting of monomers, dimers, and trimers, and any combination thereof. Optionally in some embodiments, such as some of Aspects 1-61, an artificial melanin material disclosed herein comprises artificial melanin nanoparticles, wherein: at least 30% by mass, optionally at least 40% by mass, optionally at least 50% by mass, optionally at least 60% by mass, optionally at least 80% by mass, of each or of each of at least 80% of the plurality of artificial melanin nanoparticles is the monomers (each monomer having only one melanin base unit) and/or the melanin oligomers selected from the group consisting of dimers, trimers, tetramers, pentamers and any combination thereof. Optionally in some embodiments, such as some of Aspects 1-61, an artificial melanin material disclosed herein comprises artificial melanin nanoparticles, wherein: at least 30% by mass, optionally at least 40% by mass, optionally at least 50% by mass, optionally at least 60% by mass, optionally at least 80% by mass, of each or of each of at least 80% of the plurality of artificial melanin nanoparticles is the monomers (each monomer having only one melanin base unit) and the melanin oligomers selected from the group consisting of dimers, trimers, tetramers, pentamers and any combination thereof. Optionally in some embodiments, such as some of Aspects 1-61, an artificial melanin material disclosed herein comprises artificial melanin nanoparticles, wherein: at least 30% by mass, optionally at least 40% by mass, optionally at least 50% by mass, optionally at least 60% by mass, optionally at least 80% by mass, of each or of each of at least 80% of the plurality of artificial melanin nanoparticles is the monomers (each monomer having only one melanin base unit) and/or the melanin oligomers selected from the group consisting of dimers, trimers, tetramers, and any combination thereof. Optionally in some embodiments, such as some of Aspects 1-61, an artificial melanin material disclosed herein comprises artificial melanin nanoparticles, wherein: at least 30% by mass, optionally at least 40% by mass, optionally at least 50% by mass, optionally at least 60% by mass, optionally at least 80% by mass, of each or of each of at least 80% of the plurality of artificial melanin nanoparticles is the monomers (each monomer having only one melanin base unit) and the melanin oligomers selected from the group consisting of dimers, trimers, tetramers, and any combination thereof. Optionally in some embodiments, such as some of Aspects 1-61, an artificial melanin material disclosed herein comprises artificial melanin nanoparticles, wherein: each melanin oligomer is non-covalently associated with at least one other melanin oligomer via at least one of hydrogen bonding and rr-rr stacking of naphthalene rings. Optionally in some embodiments, such as some of Aspects 1-61, an artificial melanin material disclosed herein comprises artificial melanin nanoparticles, wherein: each melanin oligomer is non-covalently associated with at least one other melanin oligomer or melanin monomer via at least one of hydrogen bonding and rr-rr stacking of naphthalene rings. Optionally in some embodiments, such as some of Aspects 1-61, an artificial melanin material disclosed herein comprises artificial melanin nanoparticles, wherein: a melanin monomer comprises the melanin base unit.

Optionally in some embodiments, such as some of Aspects 1-61, an artificial melanin material disclosed herein comprises artificial melanin nanoparticles, wherein: at least 50%, optionally at least 75%, optionally at least 90%, optionally at least 95%, of the plurality of nanoparticles is characterized by a sphericity of greater than 0.90. Optionally in some embodiments, such as some of Aspects 1-61, an artificial melanin material disclosed herein comprises artificial melanin nanoparticles, wherein: the plurality nanoparticles is characterized by a polydispersity index less than or equal to 0.10. Optionally in some embodiments, such as some of Aspects 1-61, an artificial melanin material disclosed herein comprises artificial melanin nanoparticles, wherein: each nanoparticle has a size characteristics, such as diameter, selected from the range of 10 nm to less than or equal to 1000 nm, optionally 100±50 nm to 300±50 nm. Optionally in some embodiments, such as some of Aspects 1-61, an artificial melanin material disclosed herein comprises artificial melanin nanoparticles, wherein: an average size characteristics, such as average diameter, of the artificial melanin nanoparticles is selected from the range of 10 nm to less than or equal to 1000 nm, optionally 20 nm to 500 nm, optionally 100 nm to 900 nm, optionally 200 nm to 900 nm, optionally 100 nm to 800 nm, optionally greater than 250 nm and less than 1000 nm. Optionally in some embodiments, such as some of Aspects 1-61, an artificial melanin material disclosed herein comprises artificial melanin nanoparticles, wherein: each of at least 55% (optionally at least 75%, optionally at least 80%, optionally at least 85%) of the nanoparticles has a size characteristic, such as diameter, selected from the range of greater than 200 nm, optionally greater than 250 nm, to less than 1000 nm. Optionally in some embodiments, such as some of Aspects 1-61, an artificial melanin material disclosed herein comprises artificial melanin nanoparticles, wherein: each nanoparticle has a size characteristics, such as diameter, selected from the range of 10 nm to less than or equal to 1000 nm, optionally 100 nm to 300 nm. Optionally in some embodiments, such as some of Aspects 1-61, an artificial melanin material disclosed herein comprises artificial melanin nanoparticles, wherein: each nanoparticle has a size characteristics, such as diameter, selected from the range of 20 nm to 300±50 nm. Optionally in some embodiments, such as some of Aspects 1-61, an artificial melanin material disclosed herein comprises artificial melanin nanoparticles, wherein: the plurality of artificial melanin nanoparticles are characterized by a peak size selected from the range of 10 nm to less than or equal to 1000 nm, optionally 100 nm to 300 nm. Optionally in some embodiments, such as some of Aspects 1-61, an artificial melanin material disclosed herein comprises artificial melanin nanoparticles, wherein: the plurality of artificial melanin nanoparticles are characterized by a peak size selected from the range of 10 nm to less than or equal to 1000 nm, optionally 100 nm to 200 nm. Optionally in some embodiments, such as some of Aspects 1-61, an artificial melanin material disclosed herein comprises artificial melanin nanoparticles, wherein: the plurality of artificial melanin nanoparticles are characterized by a peak size selected from the range of 50 nm to 300 nm, optionally 50 nm to 200 nm.

Optionally in some embodiments, such as some of Aspects 1-61, an artificial melanin material in the melanin formulation disclosed herein comprises artificial melanin nanoparticles, wherein the melanin formulation comprises a solvent or solvent mixture being at least 50% water, optionally at least 75% water, optionally at least 90% water, optionally at least 95%, by volume. According to certain embodiments, the solvent or solvent mixture comprises an organic solvent. According to certain embodiments, the solvent or solvent mixture comprises a buffer. According to certain embodiments, the organic solvent comprises methanol, ethanol, acetonitrile, acetone dichloromethane, dimethylformamide, ethyl acetate, acetone, or any combination thereof. In some embodiments, such as some of Aspects 1-61, artificial melanin nanoparticles are allowed to further age or further oxidize after synthesis. In some embodiments, such as some of Aspects 1-61, aging or further oxidation of the nanoparticles affects the solubility or dispersibility (in the melanin formulation), such as increasing stability in the presence of organic solvents. According to certain embodiments, the nanoparticles in the melanin formulation are characterized by a zeta potential or an average zeta potential selected from the range of −50 mV to −10 mV, optionally −40 to −20 mV, optionally in a solvent or solvent solution that is at least 95% water by volume. According to certain embodiments, the nanoparticles in the melanin formulation are stably dispersed without forming precipitates after at least 5 hours at a concentration selected from the range of 0.01 mg/mL to 5 mg/mL, optionally 0.01 mg/mL to 1 mg/mL, optionally within 20% of 0.1 mg/mL. According to certain embodiments, the nanoparticles in the melanin formulation are stably dispersed without forming precipitates after at least 12 hours at a concentration selected from the range of 0.01 mg/mL to 5 mg/mL, optionally 0.01 mg/mL to 1 mg/mL, optionally within 20% of 0.1 mg/mL.

Optionally in some embodiments, such as some of Aspects 1-61, an artificial melanin material disclosed herein comprises melanin monomers each melanin monomer having substituted or unsubstituted naphthalene. Optionally in some embodiments, such as some of Aspects 1-61, an artificial melanin material disclosed herein comprises melanin monomers each melanin monomer having dihydroxynaphthalene. Optionally in some embodiments, such as some of Aspects 1-61, an artificial melanin material disclosed herein comprises melanin monomers each melanin monomer having 1,8-dihydroxynaphthalene. Optionally in some embodiments, such as some of Aspects 1-61, an artificial melanin material disclosed herein comprises melanin monomers each melanin monomer being free of nitrogen. According to certain embodiments, the artificial melanin material is not derived or extracted from a biological source or a living organism.

Optionally in some embodiments, such as some of Aspects 1-61, an artificial melanin material disclosed herein or plurality of artificial melanin nanoparticles thereof is characterized by a radical scavenging activity greater than that of polydopamine nanoparticles having the same diameter as the plurality of artificial melanin nanoparticles under otherwise identical condition. Optionally in some embodiments, such as some of Aspects 1-61, an artificial melanin material disclosed herein or plurality of artificial melanin nanoparticles thereof is characterized by a radical scavenging activity at least 10%, optionally at least 15%, optionally at least 50%, greater than that of polydopamine nanoparticles having the same diameter as the plurality of artificial melanin nanoparticles under otherwise identical condition. Optionally in some embodiments, such as some of Aspects 1-61, an artificial melanin material disclosed herein or plurality of artificial melanin nanoparticles thereof is characterized by a radical scavenging activity of at least 0.012 mol/g using an assay of 2,2-diphenyl-1-(2,4,6-trinitrophenyl) hydrazyl (DPPH).

Optionally in some embodiments, such as some of Aspects 1-61, an artificial melanin material disclosed herein comprises one or more porous artificial melanin materials. Optionally in some embodiments, such as some of Aspects 1-61, an artificial melanin material disclosed herein comprises an porous artificial melanin material comprising: (i) one or more melanin oligomers, polymers or a combination thereof; wherein the one or more melanin oligomers and/or polymers comprise a plurality of covalently-bonded melanin base units; wherein the melanin oligomers and/or polymers are arranged to form an internal structure having a plurality of pores. Optionally in some embodiments, such as some of Aspects 1-61, an artificial melanin material disclosed herein comprises an porous artificial melanin material comprising: (i) one or more melanin oligomers, polymers or a combination thereof; wherein the one or more melanin oligomers and/or polymers comprise a plurality of covalently-bonded melanin base units; wherein the melanin oligomers and/or polymers are arranged to form an internal structure having a plurality of pores; wherein the porous artificial melanin material is characterized by a pore volume per mass of material greater than or equal to 0.1 cm³/g, optionally greater than or equal to 0.3 cm³/g, and wherein at least a portion of the pores have at least one size dimension, such as cross section dimension or longitudinal dimension, greater than or equal to 0.5 nm. Optionally in some embodiments, such as some of Aspects 1-61, an artificial melanin material disclosed herein comprises a porous artificial melanin material characterized by an average pore volume per mass of material selected from the range of 0.1 cm³/g to 0.6 cm³/g, and optionally 0.1 to 1 cm³/g and optionally 0.3 cm³/g to 0.6 cm³/g. Optionally in some embodiments, such as some of Aspects 1-61, an artificial melanin material disclosed herein comprises a porous artificial melanin material being a microporous material or a mesoporous material. Optionally in some embodiments, such as some of Aspects 1-61, an artificial melanin material disclosed herein comprises a porous artificial melanin material wherein the pores of the porous artificial melanin material include micropores each having at least one average size dimension, such as a cross sectional dimension and/or longitudinal dimension, selected from the range of 0.5 nm to 2.5 nm, and optionally 0.5 nm to 1.3 nm. Optionally in some embodiments, such as some of Aspects 1-61, an artificial melanin material disclosed herein comprises a porous artificial melanin material wherein the pores of the porous artificial melanin material include mesopores each having at least one average size dimension, such as a cross sectional dimension and/or longitudinal dimension, selected from the range of 2 nm to 50 nm, and optionally 2 nm to 25 nm. Optionally in some embodiments, such as some of Aspects 1-61, an artificial melanin material disclosed herein comprises a porous artificial melanin material wherein the pores are characterized by a distribution of pore sizes over the range of 0.5 nm to 50 nm. Optionally in some embodiments, such as some of Aspects 1-61, an artificial melanin material disclosed herein comprises a porous artificial melanin material wherein the pores of the internal structure are formed by organization of the melanin oligomers and/or polymers of the porous artificial melanin material. Optionally in some embodiments, such as some of Aspects 1-61, an artificial melanin material disclosed herein comprises a porous artificial melanin material wherein the pores of the internal structure are formed by close packing and/or self-assembly of the melanin oligomers and/or polymers of the porous artificial melanin material. Optionally in some embodiments, such as some of Aspects 1-61, an artificial melanin material disclosed herein comprises a porous artificial melanin material wherein the pores of the internal structure are formed by templating of the melanin oligomers and/or polymers of the porous artificial melanin material. Optionally in some embodiments, such as some of Aspects 1-61, an artificial melanin material disclosed herein comprises a porous artificial melanin material wherein the pores are not uniformly distributed throughout the porous melanin materials, for example, because the material is non-crystalline and/or amorphous. Optionally in some embodiments, such as some of Aspects 1-61, an artificial melanin material disclosed herein comprises a porous artificial melanin material wherein the porous artificial melanin material is an at least partially non-crystalline material and/or an amorphous material. Optionally in some embodiments, such as some of Aspects 1-61, an artificial melanin material disclosed herein comprises a porous artificial melanin material wherein the pores of the internal structure are randomly distributed. Optionally in some embodiments, such as some of Aspects 1-61, an artificial melanin material disclosed herein comprises a porous artificial melanin material wherein the pores of the internal structure are provided in repeating structures the amorphous porous artificial melanin material provided in an at least partial non-crystalline or amorphous state. Optionally in some embodiments, such as some of Aspects 1-61, an artificial melanin material disclosed herein comprises a porous artificial melanin material wherein the pores of porous artificial melanin material include one or more pore types selected from the group of cylindrical pores, channel-like pores, slit-shape pores, ink-bottle pores and any combination of these.

Optionally in some embodiments, such as some of Aspects 1-61, an artificial melanin material disclosed herein comprises a porous artificial melanin material having porous melanin particles, such as nanoparticles. Optionally in some embodiments, such as some of Aspects 1-61, an artificial melanin material disclosed herein comprises a porous artificial melanin material having porous melanin particles characterized by an average size selected from the range of 20 nm to 500 nm in diameter. Optionally in some embodiments, such as some of Aspects 1-61, an artificial melanin material disclosed herein comprises a porous artificial melanin material having porous melanin particles being one or more of solid particles, hollow particles, lacey particles, and any combinations of these. Optionally in some embodiments, such as some of Aspects 1-61, an artificial melanin material disclosed herein comprises a porous artificial melanin material having solid porous artificial melanin particles, for example, with pores distributed throughout the particle, for example uniformly distributed or randomly distributed, and without a hollow configuration. Optionally in some embodiments, such as some of Aspects 1-61, an artificial melanin material disclosed herein comprises a porous artificial melanin material having lacey porous artificial melanin particles, for example, with pores distributed throughout the particle, for example uniformly distributed or randomly distributed, and without a hollow configuration. Optionally in some embodiments, such as some of Aspects 1-61, an artificial melanin material disclosed herein comprises a porous artificial melanin material having hollow porous artificial melanin particles.

Optionally in some embodiments, such as some of Aspects 1-61, an artificial melanin material disclosed herein comprises a porous artificial melanin material having porous melanin particles that are purified or isolated.

Optionally in some embodiments, such as some of Aspects 1-61, an artificial melanin material disclosed herein comprises a porous artificial melanin material having melanin base units that are one or more substituted or unsubstituted catechol-based monomers, substituted or unsubstituted polyol-based monomers, substituted or unsubstituted phenol-based monomers, substituted or unsubstituted indole-based monomers, substituted or unsubstituted benzothiazine-based monomers, substituted or unsubstituted benzothiazole-based monomers, substituted or unsubstituted dopamine-based monomers or any combination of these.

Optionally in some embodiments, such as some of Aspects 1-61, an artificial melanin material disclosed herein comprises a porous artificial melanin material having or being allomelanin. In some embodiments, such as some of Aspects 1-61, for example, at least a portion of, and optionally all of, the melanin base units each independently comprises substituted or unsubstituted naphthalene. In some embodiments, such as some of Aspects 1-61, for example, at least a portion of, and optionally all of, the melanin base units each independently comprises dihydroxynaphthalene. In some embodiments, such as some of Aspects 1-61, for example, at least a portion of, and optionally all of, the melanin base units each independently comprises 1,8-dihydroxynaphthalene. In some embodiments, such as some of Aspects 1-61, for example, at least a portion of, and optionally all of, the melanin base units each independently comprises a structure having the formula FX1:

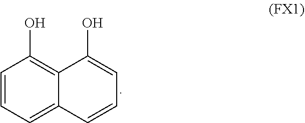 [SMILES: Oc1cccc2cccc(O)c12]

In some embodiments, such as some of Aspects 1-61, for example, each melanin oligomer is free of nitrogen.

Optionally in some embodiments, such as some of Aspects 1-61, an artificial melanin material disclosed herein comprises a porous artificial melanin material having polydopamine. In some embodiments, such as some of Aspects 1-61, for example, at least a portion of, and optionally all of, the melanin base units each independently comprises a substituted or unsubstituted dopamine monomer. In some embodiments, such as some of Aspects 1-61, for example, at least a portion of, and optionally all of, the melanin base units each independently are selected from the group consisting of substituted or unsubstituted dihydroxydopamine monomers, substituted or unsubstituted dioxydopamine monomers, substituted or unsubstituted dihydroxynaphthalene monomers, substituted or unsubstituted dioxydopamine monomers and any combination of these. In some embodiments, such as some of Aspects 1-61, for example, at least a portion of, and optionally all of, the melanin base units each independently are selected from the group consisting of 3,4-dihydroxydopamine monomers, 3,4-dioxydopamine monomers, 3,4-dihydroxynaphthalene monomers, and any combination of these.

Optionally in some embodiments, such as some of Aspects 1-61, an artificial melanin material disclosed herein comprises a porous artificial melanin material having allomelanin.

Optionally in some embodiments, such as some of Aspects 1-61, an artificial melanin material disclosed herein comprises substituted or unsubstituted catechol-based or polyol-based compounds. Optionally in some embodiments, such as some of Aspects 1-61, an artificial melanin material disclosed herein comprises substituted or unsubstituted dopamine monomers. Optionally in some embodiments, such as some of Aspects 1-61, an artificial melanin material disclosed herein comprises substituted or unsubstituted: dopamine monomers, 1,8-Dihydroxynaphthalene or its derivative, tyrosine monomers, tyramine monomers, amino acids, phenolamines, catecholamines, or any combination of these. Optionally in some embodiments, such as some of Aspects 1-61, an artificial melanin material disclosed herein comprises substituted or unsubstituted: dopamine monomers, tyrosine monomers, tyramine monomers, or a combination of these. Optionally in some embodiments, such as some of Aspects 1-61, an artificial melanin material disclosed herein is free of phenol derivatives, resorcinol, and/or paraphenylenediamine. Optionally, the dopamine monomers are selected from the group consisting of substituted or unsubstituted: dihydoxydopamine monomers, dihydoxydopamine dimers, dihydoxydopamine oligomers, dioxydopamine monomers, dioxydopamine dimers, dioxydopamine oligomers, dihydroxynapthalene monomers, dihydroxynapthalene dimers, dihydroxynapthalene oligomers, dioxydopamine monomers, dioxydopamine dimers, dioxydopamine oligomers, and any combination of these. Optionally, the dopamine monomers are selected from the group consisting of tyrosine and derivatives, phenol and derivatives, resorcinol and derivatives, and any combinations thereof. Optionally, the dopamine monomers are selected from the group consisting of phenol, resorcinol, L-DOPA, tyrosine and any combinations thereof. Optionally, the dopamine monomers are selected from the group consisting of cysteine derivatives, chalcogenides derivatives, selenocysteine, and any combinations thereof. Optionally in some embodiments, such as some of Aspects 1-61, an artificial melanin material disclosed herein comprises one or more monomers selected from the group consisting of:

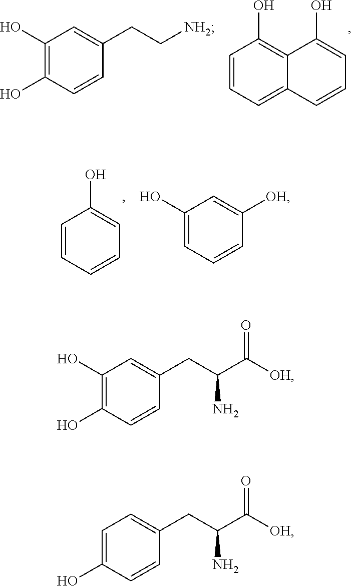

any combinations thereof, and any derivatives thereof. Optionally in some embodiments, such as some of Aspects 1-61, an artificial melanin material disclosed herein comprises one or more monomers having the formula (FX2):

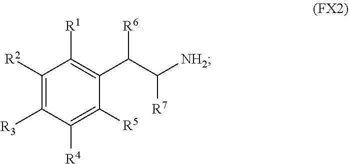

wherein one or more (optionally one, optionally two) of R¹-R⁷ is —OH and wherein each of the other of R¹-R⁷ is a functional group. Optionally, the each of the other of R¹-R⁷ is selected from the group consisting of hydrogen, C₁-C₁₀ alkyl, C₃-C₁₀ cycloalkyl, C₅-C₁₀ aryl, C₅-C₁₀ heteroaryl, C₁-C₁₀ acyl, C₁-C₁₀ hydroxyl, C₁-C₁₀ alkoxy, C₂-C₁₀ alkenyl, C₂-C₁₀ alkynyl, C₅-C₁₀ alkylaryl, —CO₂R³⁰, —CONR³¹R³², —COR³³, —NR³⁹R⁴⁰, —NR⁴¹COR⁴², C₁-C₁₀ alkyl halide, acrylate, or catechol; wherein each of R³⁰-R⁴² is independently hydrogen, C₁-C₁₀ alkyl or C₅-C₁₀ aryl. Optionally, for any method disclosed herein, the artificial melanin precursors are one or more monomers having the formula (FX3):

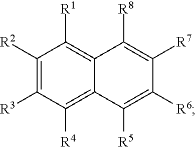

wherein one or more (optionally one, optionally two) of R¹-R⁸ is —OH and wherein each of the other of R¹-R⁸ is a functional group. Optionally, the each of the other of R¹-R⁷ is selected from the group consisting of hydrogen, C₁-C₁₀ alkyl, C₃-C₁₀ cycloalkyl, C₅-C₁₀ aryl, C₅-C₁₀ heteroaryl, C₁-C₁₀ acyl, C₁-C₁₀ hydroxyl, C₁-C₁₀ alkoxy, C₂-C₁₀ alkenyl, C₂-C₁₀ alkynyl, C₅-C₁₀ alkylaryl, —CO₂R³⁰, —CONR³¹R³², —COR³³, —NR³⁹R⁴⁰, —NR⁴¹COR⁴², C₁-C₁₀ alkyl halide, acrylate, or catechol; wherein each of R³⁰-R⁴² is independently hydrogen, C₁-C₁₀ alkyl or C₅-C₁₀ aryl. Optionally in some embodiments, such as some of Aspects 1-61, an artificial melanin material disclosed herein comprises one or more thiol-reactive moieties. Optionally, the thiol-reactive moieties are one or more groups selected from the group consisting of a thiol, maleimide, pyridyl disulfide-based compound, alkene, alkyl halide and any combinations thereof. Optionally in some embodiments, such as some of Aspects 1-61, an artificial melanin material disclosed herein comprises one or more monomers having the formula (FX2) or (FX3), wherein one or more of R¹-R⁸ is a thiol-reactive moiety, such as a thiol, maleimide, pyridyl disulfide-based compound, alkene, alkyl halide and any combinations thereof.

Optionally in some embodiments, such as some of Aspects 1-61, an artificial melanin material disclosed herein comprises one or more artificial selenomelanin materials having: one or more selenomelanin polymers; wherein the one or more selenomelanin polymers comprise a plurality of covalently bonded selenomelanin base units; and wherein a chemical formula of each of the one or more selenomelanin base units comprises at least one selenium atom. Optionally, each selenomelanin polymer is a pheomelanin. Optionally, each of the selenomelanin monomers is an amino acid. Optionally, the chemical formula of each of the one or more selenomelanin base units comprises at least one covalent bond with each of the at least one selenium atom. Optionally, each of the one or more selenomelanin polymers is not bound to, conjugated to, attached to, coated by, encompassed by, or otherwise chemically associated with a natural or biological proteinaceous matrix, component, or lipid. Optionally, each of the plurality of selenomelanin base units is not bound to, conjugated to, attached to, coated by, encompassed by, or otherwise chemically associated with a natural or biological proteinaceous matrix, component, or lipid. Optionally, the chemical formula of each of the one or more selenomelanin base units comprises one selenium atom and two covalent bonds with the selenium atom. Optionally, the chemical formula of each of the one or more selenomelanin base units comprises a substituted or unsubstituted benzoselenazine or a derivative thereof, a substituted or unsubstituted benzoselenazole or a derivative thereof, a substituted or unsubstituted 7,10-dihydro-2H-[1,4]selenazino[3,2-h]isoquinolin-3(4H)-one or a derivative thereof, a substituted or unsubstituted benzoselenazinone or a derivative thereof, or any combination of these. Optionally, each of the one or more selenomelanin base units comprises a moiety characterized by formula FX11, FX12, FX13A, FX13B, FX14, a combination of any of these, or a derivative of any of these:

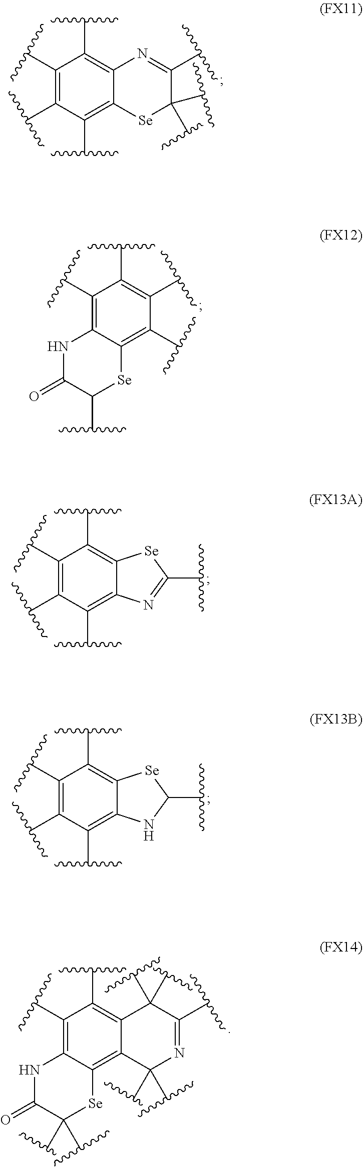 [SMILES: *C1=Nc2c(*)c(*)c(*)c(*)c2[Se]C1(*)* ; *c1c(*)c(*)c2c(c1*)NC(=O)C(*)[Se]2 ; *c1nc2c(*)c(*)c(*)c(*)c2[se]1 ; *c1c(*)c(*)c2c(c1*)NC(*)[Se]2 ; *C1=NC(*)(*)c2c3c(c(*)c(*)c2C1(*)*)NC(=O)C(*)(*)[Se]3]

Optionally, each of the one or more selenomelanin base units comprises a moiety characterized by formula FX11, FX12, FX13A, FX13B, FX14, or a combination of any of these. Optionally, each of the one or more selenomelanin base units comprises a moiety characterized by formula FX11, FX12, FX13A, FX13B, FX14, or a combination of any of these. Optionally, each of the one or more selenomelanin base units comprises a moiety characterized by formula FX11, FX12, FX13A, FX13B, or FX14. Optionally, each of the one or more selenomelanin base units comprises a moiety characterized by formula FX11. Optionally, an artificial selenomelanin material is one or a plurality of artificial selenomelanin nanoparticles, artificial selenomelanin layers, or artificial selenomelanin thin films. Optionally, an artificial selenomelanin material is one or a plurality of artificial selenomelanin nanoparticles. Optionally, each of the one or more selenomelanin base units comprises a heterocyclic moiety comprising a Se as a member of its ring structure. Optionally, each of the one or more selenomelanin base units comprises a heterocyclic moiety comprising a Se and a N as members of its ring structure. Optionally, each of the one or more selenomelanin base units comprises a moiety characterized by formula FX23, FX24, FX25, FX26, FX27, a derivative of any one of these, or a combination of any of these:

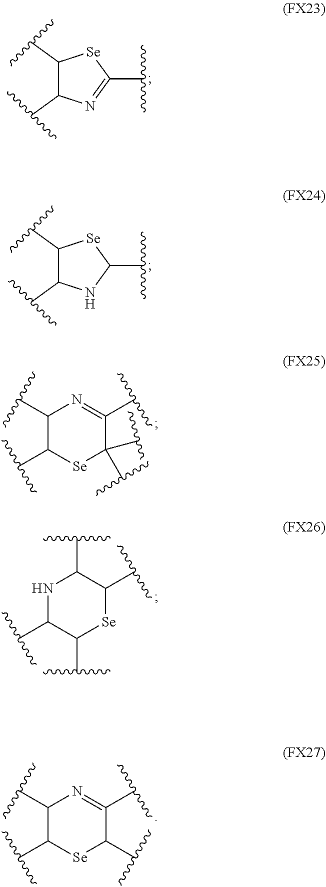 [SMILES: *C1=NC(*)C(*)[Se]1 ; *C1NC(*)C(*)[Se]1 ; *C1=NC(*)C(*)[Se]C1(*)* ; *C1NC(*)C(*)[Se]C1* ; *C1=NC(*)C(*)[Se]C1*]

Optionally, each of the one or more selenomelanin base units comprises a moiety characterized by formula FX23, FX24, FX25, FX26, FX27, or a combination of any of these. Optionally, each of the one or more selenomelanin base units comprises a moiety characterized by formula FX23, FX24, FX25, FX26, or FX27. Optionally, each of the selenomelanin monomers is characterized by formula FX15 FX16, FX17, FX18, FX19 FX20, or FX21:

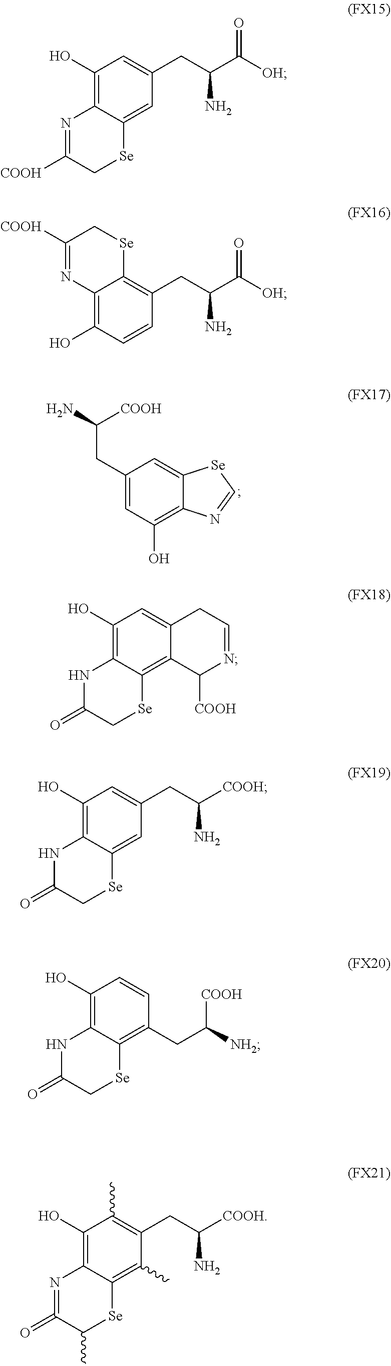

Optionally in some embodiments, such as some of Aspects 1-61, an artificial melanin material disclosed herein comprises one or more artificial selenomelanin materials wherein each of the one or more selenomelanin polymers is not bound to, conjugated to, attached to, coated by, encompassed by, or otherwise chemically associated with a natural or biological proteinaceous matrix, component, or lipid.

Optionally in some embodiments, such as some of Aspects 1-61, an artificial melanin material disclosed herein comprises one or more artificial selenomelanin materials wherein the chemical formula of each of the one or more selenomelanin base units comprises benzoselenazine and wherein the material comprises benzoselenazine at a concentration selected from the range of 10 wt. % to 100 wt. %. Optionally in some embodiments, such as some of Aspects 1-61, an artificial melanin material disclosed herein comprises one or more artificial selenomelanin materials having benzoselenazine at a concentration selected from the range of 50 wt. % to 60 wt. %. For example, the chemical formula of each of the one or more selenomelanin base units comprises benzoselenazine and the material can comprise benzoselenazine at a concentration of 55 wt. %. Optionally in some embodiments, such as some of Aspects 1-61, an artificial melanin material disclosed herein comprises one or more artificial selenomelanin materials characterized a concentration of selenium selected from the range of 2 wt. % to 23 wt. %. For example, the artificial selenomelanin material can be characterized a concentration of selenium of 12 wt. %.

Optionally in some embodiments, such as some of Aspects 1-61, an artificial melanin material in a melanin formulation disclosed herein comprises one or more artificial selenomelanin materials wherein the solvent or solvent mixture is at least 50% water. Optionally in some embodiments, such as some of Aspects 1-61, an artificial melanin material in a melanin formulation disclosed herein comprises one or more artificial selenomelanin materials having artificial selenomelanin nanoparticles characterized by an absolute value of a Zeta potential selected from the range of 15 mV to 50 mV, preferably 20 mV to 50 mV, optionally 15 mV to 40 mV, optionally 20 mV to 40 mV, optionally 15 mV to 30 mV, optionally 20 mV to 30 mV, optionally 17 mV to 34 mV. (The absolute value, or modulus, of a real number is the non-negative value of the real number without regard to its sign.) Optionally, the sign of the Zeta potential corresponding to the artificial selenomelanin nanoparticles in the artificial selenomelanin nanoparticle dispersion is negative. Optionally in some embodiments, such as some of Aspects 1-61, an artificial melanin material in a melanin formulation disclosed herein comprises one or more artificial selenomelanin materials having artificial selenomelanin nanoparticles being size-stable at nanoparticle concentrations selected from the range of 0.1 mg/mL to 104 mg/mL with respect to an average size of the nanoparticle at a concentration of 0.1 mg/mL. Optionally in some embodiments, such as some of Aspects 1-61, an artificial melanin material in a melanin formulation disclosed herein comprises one or more artificial selenomelanin materials having artificial selenomelanin nanoparticles being size-stable in the dispersion having a pH of 11, preferably at least 11, with respect to an average size of the nanoparticle in the dispersion having a pH of 7. Optionally in some embodiments, such as some of Aspects 1-61, an artificial melanin material in a melanin formulation disclosed herein comprises one or more artificial selenomelanin materials having artificial selenomelanin nanoparticles being size-stable when exposed to a concentration of NaCl selected from the range of 50 mM to 250 mM, preferably a concentration of NaCl being 250 mM, in the dispersion, with respect to an average size of the nanoparticles in an equivalent dispersion free of NaCl. Optionally in some embodiments, such as some of Aspects 1-61, an artificial melanin material in a melanin formulation disclosed herein comprises one or more artificial selenomelanin materials having artificial selenomelanin nanoparticles being stably dispersed in the dispersion for at least 7 days, preferably at least 14 days, preferably at least 60 days under ambient conditions.

Optionally in some embodiments, such as some of Aspects 1-61, an artificial melanin material in a melanin formulation disclosed herein comprises one or more artificial selenomelanin materials having artificial selenomelanin nanoparticles being characterized by a melanin purity of at least 20%, optionally at least 25%, optionally at least 30%, preferably at least 50%, more preferably at least 70%, further more preferably at least 80%, yet further more preferably at least 90%, more preferably for some applications at least 95%, still more preferably for some applications at least 99%, still further more preferably for some application at least 99.9%. Optionally in some embodiments, such as some of Aspects 1-61, an artificial melanin material in a melanin formulation disclosed herein comprises one or more artificial selenomelanin materials having artificial selenomelanin nanoparticles wherein each of at least 50%, optionally at least 75%, preferably at least 90%, more preferably at least 95%, further more preferably at least 99%, of the plurality of artificial melanin nanoparticles comprises a selenomelanin polymer having selenomelanin base units comprising a moiety characterized by formula FX11, FX12, FX13A, FX13B, FX14, or a combination of any of these:

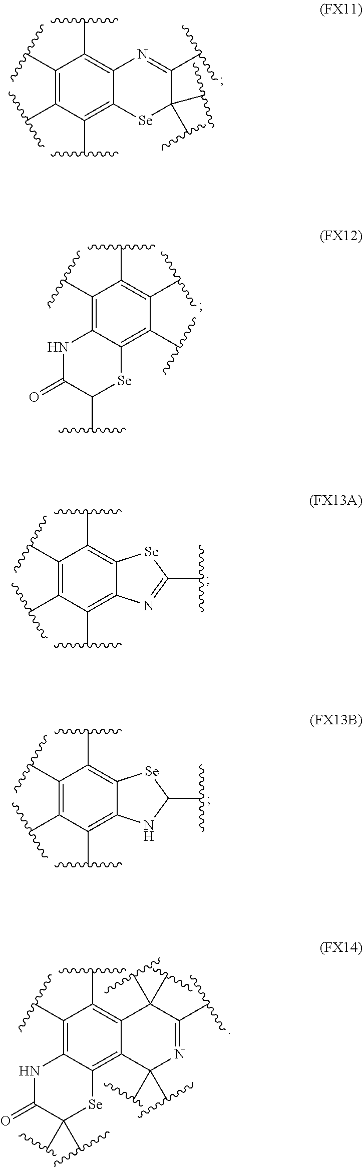 [SMILES: *C1=Nc2c(*)c(*)c(*)c(*)c2[Se]C1(*)* ; *c1c(*)c(*)c2c(c1*)NC(=O)C(*)[Se]2 ; *c1nc2c(*)c(*)c(*)c(*)c2[se]1 ; *c1c(*)c(*)c2c(c1*)NC(*)[Se]2 ; *C1=NC(*)(*)c2c3c(c(*)c(*)c2C1(*)*)NC(=O)C(*)(*)[Se]3]

Optionally in some embodiments, such as some of Aspects 1-61, an artificial melanin material in a melanin formulation disclosed herein comprises one or more artificial selenomelanin materials having artificial selenomelanin nanoparticles wherein each of at least 50%, optionally at least 75%, preferably at least 90%, more preferably at least 95%, further more preferably at least 99%, of the plurality of artificial melanin nanoparticles comprises a selenomelanin polymer having selenomelanin base units comprises a heterocyclic moiety comprising a Se as a member of its ring structure. Optionally in some embodiments, such as some of Aspects 1-61, an artificial melanin material in a melanin formulation disclosed herein comprises one or more artificial selenomelanin materials having artificial selenomelanin nanoparticles wherein each of at least 50%, optionally at least 75%, preferably at least 90%, more preferably at least 95%, further more preferably at least 99%, of the plurality of artificial melanin nanoparticles comprises a selenomelanin polymer having selenomelanin base units comprises a heterocyclic moiety comprising a Se and a N as members of its ring structure. Optionally in some embodiments, such as some of Aspects 1-61, an artificial melanin material in a melanin formulation disclosed herein comprises one or more artificial selenomelanin materials having artificial selenomelanin nanoparticles wherein each of at least 50%, optionally at least 75%, preferably at least 90%, more preferably at least 95%, further more preferably at least 99%, of the plurality of artificial melanin nanoparticles comprises a selenomelanin polymer having selenomelanin base units comprises a moiety characterized by formula FX23, FX24, FX25, FX26, FX27, a derivative of any one of these, or a combination of any of these. Optionally in some embodiments, such as some of Aspects 1-61, an artificial melanin material in a melanin formulation disclosed herein comprises one or more artificial selenomelanin materials having artificial selenomelanin nanoparticles wherein each of at least 50%, optionally at least 75%, preferably at least 90%, more preferably at least 95%, further more preferably at least 99%, of the plurality of artificial melanin nanoparticles comprises a selenomelanin polymer having selenomelanin base units comprises a moiety characterized by formula FX23, FX24, FX25, FX26, FX27, or a combination of any of these. Optionally in some embodiments, such as some of Aspects 1-61, an artificial melanin material in a melanin formulation disclosed herein comprises one or more artificial selenomelanin materials having artificial selenomelanin nanoparticles wherein each of at least 50%, optionally at least 75%, preferably at least 90%, more preferably at least 95%, further more preferably at least 99%, of the plurality of artificial melanin nanoparticles comprises a selenomelanin polymer having selenomelanin base units comprises a moiety characterized by formula FX23, FX24, FX25, FX26, or FX27. Optionally in some embodiments, such as some of Aspects 1-61, an artificial melanin material in a melanin formulation disclosed herein comprises one or more artificial selenomelanin materials having artificial selenomelanin nanoparticles wherein each of the one or more selenomelanin nanoparticles is not bound to, conjugated to, attached to, coated by, encompassed by, or otherwise chemically associated with a natural or biological proteinaceous matrix, component, or lipid. Optionally in some embodiments, such as some of Aspects 1-61, an artificial melanin material in a melanin formulation disclosed herein comprises one or more artificial selenomelanin materials having artificial selenomelanin nanoparticles wherein each of at least 50%, optionally at least 75%, preferably at least 80%, preferably at least 90%, more preferably at least 95%, further more preferably at least 99%, of the artificial selenomelanin nanoparticles is free of artificial melanin monomers. Optionally in some embodiments, such as some of Aspects 1-61, an artificial melanin material in a melanin formulation disclosed herein comprises one or more artificial selenomelanin materials having artificial selenomelanin nanoparticles wherein each of the artificial selenomelanin nanoparticles is free of artificial melanin monomers. For example, artificial selenomelanin materials, such as nanoparticles, are extensively washed with HCl solution (e.g., once) and pure water (e.g., 3 times), as a result of which the artificial selenomelanin materials, or dispersion or formulations thereof, can be free of artificial selenomelanin monomers, as characterized by solid-state NMR, UV-Vis spectra, etc. Optionally, a melanin formulation disclosed herein comprises a concentration of melanin monomers being less than IC50 of the monomers, respectively. Optionally in some embodiments, such as some of Aspects 1-61, an artificial melanin material in a melanin formulation disclosed herein comprises one or more artificial selenomelanin materials having artificial selenomelanin nanoparticles wherein each of the artificial selenomelanin nanoparticles is external (extracellular) of a biological cell.

The invention can be further understood by the following non-limiting examples.

### Example 1A: Synthetic Melanin Promotes Tissue Repair

This Example comprises some exemplary but not limiting methods, materials, processes, techniques, compositions, formulations, etc., useful in the practice of the invention as well as exemplary but not limiting data, discussion, and hypotheses, without wishing to be bound by any particular theory. It is recognized that regardless of the ultimate correctness of any mechanistic explanation or hypothesis, an embodiment of the invention can nonetheless be operative and useful.

Melanin is a pervasive biopolymer widely dispersed across different organisms.¹ In humans, melanin is produced by melanosome organelles, which are found in melanocytes. Uniquely, melanocytes are the only known cells that can excrete organelles extracellularly, primarily to keratinocytes in the skin.² Melanin is most notably known as a dark brown or black pigment in the skin and functions as a broadband radiation adsorbent.^{3, 4} Melanosome production increases in response to increased UV exposure aiding in UV radiation protection. Properties of melanin are not limited photoprotection; it has a myriad of other functions uncharacteristic to those of biological pigments that can account for its ubiquitous presence in nature including structural coloration,⁵ metal chelation,⁶ small molecule absorption,⁷⁻⁹ and thermoregulation.¹⁰ These diverse functions can be attributed to melanin's chemical nature and morphology. Melanin exhibits many intermolecular interactions such as hydrogen-bonding, pi-pi stacking, and covalent interactions.⁸ The abundance of intermolecular interactions is due to melanin's various functional groups, including catechols, carboxyls, and amines. Due to the different oxidation states of its catechol groups, melanin can accept and donate electrons allowing for redox activity.¹¹ Furthermore, it has antioxidant activity because of its electron donation capabilities. Melanin has been shown to quench radical oxygen species (ROS) and other reactive oxygenated species that are mutagenic, carcinogenic, cause production of DNA strand breaks, and create DNA-protein crosslinks.^{1, 4, 12} The ability to prevent cell damage via ROS pathways may be the primary way melanin protects against radiation. The use of antioxidants for wound healing has been studied and it is contemplated that they are able to accelerate wound healing through mediating oxidative stress.^{13, 14} These properties suggest melanin's role in organisms and in our own skin may be more complex than we know.

In this Example, we synthesize well defined nanoscale Synthetic Melanin Particles (SMP). SMP is synthesized through the oxidative polymerization of dopamine. Synthetic melanin mimics have been used extensively in biomedical applications including bioimaging¹⁵, drug delivery¹⁶, and theranostics¹⁷ due to their biocompatibility and myriad of functionalities. Of interest is melanin's ability to scavenge radical oxygen species for the purpose of wound healing. As a consequence of thermal, chemical, or puncture wounds, different pathways can be signaled to release extra ROS having adverse wound healing effects.^{18, 19} In this Example, we employ low and high surface area Synthetic Melanin Particles, synthesized based on previous methods.²⁰ Low and high surface area particles are applied to investigate the importance of melanin availability to the skin after chemical and thermal wound models. With the application of SMP, there is a decrease in the inflammation and wound area. There is an observed advantage of the high surface SMP over the low surface SMP in tissue repair. The mechanism behind these wound healing properties is contemplated and an increase in superoxide dismutase activity is found.

### Exemplary Results:

### PDA Nanoparticles: Synthesis and Characterization:

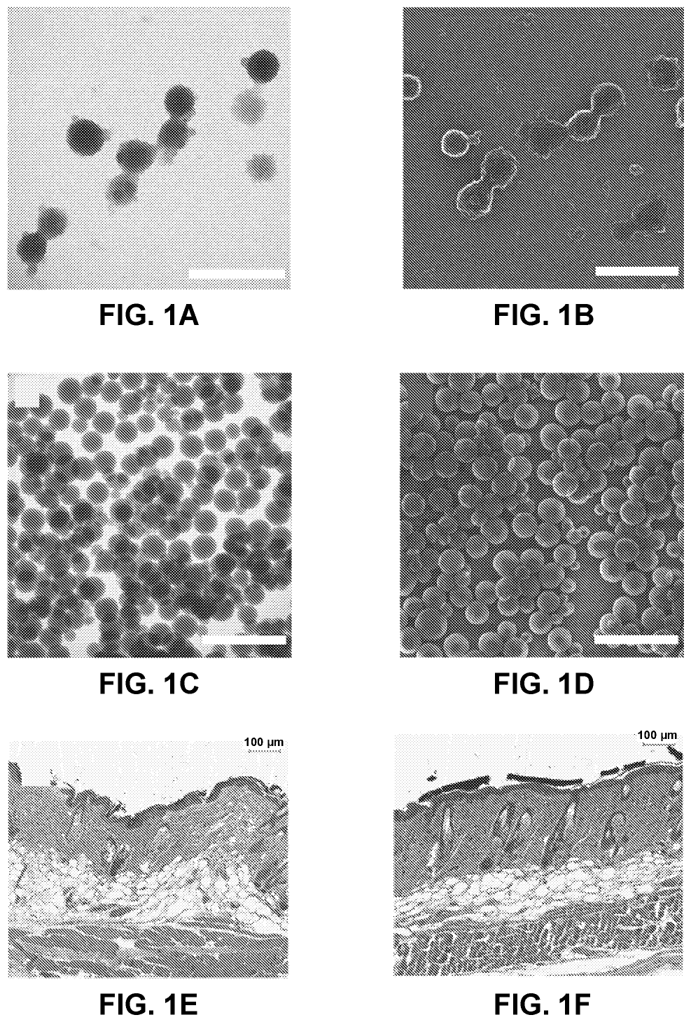

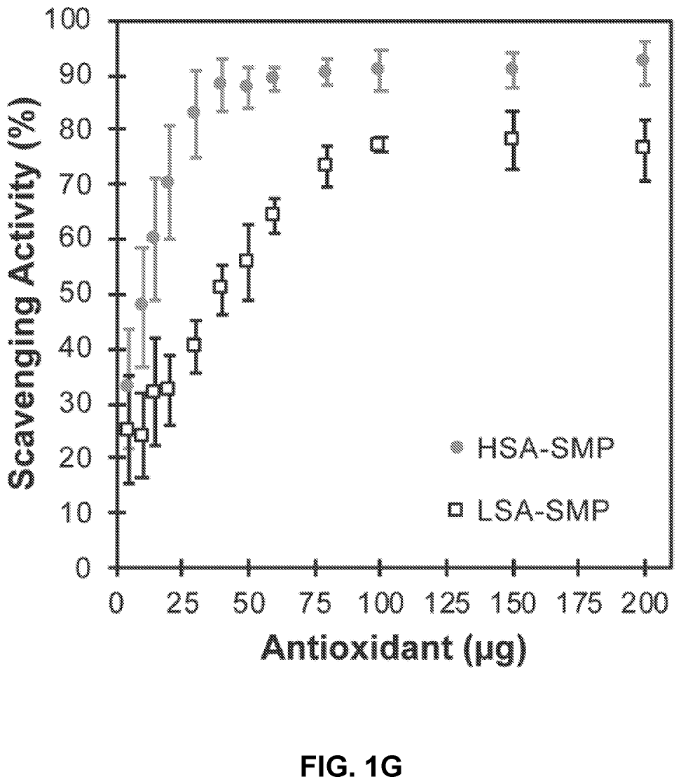

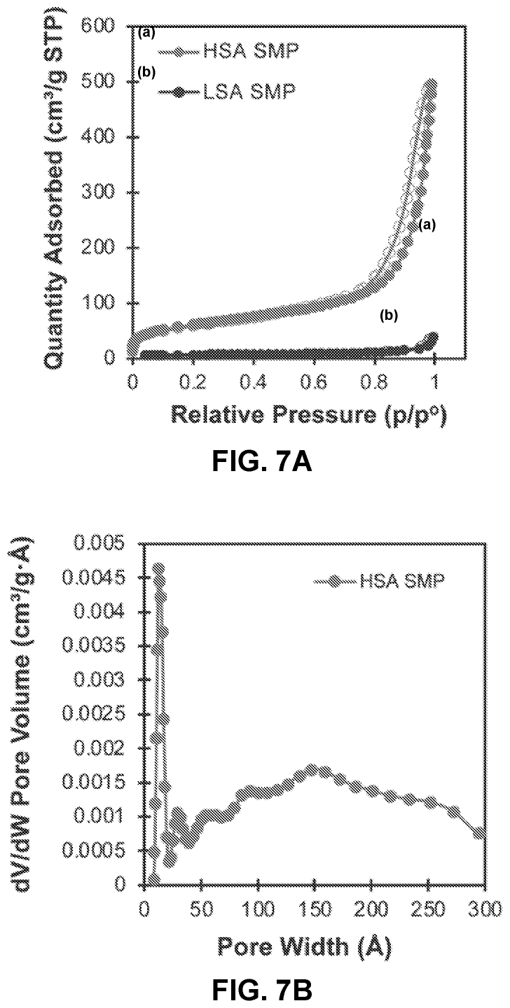

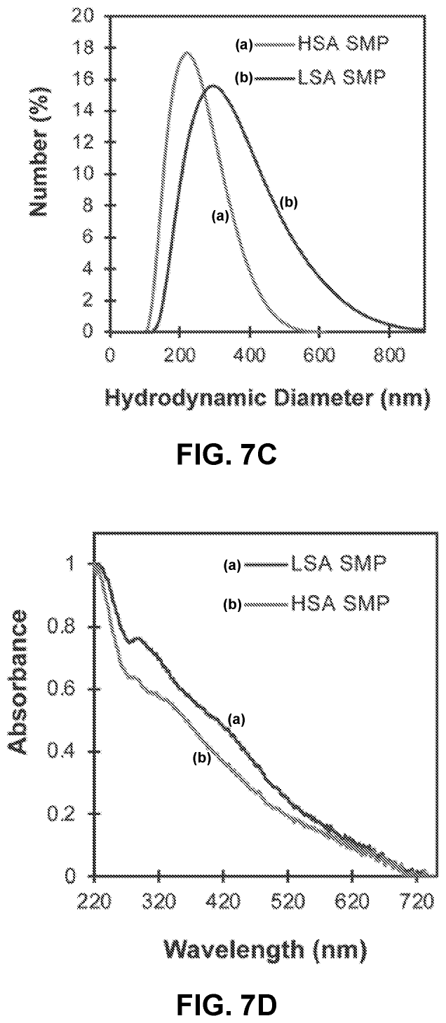

Porous and solid melanin mimics were synthesized through the oxidative polymerization of dopamine based on previously reported methods (FIGS. 1A-1G).²⁰ The surface area and morphology of the particles were characterized using N₂ sorption, dynamic light scattering, and ultraviolet-visible (UV-vis) spectroscopy (FIGS. 7A-7D). As previously reported, the High Surface Area Synthetic Melanin Particles (HSA-SMP) had a BET area of 190 m²/g with approximately 14 and 30 Å pores while the Low Surface Area Synthetic Melanin Particles (LSA-SMP) were non-porous with a BET area of 20 m²/g (FIGS. 7A-7B).²⁰ By DLS, HSA-SMP had a hydrodynamic diameter of 220±50 nm and LSA-SMP had a hydrodynamic diameter of 320±10 nm (FIG. 7C). Additionally, LSA and HSA-SMP looked the same by UV-vis spectroscopy (FIG. 7D). Both particles had an absorption maximum around 200 nm with a shoulder at 300 nm and a broad absorption tail. Other than porosity, the two particles had similar characteristics. Neither HSA or LSA-SMP could penetrate the stratum corneum of the skin (FIGS. 1E-1F).

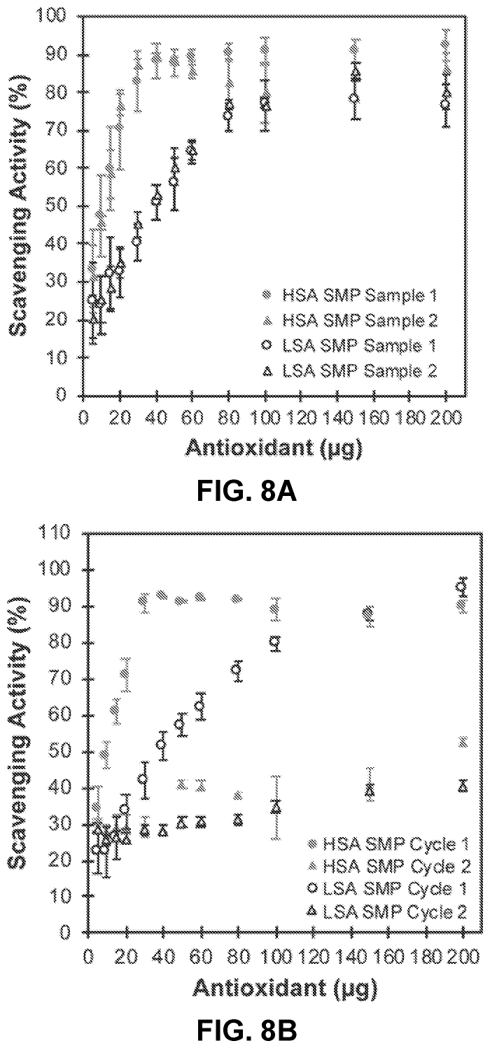

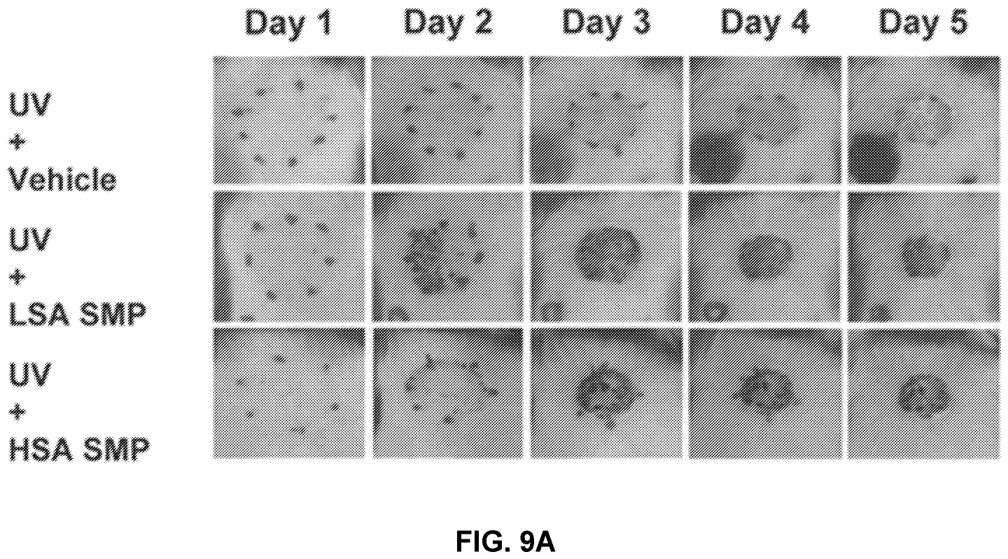

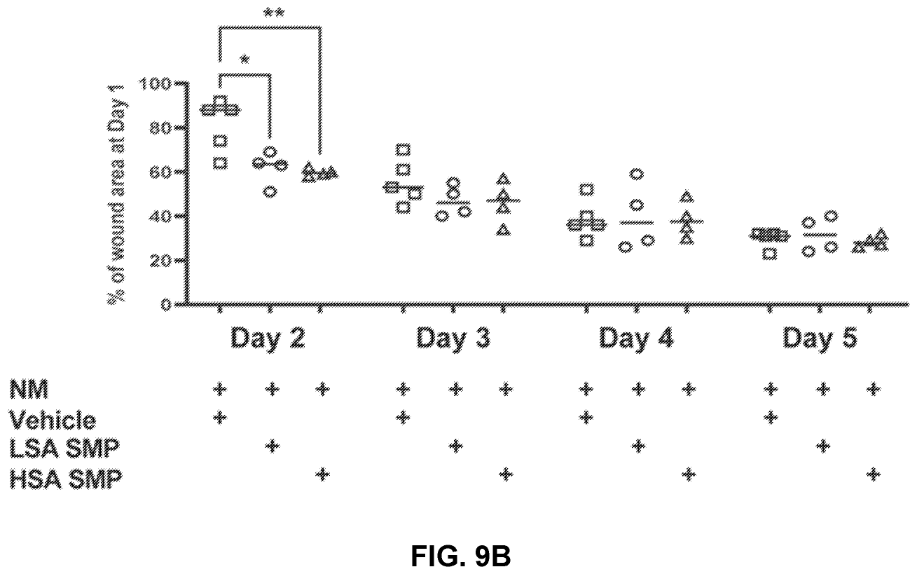

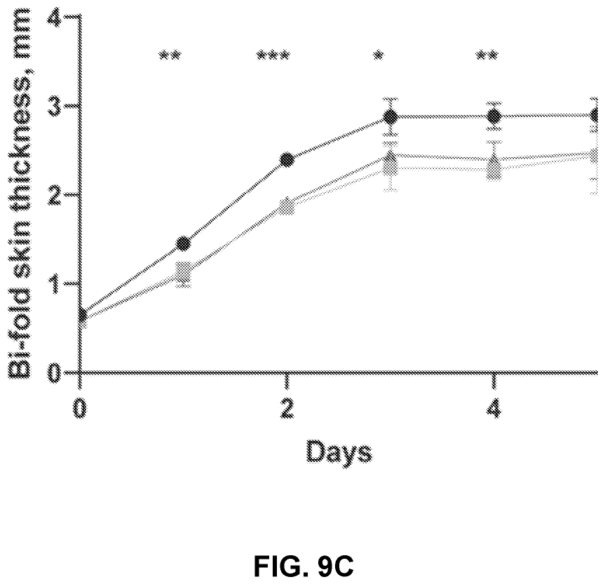

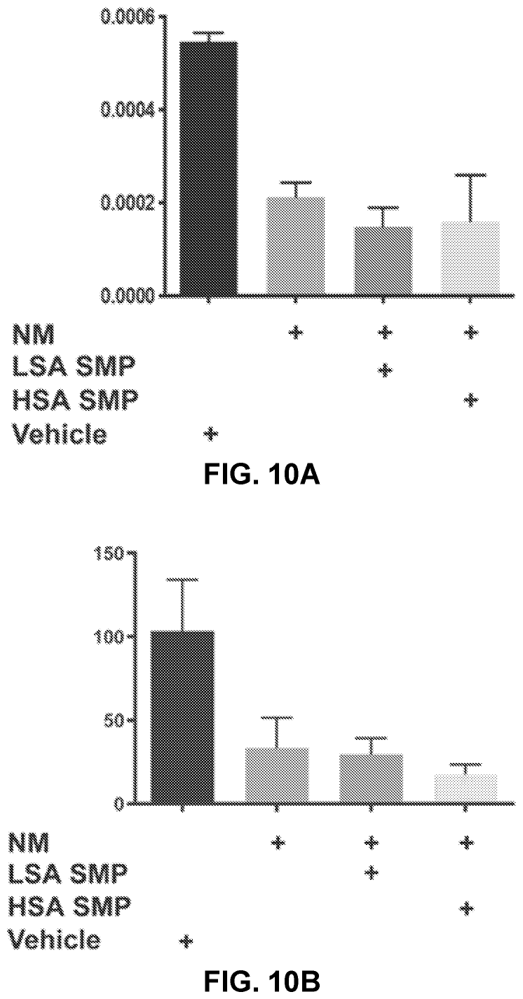

The scavenging activity of HSA-SMP and LSA-SMP was also assessed with a DPPH assay (FIG. 1G). HSA-SMP achieved a higher scavenging activity at lower particle concentrations than LSA-SMP. The scavenging activity of both particles plateaued around 100 ug, with HSA-SMP achieving approximately 90% scavenging activity and LSA-SMP achieving approximately 80%. Scavenging activity was consistent among different batches of particles (FIG. 8A). Scavenging activity decreased to about 40% for both SM particles when used a second time (FIG. 8B).

PDA Nanoparticle Treatment Improves Skin Healing after NM-Induced Injury:

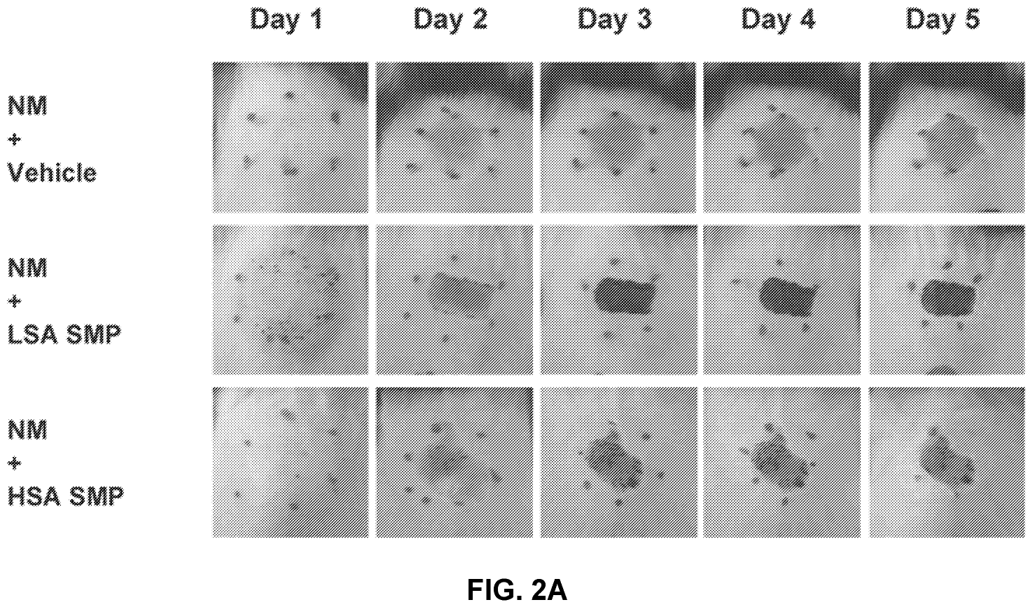

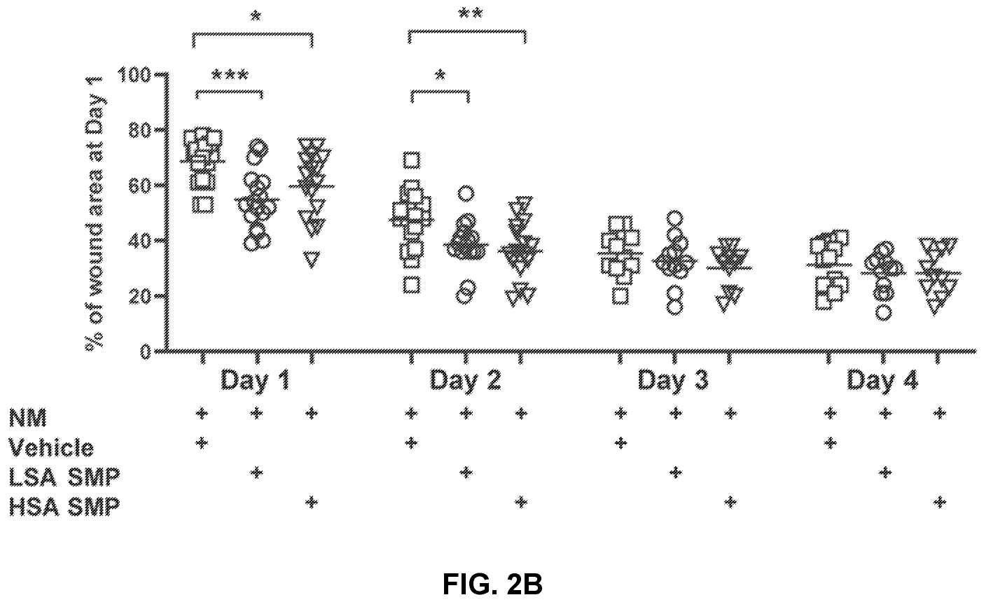

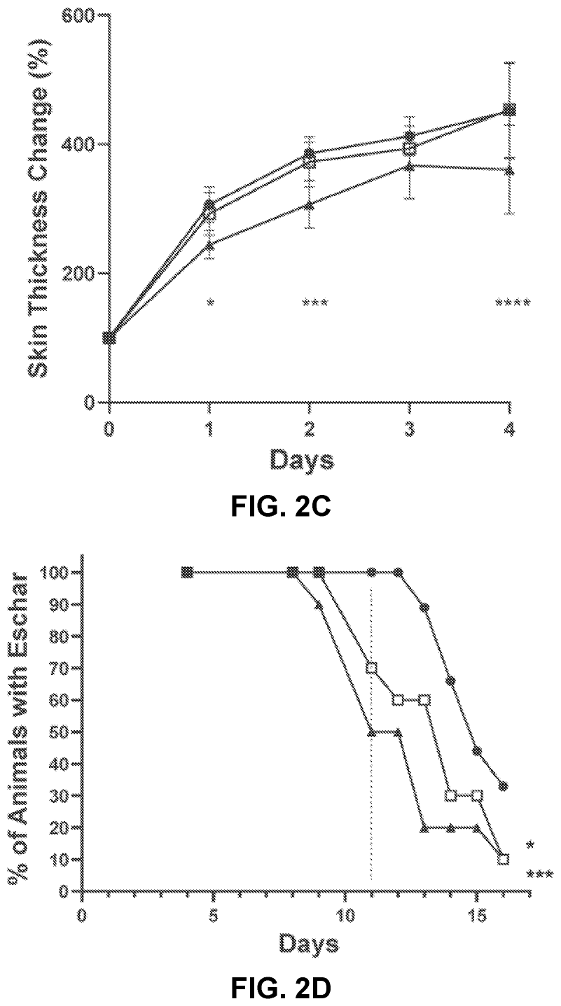

To test the hypothesis that treatment with topical SMP would improve wound healing, we utilized a NM-induced chemical injury mouse model. Topical NM was applied to shaved skin on each mouse. Two hours later, topical SMPs were applied to the wound site. Topical SMP application was repeated at 24 and 48 hours. The animals were monitored for up to 16 days. Bi-fold skin thickness, a proxy for skin edema, was measured until hard eschar formation prevented accurate measurements, usually a few days. Photos of the injury site were taken daily to track wound closure (FIG. 2A).

The treatment with either type of SMP improved wound healing, evidenced by the faster rates of wound area reduction (FIG. 2B) and time to eschar detachment (FIG. 2D) compared to the vehicle group. HSA-SMP-treated animals healed faster than both vehicle and LSA-SMP groups, with 50% of HSA mice demonstrating eschar detachment at 11 days post-injury compared to 30% and 0% in the LSA and vehicle groups, respectively (FIG. 2D). Furthermore, only HSA-SMP-treated animals experienced significantly reduced skin edema compared to vehicle-treated mice (FIG. 2C).

The same experiment was repeated using a UV radiation-induced injury model to confirm that physical adsorption of NM in SMP-treated mice had not influenced the severity of initial injury, despite our consideration for this effect by implementing a 2-hour temporal spacing. We found that again, SMP treatment resulted in reduced skin edema and faster wound closing rates.

PDA Nanoparticle Treatment Increases SOD Activity after NM Injury:

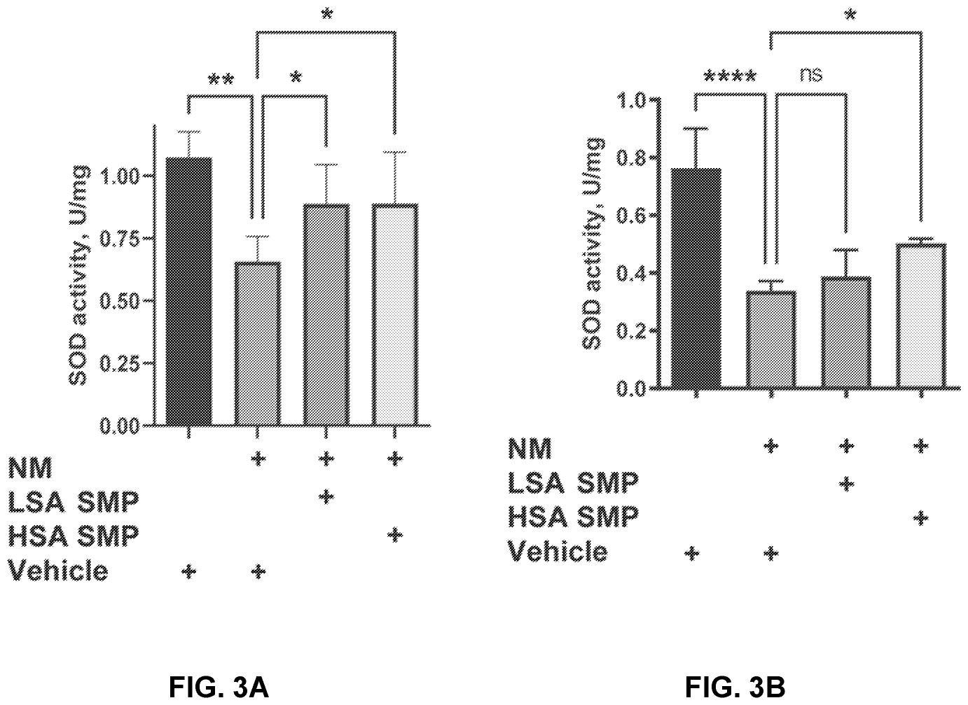

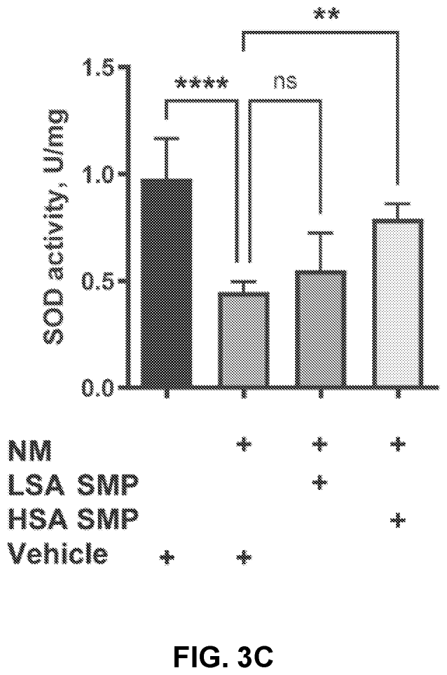

Skin injury leads to the excessive release of ROS. Antioxidative enzymes play a major role in the protection against the deleterious effects of ROS. One of the most important antioxidant enzymes is SOD. Since it is known that is affected by skin damage we evaluated how SMP treatment affected SOD activity. We assessed SOD activity from skin samples collected 24-, 48-, and 72-hours post-NM-injury using a commercially available colorimetric kit that measures the dismutation of superoxide radicals. We found that NM-treated mice had significantly lower SOD activity than control 24 hours post-injury (p<0.01). Treatment with either type of SMP significantly enhanced SOD activity compared to the vehicle group (p<0.05 for both) (FIG. 3A). At 48- and 72-hours post-injury, SMP-treated groups continued to demonstrate enhanced SOD activity over vehicle, although only HSA-SMP treatment achieved statistical significance.

To investigate whether other antioxidant enzymes were affected by SMP treatment, we measured the activity of thioredoxin reductase and catalase but did not find statistically significant differences between the experimental groups under certain conditions in embodiments.

### Porous PDA Nanoparticle Downregulate Inflammatory and Apoptosis Pathways:

Next, we used Taqman mouse immune arrays to identify possible mechanisms of SMP activity. Since each array card can only accommodate four samples, we pooled the genetic information from three mice per condition to create a single representative sample for each group. We repeated the assay three times, each using differently pooled samples from each group, except for the untreated group, which was used to bridge the obtained data for analysis. In the analysis, the NM+vehicle group was used as a reference.

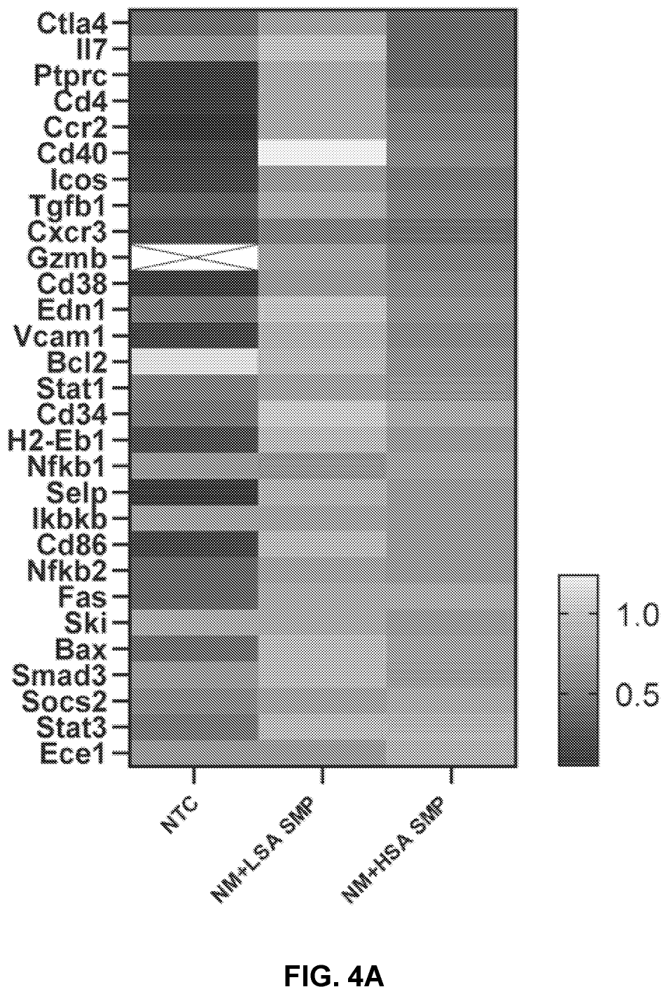

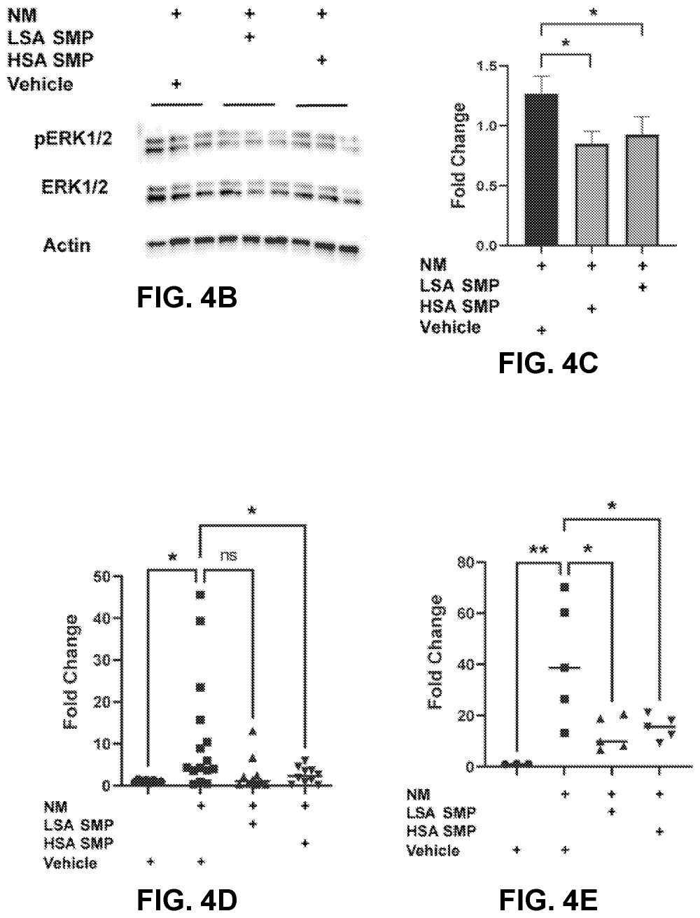

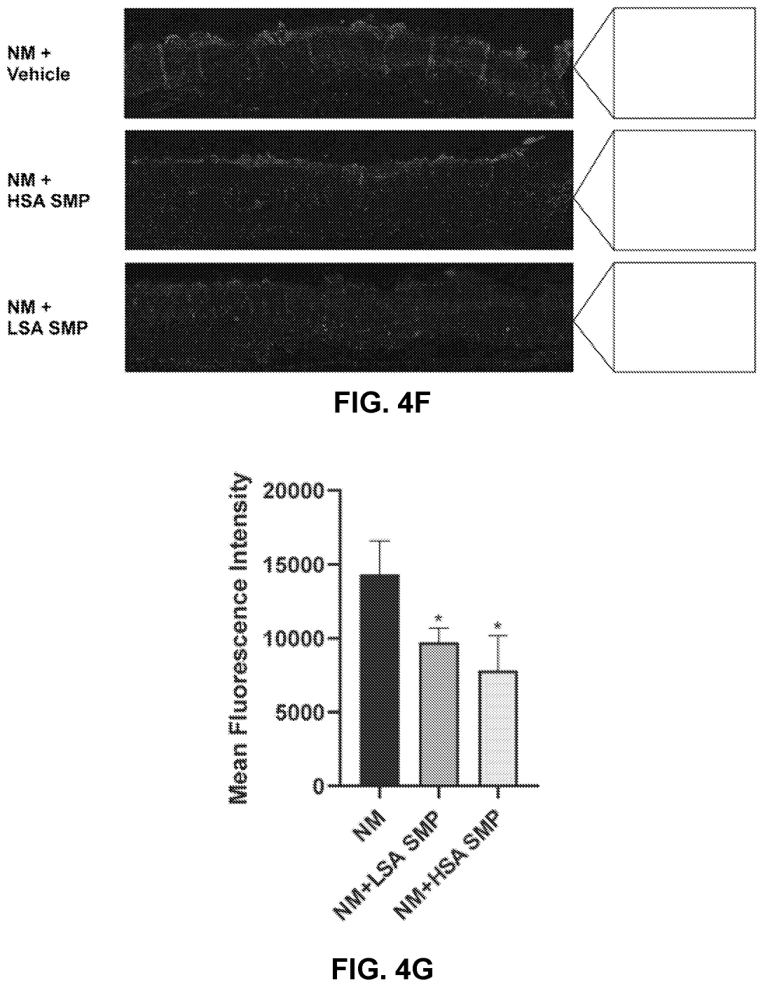

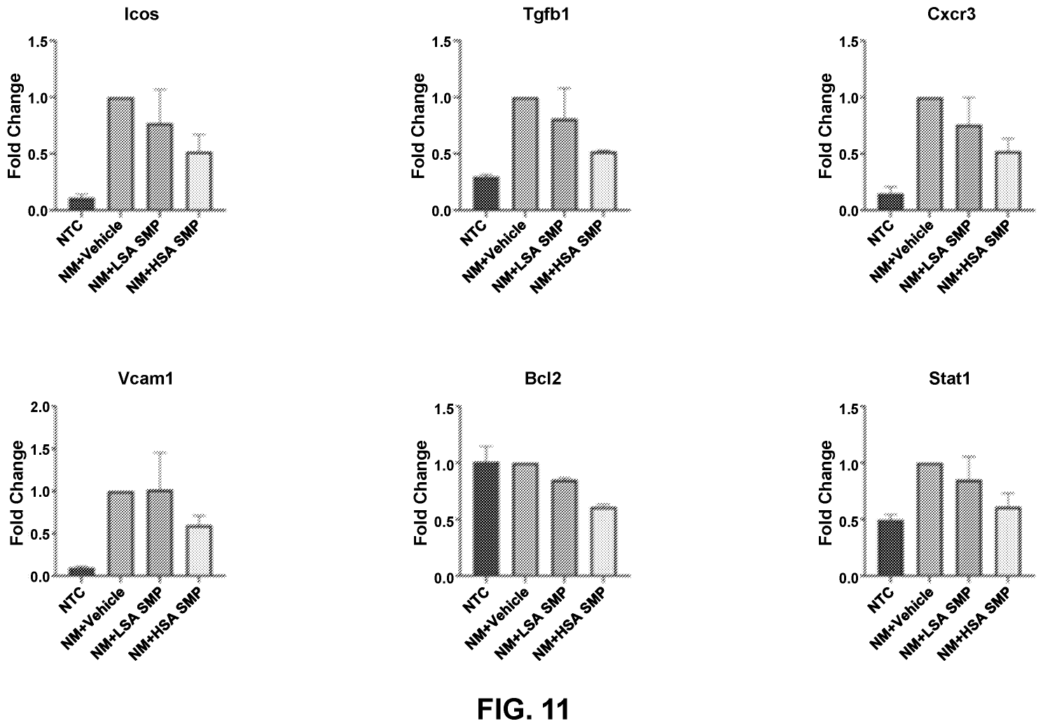

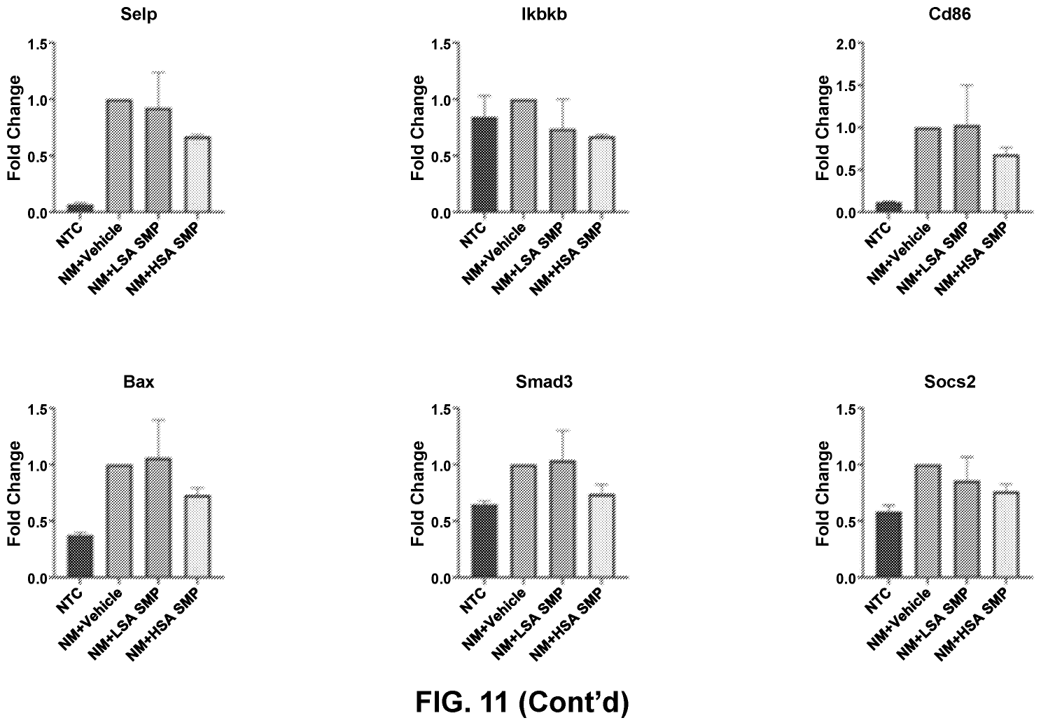

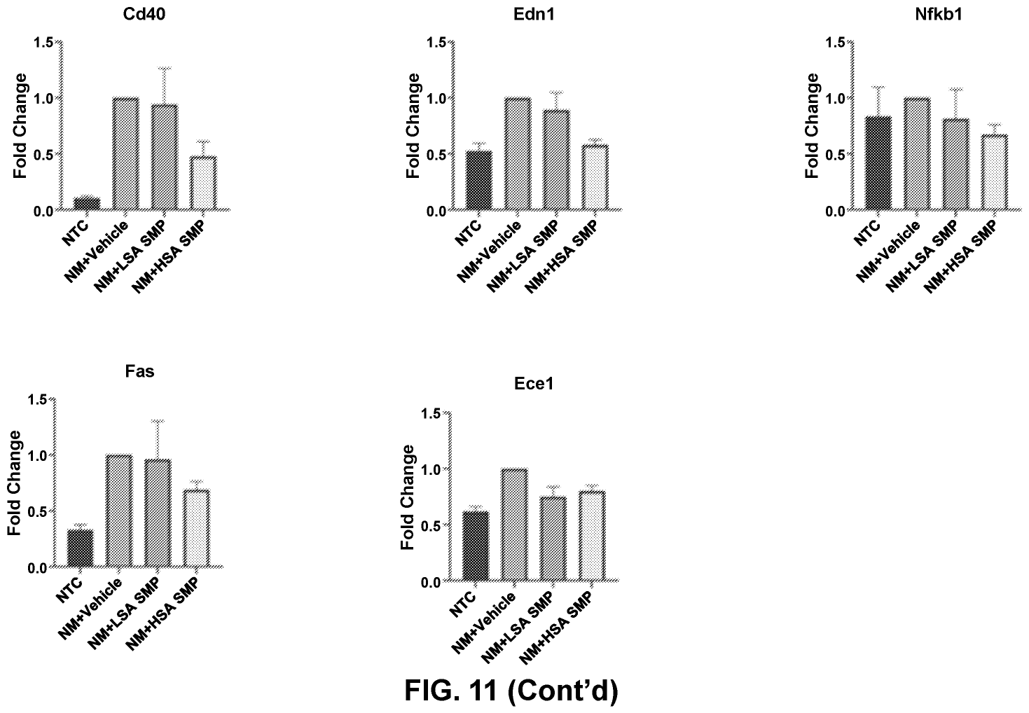

Following NM-induced injury, treatment with HSA SMP significantly downregulated 29 genes compared to the vehicle-treated animals (FIGS. 4A-4G and 11). In the LSA-SMP-treated group, only two genes (Bcl2 and Ece1) were downregulated with statistical significance due to the high variability between individual arrays, though the trend was toward downregulation of the inflammation-related genes (FIG. 11). Analysis of this gene expression data using the PANTHER Classification System identified “inflammation mediated by chemokine and cytokine signaling” and “apoptosis signaling” as two of the primary enriched pathways in SMP-treated mice, each including 7 differentially expressed genes compared to the vehicle group (FIG. 4A). See Table 1 for TaqMan Mouse Immune Array results corresponding to gene downregulation by HSA-SMP compared to the vehicle group corresponding to the classification “apoptosis signaling” and Table 2 for TaqMan Mouse Immune Array results corresponding to gene downregulation by HSA-SMP compared to the vehicle group corresponding to the classification “inflammation mediated by chemokine and cytokine signaling.”

TABLE 1
Genes, downregulated by HSA-SMP compared to vehicle group
identified as “Apoptosis signaling pathway”:

| | | Average ± st. dev. | % downregulation |
|---|---|---|---|
| Fas | NM + HSA-SMP | 0.69 ± 0.13 | (31%) |
| Gzmb | NM + HSA-SMP | 0.53 ± 0.03 | (47%) |
| Bcl2l1 | NM + HSA-SMP | 0.82 ± 0.11 | (18%) |
| Bcl2 | NM + HSA-SMP | 0.61 ± 0.04 | (39%) |
| Bax | NM + HSA-SMP | 0.73 ± 0.11 | (27%) |

TABLE 2
Genes, downregulated by HSA-SMP compared to vehicle
group identified as “Inflammation mediated by chemokine
and cytokine signaling pathway”:

| | | Average ± st. dev. | % downregulation |
|---|---|---|---|
| Cxcr3 | NM + HSA-SMP | 0.52 ± 0.19 | (48%) |
| Stat1 | NM + HSA-SMP | 0.61 ± 0.2 | (39%) |
| Stat3 | NM + HSA-SMP | 0.8 ± 0.12 | (20%) |
| Ccr2 | NM + HSA-SMP | 0.45 ± 0.12 | (55%) |

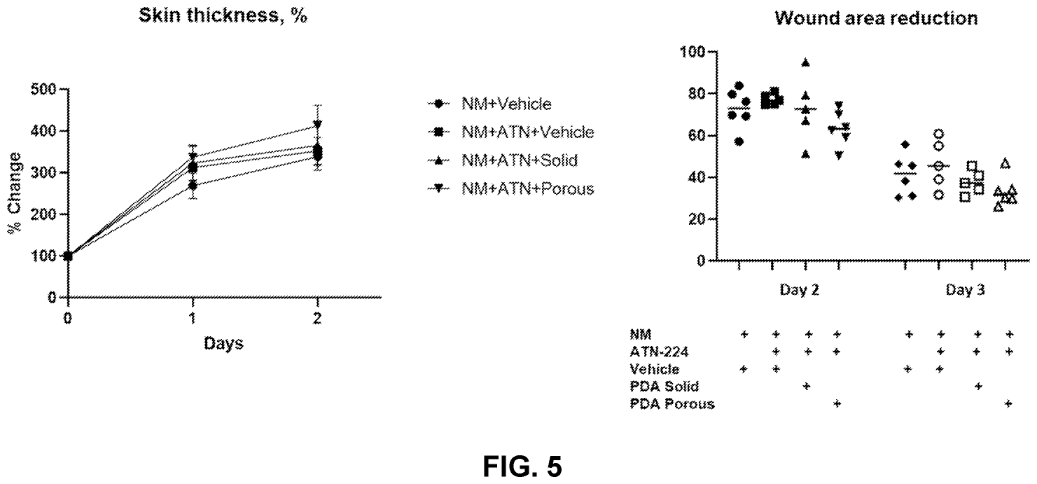

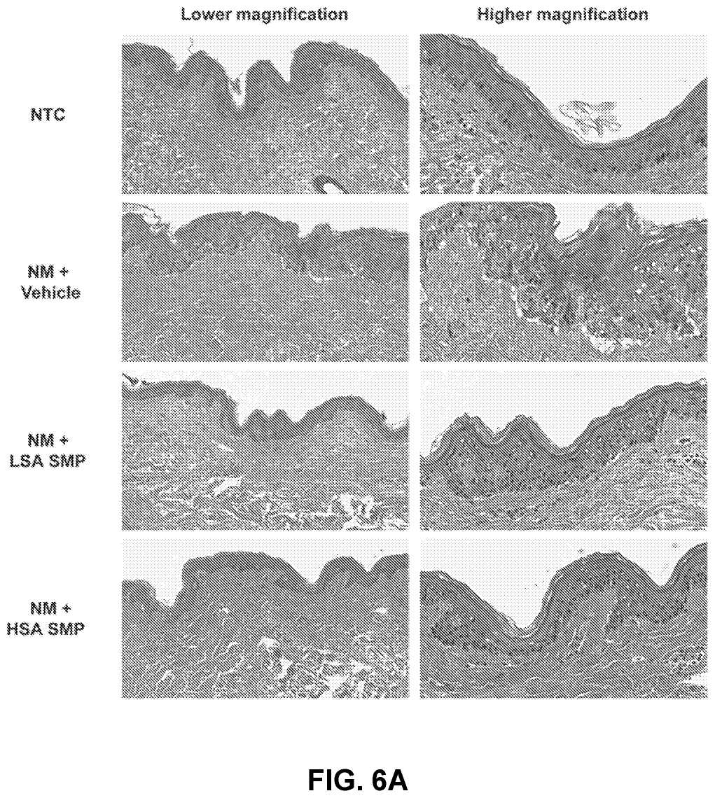

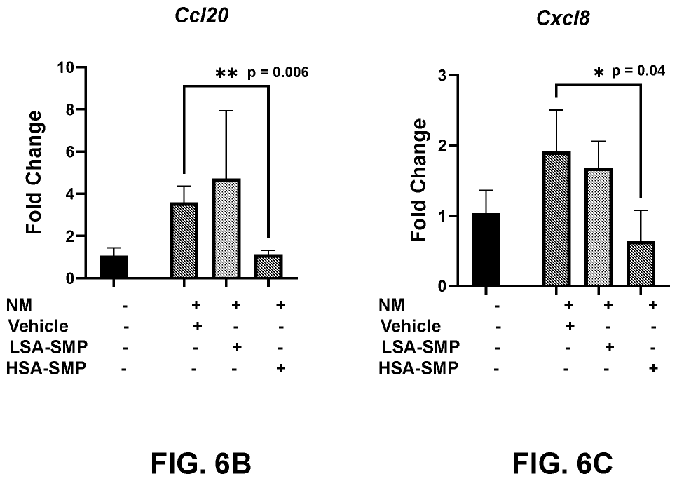

In addition to inducing oxidative stress, vesicating agents such as NM also induce pro-inflammatory signaling through the mitogen-activated protein kinase (MAPK) pathway [Kumar, 2015]. We performed western blotting analysis to determine the effect of SMP treatment on MAPK pathway (ERK1/2, p38, JNK) signaling. We found that treatment with either type of SMP significantly inhibited phosphorylation of ERK 1/2 (FIGS. 5A-5B) at 24 hours post-injury, while phosphorylation of p38 was unaffected. Since MAPK-pathway signaling regulates a myriad of inflammatory mediators at the level of translation and transcription [cite], we sought to determine if the SMP treatment-related signaling changes led to expression changes in known NM injury-associated proinflammatory mediators. The expression of MMP9 was significantly reduced by HSA-SMP at both 48 and 72 hours after injury and by LSA-SMP at 72 hour.

To confirm that SMP treatment inhibits apoptosis, we performed terminal deoxynucleotidyl transferase dUTP nick end labeling (TUNEL) staining of skin samples collected from each experimental group. As expected, NM injury alone resulted in strong TUNEL-positive staining (FIGS. 4A-4G). TUNEL staining was significantly reduced in mice treated with either SMP compared to the vehicle-only group by mean fluorescence intensity (p<0.05 for both), indicating decreased apoptosis (FIGS. 4A-4G).

### Inhibition of Cu/Zn SOD Partially Reverses SMP Effects on Skin Wound Healing

The sample preparation method we used for measuring SOD activity favors isolation of cytosolic SOD1, containing Cu and Zn. Therefore, to determine whether beneficial effects of SMP on wound repair were dependent on SOD1 activation, we repeated animal experiments in the presence of ATN-224, a small molecule copper-chelator and a well-known inhibitor of SOD1. Based on the literature data, lack of SOD1 is expected to have a negative effect on wound healing, it was contemplated to provide the treatment regimen (i.e., the dose of the inhibitor and the duration of treatment) that would produce relatively mild effect on wound healing, comparable to the animals not exposed to the inhibitor.

The images of the wounds are shown in FIG. 2A. The development of NM-induced edema was slightly more pronounced in all groups treated with ATN-224 compared to the PBS (vehicle for ATN-224) control group. We then confirmed that treatment with ATN-224 inhibited the upregulation of SOD activity in mouse skin. After confirming that treatment with ATN-224 inhibited the upregulation of SOD activity in our mice, we found that ATN-224 treatment also prevented any reduction in edema, dampened the improvement in wound-healing rate, and limited the inhibition of apoptosis previously observed in ATN-224−/SMP+ mice. However, both ATN-224+/SMP+ groups still healed faster than ATN-224+/SMP− groups, but with no statistically significant difference between SMP groups. Thus, the inhibition of the Cu/Zn SOD at least partially reversed the beneficial effects of PDA NP on skin wound healing.

### Synthetic Melanin Particle Treatment Alleviates NM-Induced Damage in Human Skin Explants:

We evaluated the effect of SMP treatment on injured human skin using ex vivo skin explants obtained from healthy donors undergoing abdominoplasty surgery. NM-induced injury resulted in characteristic histopathological features, including the disruption of epidermis-dermis connection, epidermal separation (vesciulation), and keratinocyte pyknosis.

It is also found that the exposure of healthy human skin to a therapeutic dose of NM (0.016% w/w mechlorethamine gel) resulted in the upregulation of a characteristic set of pro-inflammatory genes. The suppression of these genes by vitamin D3 treatment suggested their specific roles in the healing. It was contemplated whether the expression of this gene set was modified by SMP treatment in the human skin explants.

### Non-Limiting Discussion: Novel Particles, Topical Application:

Topical application of Synthetic Melanin Particles (SMP) improved skin healing after chemical and thermal injury. Melanin is a natural polymeric biomaterial that plays a role in skin protection against radiation. Due to its electron rich functional groups, melanin has complex redox capabilities allowing it to scavenge radical oxygen species and adsorb other harmful molecules that are generated in the skin during UV radiation exposure. This antioxidant activity of melanin is of particular interest as it has been previously shown to help with wound healing. Large accumulation of reactive oxygen species arising from wound damage can extend or prevent the transition from the inflammatory phase to the proliferative phase. Therefore, a biocompatible radical scavenging material is appealing toward tissue repair after chemical or thermal wounds.

Non-toxic biocompatible Synthetic Melanin Particles (SMP) were synthesized to mimic native melanin in the skin. Previous work was performed with melanin nanoparticles similar to the Low Surface Area particles (LSA-SMP) in 2D cell cultures of adult human keratinocyte cells (HEKa), which showed no toxicity or significant changes in cell viability.²¹ High Surface Area particles (HSA-SMP) with increased catechol concentrations and possible scavenging sites were synthesized to compare to Low Surface Area particles (LSA-SMP) and further elucidate melanin's role in wound healing. Although 2D keratinocyte cells can uptake SMP, neither LSA nor HSA-SMP penetrate the stratum corneum of the skin when applied topically, yet they still exhibit wound healing abilities including decreasing wound area size and skin thickness. Overall, both LSA and HSA-SMP exhibited wound healing effects with HSA-SMP outperforming LSA-SMP in certain cases. As seen in the radical scavenging DPPH assay, HSA-SMP has higher radical scavenging activity at lower concentrations and may be more efficient than LSA-SMP at quenching radicals and contributes to its superior performance.

### ROS; ROS in (Skin) Wounds:

ROS are highly reactive molecules in which 02 has been reduced with added electron The most studied molecules in this family include superoxide anion, peroxide, hydrogen peroxide, hydroxyl radicals and hydroxyl ions. During homeostasis, basal levels of ROS are tightly regulated and involved in maintain normal cell functioning, while change in ROS levels can induce pro-apoptotic signaling or cell-cycle arrest.

### Role of SOD, SOD1:

SOD family has three isoforms: Cu/Zn SOD1 is found mostly in the cytoplasm, the Mn/Zn SOD2 is in the mitochondrial matrix, and Cu/Zn SOD3 is located extracellularly.

### Inflammatory Pathways, Apoptosis:

Stimulation of MAPK pathways leads to activation of a number of various cellular events, including inflammatory responses, apoptosis, migration, proliferation, etc. It was shown that exposure of the mouse skin to vesicants such as sulfur mustard, NM and SEES results in the MAPK phosphorylation, supporting the idea that MAPKs play a role in the inflammatory response caused by these agents. Thus, according to Kumar et al., following NM injury in the mouse skin levels of pro-inflammatory molecules such as TNFα, iNOS, MMP9 were significantly increased, as well as phosphorylation of ERK1/2, p38 and JNK1/2. On the other hand, ROS was implicated in the activation of MAPK. The data demonstrates that decreasing the ROS burden by SMPs decreased pro-inflammatory signaling in the injured mouse skin, decreased ERK1/2 phosphorylation, and suppressed apoptosis.

### Example 1B: Exemplary, Non-Limiting, Materials and Methods Corresponds to Example 1A

Materials: Tetraethyl orthosilicate (TEOS) and 25 wt % poly(acrylic acid) solution (PAA) were purchased from Acros Organics. Hexadecyltrimethylammonium bromide (CTAB) was ordered from Tokyo Chemical Industry (TCI). Dopamine hydrochloride was obtained from Alfa Aesar. Ammonium hydroxide was purchased from Fisher Scientific. Hydrofluoric acid (HF), ethanol, and Trizma@Base (tris) were obtained from Sigma Aldrich. All materials were used as received without further purification.

Preparation of Mesoporous Silica Template: Mesoporous silica nanoparticles (MS) used as a template were synthesized based on a previous literature method.²² 0.55 g CTAB and 3.00 g PAA were dissolved under 25 mL of ultrapure water and stirred vigorously solution was clear. 2.0 g ammonium hydroxide was added to the stirring solution. After 20 min, 2.08 g TEOS was added and stirred for an additional 15 min. Then the solution was placed in an oven for 48 h at 120° C. The mixture was then centrifuged and dried before calcining the particles for 6 h at 550° C. to remove the remaining organic template.

Preparation of PDA Porous: PDA Porous was prepared based on a reported literature method.²⁰ 250 mg of MS was sonicated for 1 h in ultrapure water. To 225 mL of ultrapure water and 25 mL of ethanol (9:1 H₂O:EtOH by volume), 250 mg MS and 225 mg dopamine was added and stirred for 1 h at room temperature. Tris (10 mM, pH 8.5) was then added to the reaction and stirred for an additional 4 h. After the allotted time the reaction was centrifuged and washed with ultrapure water 5 times. The template was removed through a hydrofluoric acid (10 wt %) etch overnight, and then centrifuged and washed with ultrapure water 5 times.

Preparation of PDA Solid: PDA Solid was synthesized through the oxidative polymerization of dopamine. Briefly, 900 mg of dopamine was dissolved in 300 mL of ultrapure water and 4 mL of 1 M NaOH was added to the solution at room temperature and stirred for 18 h. The solution was then centrifuged and washed with ultrapure water 5 times.

DPPH Assay for Radical Scavenging Activity: DPPH radical scavenging activity of PDA Porous and Solid was determined according to a reported literature method.²³ 100 μL of PDA nanoparticles dispersed in water was added to an 1.8 mL solution of DPPH (0.2 mM in 95% ethanol). The total amount of PDA particles was varied from 5 to 200 μg. The solutions were left in the dark for 20 min. Afterwards the scavenging activity was monitored by taking the absorbance of the solutions at 516 nm. To determine the DPPH radical scavenging activity the following calculation (Eq. 1) was used.

$$I = \frac{1 - ( {A}_{i} - {A}_{j}}{{A}_{c}} \times 100 \% \qquad Eq . 1$$

I is the DPPH radical scavenging activity, A; is the absorbance of the samples with DPPH, Aⱼ is the absorbance of the samples without DPPH, and A_{c} is the absorbance of the DPPH without the PDA samples.

Characterization: All PDA particles were initially characterized by transmission electron microscopy (TEM, Hitachi HT-7700, 120 KV, or STEM, Hitachi HD-2300A, 200 KV). Dynamic light scattering (DLS, Malvern Instruments Ltd, Nano ZS), zeta-potential (Malvern Instruments Ltd, Nano ZS), and ultraviolet-visible spectroscopy (UV-Vis, Agilent Technologies Cary 100 UV-Vis) were used to investigate hydrodynamic diameters.

Sample Activation: Using a Micromeritics Smart VacPrep, samples were activated thermally under vacuum at 100° C. for the mesoporous silica and 75° C. for the pre-etched PDA Porous.

PDA samples were activated using a tousimis SAMDRI-PVT-3D Advanced Manual Critical Point Dryer. Prior to activation, samples were exchanged into ethanol overnight. Using the supercritical dryer, the sample was added to the sample chamber, cooled to 0-10° C., and pressurized to 800 psi. The ethanol was exchanged with liquid CO₂ over the course of 10 hours, purging the system for five minutes every two hours. After the fifth purge, the temperature was raised to 40° C. and the system was pressurized to 1200-1400 psi. The pressure was released slowly overnight at a rate of 0.5 cc/min. Samples were immediately transferred onto a Micromeritics Smart VacPrep and were placed under vacuum for two hours at 25° C. prior to sorption measurements.

Nitrogen Isotherms: For silica and pre-etched PDA Porous samples, N₂ isotherms were collected on a Micromeritics TriStar physisorption instrument at 77 K.

For PDA samples, nitrogen physisorption measurements were collected using a Micromeritics ASAP 2020 instrument at 77 K. Pore-size distributions were obtained using density functional theory (DFT) calculations with a carbon slit geometry and a N₂ DFT model.

Animals: All animal studies have been approved by the Northwestern University IACUC. Six to eight-week-old C57BL/6J female mice were purchased from Jackson Laboratories.

Nitrogen Mustard Skin Injury Model: Dorsal area of the mice was shaved and chemically depilated 48 hours before skin injury induction. Mice were anesthetized and placed on a heat pad under a chemical fume hood. 0.5% of mechloroethamine hydrochloride (nitrogen mustard, NM) (Sigma, 122564) solution in 1.5% DMSO-PBS was prepared immediately before the application. Total of 40 μl of NM solution was applied on a circular (12 mm diameter) area in two consecutive applications. After the application mice were placed in a temporary housing space under a chemical fume hood for two hours.

UV Radiation Skin Injury Model: Mice were exposed to UV radiation as described previously [X]. Briefly, a 12 mm diameter circular area of back skin depilated of hair was exposed to UVB irradiation from six FS-40 fluorescent lamps filtered through Kodacel (Eastman Kodak Co., Rochester, NY). UVB emission was measured with an IL-443 phototherapy radiometer (International Light, Newburyport, MA) furnished with an IL SED 240 detector. Mice were exposed to a single UVB dose of 100 mJ/cm2 to induce skin inflammation.

Treatment with Polydopamine Nanoparticles (PDA NPs): PDA NPs were diluted in milli-Q water at concentration of 50 μg/μl. Total amount of 1 mg of particles were applied to the injured skin area two hours after the injury induction, and then 24 and 48 hours later. Milli-Q water was used as a vehicle in control groups. During the treatment mice were anesthetized using isoflurane.

Monitoring Skin Injury and Measurement of Wound Healing: Mice were followed up non-invasively after induction of skin injury. Monitoring was performed daily, starting at the day of skin injury. Photographs of the injured area were taken, the bi-fold skin thickness of the injured area was measured using a digital calipers (Mitutoyo, PK0505CPX), and weight was measured. The area of the inflammation/wound was measured using Image J and QuPath software.

Statistical Analysis: GraphPad Prism V.8.3.0 software was used to create visual graphics and to calculate the statistical significance. One-way ANOVA and t-test were used to calculate the p-values.

References corresponding to Example 1A and Example 1B:
1. D'Alba, L.; Shawkey, M. D., Melanosomes: Biogenesis, Properties, and Evolution of an Ancient Organelle. Physiol. Rev. 2019, 99 (1), 1-19.
2. Simon, J. D.; Peels, D. N., The Red and the Black. Acc. Chem. Res. 2010, 43 (11), 1452-1460.
3. Solano, F., Melanins: Skin Pigments and Much More-Types, Structural Models, Biological Functions, and Formation Routes. New Sci. J. 2014, 2014, 1-28.
4. Jakubiak, P.; Lack, F.; Thun, J.; Urtti, A.; Alvarez-Sanchez, R., Influence of Melanin Characteristics on Drug Binding Properties. Mol Pharm 2019, 16 (6), 2549-2556.
5. Razanowska, M.; Sarna, T.; Land, E. J.; Truscott, T. G., Free radical scavenging properties of melanin: Interaction of eu- and pheo-melanin models with reducing and oxidising radicals. Free Radic Biol Med 1999, 26 (6), 518-525.
6. Kim, K.; Tsay, O. G.; Atwood, D. A.; Churchill, D. G., Destruction and Detection of Chemical Warfare Agents. Chem. Rev. 2011, 111 (9), 5345-403.
7. Smart, J. K., Medical Aspects of Chemical and Biological Warfare. Office of the Surgeon General: Washington, DC, 1997.
8. Tu, A. T., Natural and Selected Synthetic Toxins. Tu, A. T.; Gaffield, W., Eds. American Chemical Society: Washington DC, 2000; Vol. 745.
9. Makarovsky, I.; Markel, G.; Hoffman, A.; Schein, O.; Finkelstien, A.; Brosh-Nissimov, T.; Tashma, Z.; Dushnitsky, T.; Eisenkraft, A., Osmium Tetroxide: A New Kind of Weapon. Isr. Med. Assoc. J. 2007, 9 (10), 750-752.
10. Saladi, R. N.; Smith, E.; Persaud, A. N., Mustard: a potential agent of chemical warfare and terrorism. Clin Exp Dermatol 2006, 31 (1), 1-5.
11. Ryu, J. H.; Messersmith, P. B.; Lee, H., Polydopamine Surface Chemistry: A Decade of Discovery. ACS Appl. Mater. Interfaces 2018, 10 (9), 7523-7540.
12. Siwicka, Z. E.; Son, F. A.; Battistella, C.; Moore, M. H.; Korpanty, J.; McCallum, N. C.; Wang, Z.; Johnson, B. J.; Farha, O. K.; Gianneschi, N. C., Synthetic Porous Melanin. Journal of the American Chemical Society 2021, 143 (8), 3094-3103.
13. Huang, L.; Liu, M.; Huang, H.; Wen, Y.; Zhang, X.; Wei, Y., Recent Advances and Progress on Melanin-like Materials and Their Biomedical Applications. Biomacromolecules 2018, 19 (6), 1858-1868.
14. Wang, Z.; Carniato, F.; Xie, Y.; Huang, Y.; Li, Y.; He, S.; Zang, N.; Rinehart, J. D.; Botta, M.; Gianneschi, N. C., High Relaxivity Gadolinium-Polydopamine Nanoparticles. Small 2017, 13 (43).
15. Zhang, R.; Fan, Q.; Yang, M.; Cheng, K.; Lu, X.; Zhang, L.; Huang, W.; Cheng, Z., Engineering Melanin Nanoparticles as an Efficient Drug-Delivery System for Imaging-Guided Chemotherapy. Advanced Materials 2015, 27 (34), 5063-5069.
16. Cheng, W.; Nie, J.; Gao, N.; Liu, G.; Tao, W.; Xiao, X.; Jiang, L.; Liu, Z.; Zeng, X.; Mei, L., A Multifunctional Nanoplatform against Multidrug Resistant Cancer: Merging the Best of Targeted Chemo/Gene/Photothermal Therapy. Advanced Functional Materials 2017, 27 (45), 1704135.
17. Wang, J.-G.; Zhou, H.-J.; Sun, P.-C.; Ding, D.-T.; Chen, T.-H., Hollow Carved Single-Crystal Mesoporous Silica Templated by Mesomorphous Polyelectrolyte-Surfactant Complexes. Chem. Mater. 2010, 22 (13), 3829-3831.
18. Ju, K.-Y.; Lee, Y.; Lee, S.; Park, S. B.; Lee, J.-K., Bioinspired Polymerization of Dopamine to Generate Melanin-Like Nanoparticles Having an Excellent Free-Radical-Scavenging Property. Biomacromolecules 2011, 12 (3), 625-632.

### ADDITIONAL REFERENCES

1. D'Alba, L.; Shawkey, M. D., Melanosomes: Biogenesis, Properties, and Evolution of an Ancient Organelle. Physiol. Rev. 2019, 99 (1), 1-19.
2. Van Den Bossche, K.; Naeyaert, J.-M.; Lambert, J., The Quest for the Mechanism of Melanin Transfer. Traffic 2006, 7 (7), 769-778.
3. Simon, J. D.; Peels, D. N., The Red and the Black. Acc. Chem. Res. 2010, 43 (11), 1452-1460.
4. Solano, F., Melanins: Skin Pigments and Much More—Types, Structural Models, Biological Functions, and Formation Routes. New Sci. J. 2014, 2014, 1-28.
5. D'Alba, L.; Van Hemert, C.; Spencer, K. A.; Heidinger, B. J.; Gill, L.; Evans, N. P.; Monaghan, P.; Handel, C. M.; Shawkey, M. D., Melanin-based color of plumage: role of condition and of feathers' microstructure. Integr Comp Biol 2014, 54 (4), 633-44.
6. Goiran, C.; Bustamante, P.; Shine, R., Industrial Melanism in the Seasnake Emydocephalus Annulatus. Curr. Biol. 2017, 27 (16), 2510-2513.
7. Schroeder, R. L.; Gerber, J. P., Chloroquine and Hydroxychloroquine Binding to Melanin: Some Possible Consequences for Pathologies. Toxicol. Rep. 2014, 1, 963-968.
8. Jakubiak, P.; Lack, F.; Thun, J.; Urtti, A.; Alvarez-Sanchez, R., Influence of Melanin Characteristics on Drug Binding Properties. Mol Pharm 2019, 16 (6), 2549-2556.
9. Price, R. J.; Lee, J. S., Paralytic Shellfish Poison and Melanin Distribution in Fractions of Toxic Butter Clam (Saxidomus giganteus) Siphon. Can. J. Fish. Aquat. Sci. 1972, 29 (11), 1657-1658.
10. Margalida, A.; Negro, J. J.; Galvan, I., Melanin-based color variation in the Bearded Vulture suggests a thermoregulatory function. Comp Biochem Physiol A Mol Integr Physiol 2008, 149 (1), 87-91.
11. Razanowska, M.; Sarna, T.; Land, E. J.; Truscott, T. G., Free radical scavenging properties of melanin: Interaction of eu- and pheo-melanin models with reducing and oxidising radicals. Free Radic Biol Med 1999, 26 (6), 518-525.
12. Burgoyne, T.; O'Connor, M. N.; Seabra, M. C.; Cutler, D. F.; Futter, C. E., Regulation of melanosome number, shape and movement in the zebrafish retinal pigment epithelium by OA1 and PMEL. Journal of Cell Science 2015, 128 (7), 1400-1407.
13. Puertas-Bartolomé, M.; Benito-Garzón, L.; Fung, S.; Kohn, J.; Vezquez-Lasa, B.; San Romen, J., Bioadhesive functional hydrogels: Controlled release of catechol species with antioxidant and antiinflammatory behavior. Materials Science and Engineering: C 2019, 105, 110040.
14. Bagheri, M.; Validi, M.; Gholipour, A.; Makvandi, P.; Sharifi, E., Chitosan nanofiber biocomposites for potential wound healing applications: Antioxidant activity with synergic antibacterial effect. Bioengineering & Translational Medicine 2022, 7 (1).
15. Wang, Z.; Carniato, F.; Xie, Y.; Huang, Y.; Li, Y.; He, S.; Zang, N.; Rinehart, J. D.; Botta, M.; Gianneschi, N. C., High Relaxivity Gadolinium-Polydopamine Nanoparticles. Small 2017, 13 (43).
16. Zhang, R.; Fan, Q.; Yang, M.; Cheng, K.; Lu, X.; Zhang, L.; Huang, W.; Cheng, Z., Engineering Melanin Nanoparticles as an Efficient Drug-Delivery System for Imaging-Guided Chemotherapy. Advanced Materials 2015, 27 (34), 5063-5069.
17. Cheng, W.; Nie, J.; Gao, N.; Liu, G.; Tao, W.; Xiao, X.; Jiang, L.; Liu, Z.; Zeng, X.; Mei, L., A Multifunctional Nanoplatform against Multidrug Resistant Cancer: Merging the Best of Targeted Chemo/Gene/Photothermal Therapy. Advanced Functional Materials 2017, 27 (45), 1704135.
18. Bryan, N.; Ahswin, H.; Smart, N.; Bayon, Y.; Wohlert, S.; Hunt, J., Reactive oxygen species (ROS)—a family of fate deciding molecules pivotal in constructive inflammation and wound healing. European Cells and Materials 2012, 24, 249-265.
19. Dunnill, C.; Patton, T.; Brennan, J.; Barrett, J.; Dryden, M.; Cooke, J.; Leaper, D.; Georgopoulos, N. T., Reactive oxygen species (ROS) and wound healing: the functional role of ROS and emerging ROS-modulating technologies for augmentation of the healing process. International Wound Journal 2017, 14 (1), 89-96.
20. Siwicka, Z. E.; Son, F. A.; Battistella, C.; Moore, M. H.; Korpanty, J.; McCallum, N. C.; Wang, Z.; Johnson, B. J.; Farha, O. K.; Gianneschi, N. C., Synthetic Porous Melanin. Journal of the American Chemical Society 2021, 143 (8), 3094-3103.
21. Huang, Y.; Li, Y.; Hu, Z.; Yue, X.; Proetto, M. T.; Jones, Y.; Gianneschi, N. C., Mimicking Melanosomes: Polydopamine Nanoparticles as Artificial Microparasols. ACS Cent. Sci. 2017, 3 (6), 564-569.
22. Wang, J.-G.; Zhou, H.-J.; Sun, P.-C.; Ding, D.-T.; Chen, T.-H., Hollow Carved Single-Crystal Mesoporous Silica Templated by Mesomorphous Polyelectrolyte-Surfactant Complexes. Chem. Mater. 2010, 22 (13), 3829-3831.
23. Ju, K.-Y.; Lee, Y.; Lee, S.; Park, S. B.; Lee, J.-K., Bioinspired Polymerization of Dopamine to Generate Melanin-Like Nanoparticles Having an Excellent Free-Radical-Scavenging Property. Biomacromolecules 2011, 12 (3), 625-632.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Certain molecules disclosed herein may contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every material, formulation, combination of materials, and method described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

We claim:

1. A method for treatment of a subject, the method comprising:
topically administering a melanin formulation having an artificial melanin material to damaged skin of the subject;
wherein the administered artificial melanin material comprises an extracellular artificial melanin material at the damaged skin; and
facilitating skin healing at the damaged skin via at least the extracellular artificial melanin material;
wherein the step of facilitating skin healing comprises at least a portion of the extracellular artificial melanin material performing a therapeutic extracellular activity.

2. The method of claim 1, wherein the damaged skin comprises a closed wound, thermally-induced damage, chemically-induced damage, radiation-induced damage, mechanical-friction damage, and/or infection cellulitis-induced damage, one or more blisters, or any combination thereof.

3.-4. (canceled)

5. The method of claim 1, wherein at least a portion of the extracellular melanin material is in the stratum corneum of the damaged skin.

6. The method of claim 1, wherein the damaged skin comprises extracellular free radical species; and wherein the therapeutic extracellular activity comprises the at least a portion of the extracellular artificial melanin material quenching at least the extracellular free radical species.

7. The method of claim 6, wherein the extracellular free radical species comprise reactive oxygenated species.

8. (canceled)

9. The method of claim 1, wherein the damaged skin comprises inflammation; and wherein the step of facilitating skin healing comprises at least a portion of the administered artificial melanin material directly and/or indirectly reducing the inflammation.

10. (canceled)

11. The method of claim 9, wherein directly and/or indirectly reducing the inflammation comprises at least a portion of the administered artificial melanin adsorbing one or more inflammatory factors and/or one or more enzymatic factors.

12. (canceled)

13. The method of claim 11, wherein the one or more inflammatory factors and/or one or more enzymatic factors comprise TNFα, iNOS, MMP9, one or more proteins associated with the MAPK/ERK pathway, and/or one or more enzymes associated with the MAPK/ERK pathway.

14. (canceled)

15. The method of claim 1, wherein the step of facilitating comprises at least a portion of the administered artificial melanin directly and/or indirectly downregulating inflammation-related genes and/or apoptosis-related genes compared to when the artificial melanin materials is absent.

16. (canceled)

17. The method of claim 1, wherein the step of facilitating skin healing further comprises at least a portion of the administered artificial melanin material performing a therapeutic intracellular activity.

18. The method of claim 17, wherein the therapeutic intracellular activity comprises:
quenching intracellular free radical species; and/or
adsorbing one or more intracellular inflammatory factors and/or one or more intracellular enzymatic factors.

19.-21. (canceled)

22. The method of claim 1, wherein the artificial melanin material comprises porous artificial melanin material, artificial melanin particles, porous artificial melanin particles, or any combination thereof.

23.-24. (canceled)

25. The method of claim 1, wherein at least a portion of the artificial melanin material comprises polydopamine, eumelanin, pheomelanin, selenomelanin, allomelanin, or a combination of these.

26. (canceled)

27. The method of claim 1, wherein the artificial melanin material comprises a plurality of melanin oligomers and/or polymers; and wherein each melanin oligomer and/or polymer comprises a plurality of covalently-bonded melanin base units.

28. The method of claim 27, wherein said melanin base units are one or more substituted or unsubstituted catechol-based monomer units, substituted or unsubstituted polyol-based monomer units, substituted or unsubstituted phenol-based monomer units, substituted or unsubstituted indole-based monomer units, substituted or unsubstituted benzothiazine-based monomer units, substituted or unsubstituted benzothiazole-based monomer units, substituted or unsubstituted dopamine-based monomer units, or any combination of these.

29.-44. (canceled)

45. The method of claim 1, wherein the artificial melanin material comprises artificial melanin particles having a size selected from the range of 10 nm to 1000 nm.

46.-48. (canceled)

49. The method of claim 1, wherein the melanin formulation is hydrophilic and/or wherein the artificial melanin material is hydrophilic.

50.-52. (canceled)

53. The method of claim 1, wherein the melanin formulation is free of artificial melanin material loaded or functionalized with a non-melanin therapeutic agent and/or hollow and/or semi-hollow melanin particles carrying a non-melanin therapeutic agent.

54.-59. (canceled)

60. The method of claim 1, wherein at least 50% of the administered artificial melanin material is the extracellular artificial melanin material.

61. The method of claim 1, wherein the extracellular artificial melanin material is present extracellularly as long as being present at the damaged skin.
